(19) United States
(12) Patent Application Publication
(10) Pub. No.: US 2016/0017347 A1
(43) Pub. Date: Jan. 21, 2016

(54) TERMINATING FLOWER (TMF) GENE AND METHODS OF USE

(71) Applicants: E. I. DU PONT DE NEMOURS AND COMPANY, Wilmington, DE (US); COLD SPRING HARBOR LABORATORY, Cold Spring Harbor, NY (US)

(72) Inventors: Stephen M. Allen, Wilmington, DE (US); Zachary B. Lippman, North Bellmore, NY (US); Cora A. Macalister, Huntington Station, NY (US)

(21) Appl. No.: 14/409,990

(22) PCT Filed: Jun. 17, 2013
(86) PCT No.: PCT/US2013/046098

§ 371 Date: Dec. 19, 2014

Related U.S. Application Data
(60) Provisional application No. 61/662,023, filed on Jun. 20, 2012.

(51) Int. Cl. C12N 15/82; C07K 14/415

(52) U.S. Cl. CPC C12N 15/827; C12N 15/8261; C12N 15/8218; C07K 14/415

(57) ABSTRACT

Described herein are the following: isolated polynucleotides, isolated polypeptides and recombinant DNA constructs; compositions (such as plants or seeds) comprising these recombinant DNA constructs; and methods of use for these recombinant DNA constructs. The recombinant DNA construct comprises a polynucleotide operably linked to a promoter that is functional in a plant, wherein said polynucleotide encodes a TMF polypeptide.

This application claims the benefit of U.S. Provisional Application No. 61/662,023, filed Jun. 20, 2012, the entire content of which is herein incorporated by reference.

### FIELD OF THE INVENTION

The field of invention relates to plant biotechnology, plant breeding and genetics and, in particular, relates to recombinant DNA constructs useful in plants for altering floral development.

### BACKGROUND OF THE INVENTION

Variation in plant reproductive success and agricultural productivity is largely determined by differences in shoot architecture, and reproductive shoots known as inflorescences show extensive diversity for both branch and flower number. Inflorescences arise from vegetative shoots when endogenous and environmental signals coincide to induce pluripotent cells at growing tips called shoot apical meristems (SAM) to transition to flower-producing inflorescence meristems (IM) (Kobayashi, Y. and D. Weigel, Move on up, it's time for change—mobile signals controlling photoperiod-dependent flowering. Genes Dev, 2007. 21(19): p. 2371-84; Turck, F., F. Fornara, and G. Coupland, Regulation and identity of florigen: FLOWERING LOCUS T moves center stage. Annu Rev Plant Biol, 2008. 59: p. 573-94). Flowering transitions and their impacts on subsequent meristem growth and shoot architecture can vary greatly, and one of the most dramatic, yet poorly understood, differences in meristem growth and resulting shoot organization involves ‘monopodial’ versus ‘sympodial’ growth programs. In monopodial plants such as Arabidopsis and maize, the SAM persists after the transition to flowering, and the IM continuously generates lateral flowers, resulting in a limited range of simple shoot architectures. In contrast, in sympodial plants such as tomato and related nightshades (Solanaceae), the primary meristem ends growth by terminating in a flower, and new growth arises from specialized axillary meristems called sympodial meristems that also undergo floral termination to produce compound shoots (Knaap, E., et al., Solanaceae—a model for linking genomics with biodiversity. Comp. Funct. Genom., 2004. 5: p. 285-291; Pnueli, L., et al., The SELF-PRUNING gene of tomato regulates vegetative to reproductive switching of sympodial meristems and is the ortholog of CEN and TFL1. Development, 1998. 125(11): p. 1979-89; Lifschitz, E. and Y. Eshed, Universal florigenic signals triggered by FT homologues regulate growth and flowering cycles in perennial day—neutral tomato. J Exp Bot, 2006. 57(13): p. 3405-14). While domesticated tomatoes (Solanum lycopersicum) generate compound inflorescences with several flowers arranged in a zigzag architecture, inflorescences of wild tomato species like S. lycopersicoides produce multiple branches with dozens of flowers. At the other extreme are Solanaceae species like pepper (Capsicum annuum), petunia (Petunia hybrida), and the tobacco relative Nicotiana benthamiana, whose inflorescences are composed of solitary flowers. The basis for this remarkable range of inflorescence complexity remains poorly understood, but it has previously been shown that mutations in the homeobox transcription factor gene COMPOUND INFLORESCENCE (S/WOX9) and the floral specification complex encoded by the F-box gene ANANTHA/UNUSUAL FLORAL ORGANS (AN/UFO) and its transcription factor partner FALSIFLORA/LEAFY (FA/LFY) cause highly branched inflorescences by delaying (s mutants) or blocking (an and fa mutants) floral termination (Lippman, Z. B., et al., The Making of a compound inflorescence in tomato and related nightshades. PLoS Biol, 2008. 6(11): p. e288). To begin exploring the basis for simple inflorescences like those of pepper, petunia, and tobacco, we studied a unique and previously uncharacterized tomato mutant called terminating flower (tmf), whose primary inflorescence is composed of a single flower. The tmf mutant was originally isolated from the progeny of one branch of a cv. Break o′ Day (BOD) tomato plant onto which an eggplant (Solanum melongena) scion had been grafted (Lukyanenko, A. N., E. P. Ochova, and M. Egeyan, A mutant with a single flower terminating the main stem. TGC Report, 1973. 23: p. 24).

### SUMMARY OF THE INVENTION

The present invention includes:

In one embodiment, a recombinant DNA construct comprising an isolated polynucleotide operably linked to at least one heterologous regulatory sequence, wherein the polynucleotide encodes a polypeptide having an amino acid sequence of at least 80% sequence identity, based on the Clustal W method of alignment, when compared to SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179, wherein expression of the polynucleotide in a transgenic plant can increase at least one agronomic characteristic selected from the group consisting of: leaf number, inflorescence number, branching within the inflorescence, flower number, fruit number, seed number, biomass and yield, when compared to a control plant not comprising the recombinant DNA construct. Expression of the polypeptide of part (a) in a tomato line having the tmf mutant genotype may be capable of partially or fully restoring the wild-type phenotype.

In another embodiment, a method of producing a transgenic plant with an increase of an agronomic characteristic, the method comprising: (a) introducing into a regenerable plant cell the recombinant DNA construct of claim 1 or claim 2; (b) regenerating a transgenic plant from the regenerable plant cell after step (a), wherein the transgenic plant comprises in its genome the recombinant DNA construct; and (c) selecting a transgenic plant of (b), wherein the transgenic plant comprises the recombinant DNA construct, wherein the polynucleotide is expressed, and wherein the plant exhibits an increase of at least one agronomic characteristic selected from the group consisting of: leaf number, inflorescence number, branching within the inflorescence, flower number, fruit number, seed number, biomass and yield, when compared to a control plant not comprising the recombinant DNA construct.

In another embodiment, a plant (or seed) comprising in its genome the recombinant DNA construct described herein, wherein the plant (or plant produced from the seed) exhibits an increase of at least one agronomic characteristic selected from the group consisting of: leaf number, inflorescence number, branching within the inflorescence, flower number, fruit number, seed number, biomass and yield, when compared to a control plant not comprising the recombinant DNA construct.

In another embodiment, a suppression DNA construct comprising an isolated polynucleotide operably linked, in sense or antisense orientation, to a heterologous promoter functional in a plant, wherein the polynucleotide comprises: (a) a first nucleotide sequence encoding a polypeptide having an amino acid sequence of SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179; (b) a second nucleotide sequence having at least 90% sequence identity, when compared to the first nucleotide sequence; (c) a third nucleotide sequence of at least 100 contiguous nucleotides of the first nucleotide sequence; (d) a fourth nucleotide sequence that can hybridize under stringent conditions with the first nucleotide sequence; or (e) a modified plant miRNA precursor, wherein the precursor has been modified to replace the miRNA encoding region with a sequence designed to produce a miRNA directed to the first nucleotide sequence, wherein the suppression DNA construct induces an earlier flowering time in a transgenic plant, when compared to a control plant not comprising the suppression DNA construct

In another embodiment, a method of producing a transgenic plant with an earlier flowering time, the method comprising: (a) introducing into a regenerable plant cell the suppression DNA construct described herein; (b) regenerating a transgenic plant from the regenerable plant cell after step (a), wherein the transgenic plant comprises in its genome the suppression DNA construct; and (c) selecting a transgenic plant of (b), wherein the transgenic plant comprises the suppression DNA construct and exhibits an earlier flowering time, when compared to a control plant not comprising the suppression DNA construct.

In another embodiment, a plant (or seed) comprising in its genome the suppression DNA construct described herein, wherein the plant (or plant produced from the seed) exhibits an earlier flowering time, when compared to a control plant not comprising the recombinant DNA construct.

In another embodiment, a recombinant DNA construct comprising a heterologous polynucleotide operably linked to a second polynucleotide, wherein the second polynucleotide has a nucleotide sequence selected from the group consisting of: (a) a first nucleotide sequence comprising SEQ ID NO:141; (b) a second nucleotide sequence having at least 90% sequence identity, when compared to SEQ ID NO:141; (c) a third nucleotide sequence of at least 100 contiguous nucleotides of SEQ ID NO:141; or (d) a fourth nucleotide sequence that can hybridize under stringent conditions with SEQ ID NO:141; and wherein said second polynucleotide has promoter activity in a plant.

In another embodiment, a method of expressing a heterologous polynucleotide in a plant, the method comprising: (a) transforming a regenerable plant cell with the recombinant DNA construct described above; (b) regenerating a transgenic plant from the regenerable plant cell after step (a), wherein the transgenic plant comprises in its genome the recombinant DNA construct; and (c) selecting a transgenic plant of step (b), wherein the transgenic plant comprises the recombinant DNA construct and further wherein the heterologous polynucleotide is expressed in the transgenic plant.

In another embodiment, a plant (or seed) comprising in its genome the recombinant DNA construct described above, wherein the heterologous polynucleotide is expressed in the plant (or seed, or plant produced from the seed).

In another embodiment, a method of producing a transgenic plant with an increased seed yield, the method comprising: (a) introducing into a regenerable plant cell the recombinant DNA construct of the first embodiment and the suppression DNA described above, wherein the isolated polynucleotide of the recombinant DNA construct is operably linked to a promoter functional in a plant female inflorescence tissue, and wherein the isolated polynucleotide of suppression DNA construct is operably linked, in sense or antisense orientation, to a promoter functional in a plant male inflorescence tissue; (b) regenerating a transgenic plant from the regenerable plant cell after step (a), wherein the transgenic plant comprises in its genome the suppression DNA construct and the overexpression DNA construct; and (c) selecting a transgenic plant of (b), wherein the transgenic plant comprises the recombinant DNA construct and the suppression DNA construct and exhibits an increased seed yield, when compared to a control plant not comprising the suppression DNA construct and the overexpression DNA construct.

In another embodiment, a method of producing a transgenic plant with an increased seed yield, the method comprising crossing the following: (a) a first transgenic plant comprising the recombinant DNA construct of the first embodiment, wherein the isolated polynucleotide is operably linked to a promoter functional in a plant female inflorescence tissue; with (b) a second transgenic plant comprising the suppression DNA construct of above, wherein the isolated polynucleotide is operably linked, in sense or antisense orientation, to a promoter functional in a plant male inflorescence tissue; and selecting a transgenic progeny plant of the cross, wherein the transgenic progeny plant comprises the recombinant DNA construct and the suppression DNA construct and exhibits an increased seed yield, when compared to a control plant not comprising the recombinant DNA construct and the suppression DNA construct.

In another embodiment, a plant (or seed) comprising the recombinant DNA construct of the first embodiment and the suppression DNA described above, wherein the isolated polynucleotide of the recombinant DNA construct is operably linked to a promoter functional in a plant female inflorescence tissue, and wherein the isolated polynucleotide of the suppression DNA construct is operably linked, in sense or antisense orientation, to a promoter functional in a plant male inflorescence tissue, and wherein the plant (or plant produced from the seed) has an increased seed yield.

In any of the above embodiments, the plant may be selected from the group consisting of: Arabidopsis, tomato, maize, soybean, sunflower, sorghum, canola, wheat, alfalfa, cotton, rice, barley, millet, sugar cane and switchgrass.

### BRIEF DESCRIPTION OF THE DRAWINGS AND SEQUENCE LISTING

The invention can be more fully understood from the following detailed description and the accompanying drawings and Sequence Listing which form a part of this application.

FIG. 1A-FIG. 1G show the roles of TMF in shoot organization. FIG. 1A-FIG. 1B show diagrams of the compound vegetative and inflorescence shoot systems of wild type (WT, FIG. 1A) tomato and tmf mutants (FIG. 1B). FIG. 1A shows that after the final leaf (L8) on the primary shoot meristem (PSM) terminates in the first flower (red circle), 5-6 additional flowers (orange, yellow circles) are produced by specialized axillary inflorescence meristems (SIMs). Vegetative growth continues from a sympodial vegetative meristem (SYM), which produces three leaves and initiates the next multi-flowered inflorescence. FIG. 1B shows that in tmf mutants, flowering ensues after four leaves (L4), fails to generate SIMs or SYMs, and ends growth in a single terminal flower. Canonical axillary meristems (blue arrows) are eventually released from dormancy and become typical side shoots. FIG. 1C-FIG. 1D show schematics of meristem arrangement at the floral transition in WT and tmf mutants showing canonical axillary meristems (AxM), flower meristems (FM1 and FM2), the SIM and SYM. The WT tomato cv. Break o′ Day (BOD) produces between 5-7 flowers on each regular inflorescence, while the primary tmf inflorescence is a single flower with abnormally large, leaf-like sepals. The canonical axillary meristems of tmf, and their terminal inflorescences are generally normal. FIG. 1E-FIG. 1F show scanning electron micrographs (SEM) at the flowering transition in WT (FIG. 1E) and tmf mutants (FIG. 1F). Scale bar=200 μm. Leaf and sepal numbers are as marked. FIG. 1G shows the number of leaves produced before the floral transition in wild type, a population of fully penetrant tmf mutants (tmf 100%), and a tmf line of incomplete penetrance broken down by morphological class. *=statistically significant difference from WT (p<0.05), mean+/−SD.

FIG. 2A-FIG. 2G show the cloning and expression dynamics of TMF. FIG. 2A shows the tmf mapping interval in kb. Lines indicate marker positions with the number of recombinants below. A triangle marks the position of the Rider Ty1-copia-like retrotransposon insertion. FIG. 2B shows semi-quantitative RT-PCR for TMF transcripts. FIG. 2C shows normalized read counts for the TMF gene (RPKM) across five primary meristem stages: the Early, Middle, and Late Vegetative Meristems (EVM: 5^{th} leaf initiated; MVM: 6^{th} leaf initiated; LVM: 7^{th} leaf initiated), the Transition Meristem (TM: 8^{th} leaf initiated), and the Flower Meristem (FM) and the SIM and SYM (Park, S. J., et al., Rate of meristem maturation determines inflorescence architecture in tomato. Proceedings of the National Academy of Sciences, 2012. 109(2): p. 639-644). FIG. 2C-FIG. 2F show the distribution of TMF transcripts monitored by in situ hybridization. Probes, upper right, Genotype, lower right. FIG. 2D shows that TMF is highly expressed in the EVM stage (8 days post-germination). White dashed line marks the approximate position of the cross section in FIG. 2D. FIG. 2E shows a cross section of the EVM stage showing TMF expression as a spotted ring at the periphery of the meristem and marking lateral organ boundaries. FIG. 2F shows that after the floral transition, TMF is expressed again in the SYM, which has a short vegetative phase before transitioning to the next inflorescence (FIG. 1A). FIG. 2F shows the TMF sense probe control. Scale bar=100 μm.

FIG. 3A-FIG. 3B show that overexpression of TMF promotes vegetative characteristics and branching within tomato inflorescences. FIG. 3A shows a confocal image showing nuclear localization of the 35S::GFP-TMF fusion protein. Scale bar=20 μm. FIG. 3B shows that the tmf single flower phenotype is rescued with a 35S::GFP-TMF transgene fusion and can result in overexpression phenotypes such as leaves in the inflorescence. In 5 of 6 independent 35S::GFP-TMF transgenic lines, the percentage of each population exhibiting WT phenotypes (defined as a multi-flowered inflorescence with normal sympodial shoot growth) is significantly higher than in tmf mutants (*=p-value<0.05). The ˜25% of wild type phenotypes in the tmf mutant is due to incomplete penetrance within the transformed background. Expression of 35S::GFP-TMF in the WT BOD background causes gain-of-function inflorescence defects, including ectopic formation of leaves, reversions to indeterminate vegetative shoots and branching.

FIG. 4 shows that loss of TMF drives partial and precocious activation of floral termination. A sampling of gene expression changes in tmf vegetative meristems showing that floral meristem and organ identity genes are activated precociously, while other flowering transition markers genes are unaffected.

FIG. 5A shows normalized read counts for FA, TMF, S, and AN during five stages of primary meristem maturation (Park, S. J., et al., Rate of meristem maturation determines inflorescence architecture in tomato. Proceedings of the National Academy of Sciences, 2012. 109(2): p. 639-644). FIG. 5B shows quantification of flowering time based on the number of leaves produced on the primary shoot. Unlike early flowering in tmf, fa mutants and tmf;fa double mutants flower later than WT. Columns marked with different letters are significantly different (t-test, p-value<0.05).

FIG. 6A-FIG. 6D show that the tmf lesion is a Rider Ty1-copia-like retrotransposon insertion in Solyc09g090180. FIG. 6A shows semi-quantitative RT-PCR using primers to amplify Solyc09g090180 and flanking genes from the transition meristem (TM) of tmf (left) and WT (right). Solyc9g090170 expression was not detected in either WT or tmf, and there is no evidence for expression at other stages of meristem maturation. DNA ladder is shown to the left. FIG. 6B shows that the coding region of Solyc09g090180 cannot be PCR-amplified from tmf genomic DNA, unlike a control sequence within the tmf mapping interval. 2-Log DNA ladder is shown from 500-1,000 bp. FIG. 6C shows a Southern blot probed with the complete coding sequence of Solyc09g090180 showing a genomic rearrangement in tmf mutants. For each restriction enzyme the lane order is: 1. M82 (a processing type tomato), 2. Break o′ Day (BOD: the WT progenitor of the tmf mutant), 3. tmf, 4. M99 (a fresh market tomato variety), and 5. Heinz 1706, which was sequenced by the tomato genome sequencing consortium. White arrows mark the expected fragment sizes according to the tomato genome sequence, while white asterisks highlight the structural deviation observed in tmf. FIG. 6D shows the Rider element insertion site mapping in tmf by PCR. Chromosome 9 physical positions are given in kilobases along the top. Fragments L1-L6, R1-R4 and SSR112 are amplifiable from both WT and tmf, indicating these DNA fragments are present in the mutant, whereas fragments S1-S4 can only be amplified in WT. The vertical dashed lines mark the region of genomic disturbance in tmf. The triangle marks the observed Rider transposon insertion. See Table 1 for list of PCR primers.

FIG. 7A-FIG. 7B show that the tmf-2 TILLING allele carries a mutation that disrupts a highly conserved amino acid. The tmf-2 TILLING allele is early flowering and exhibits a single flower with enlarged leaf-like sepals in the primary inflorescence like the original allele of tmf with low penetrance (8%). FIG. 7A shows a diagram of the TMF protein with the site of the missense Threonine to Isoleucine mutation in tmf-2 marked and the DUF640 domain indicated by shading. FIG. 7B shows a partial multiple sequence alignment of TMF and the predicted protein sequences of ALOG family members in Arabidopsis thaliana, Selaginella moellendorffii and Physcomitrella patens showing the highly conserved amino acid disrupted in tmf-2 and flanking residues.

FIG. 8 shows phylogenetic tree of the ALOG gene family. Maximum parsimony phylogenetic tree of the ALOG family members of tomato, Arabidopsis thaliana, Oryza sativa, Selaginella moellendorffii and Physcomitrella patens based on complete predicted protein coding sequences. Bootstrap values for 100 replicates are given to the left of nodes and At4g19500 was used as an outgroup since it is the most closely related Arabidopsis protein that is distinct from the ALOG family. The Arabidopsis ALOG family members LSH3 and LSH4 are indicated in parentheses, and are the most similar to TMF.

FIG. 9A-FIG. 9B show the multiple alignment of the amino acid sequences of the TMF polypeptides of SEQ ID NOs:46, 48, 62, 63, 65, 70, 75, 81, 88, 90, 91, 93, 100, 102, 109, 111, 116, 123 and 124. Residues that are identical to the residue of SEQ ID NO:46 at a given position are enclosed in a box. A consensus sequence is presented where a residue is shown if identical in all sequences, otherwise, a period is shown.

FIG. 10 shows the percent sequence identity and the divergence values for each pair of amino acids sequences of TMF polypeptides displayed in FIG. 9A-FIG. 9B.

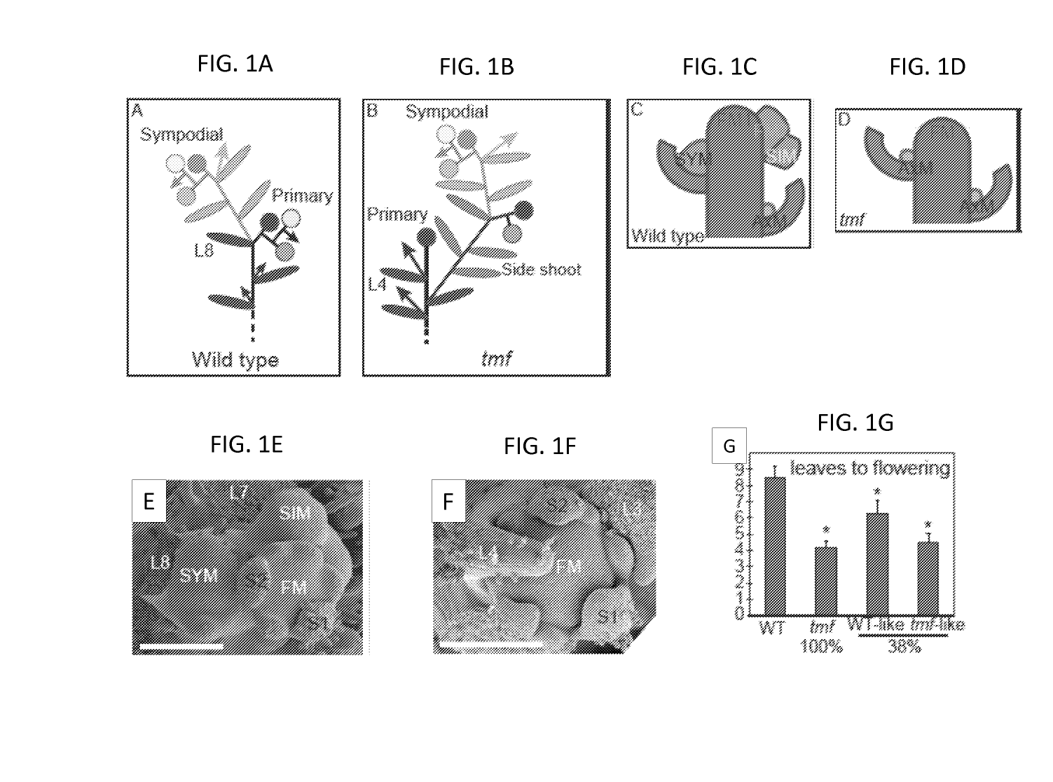

Primers used in this study are shown in Table 1 below.

TABLE 1
Sequencing Primers

| Prime Name | Sequence 5′ to 3′ | SEQ ID NO |
|---|---|---|
| Solyc09g090180 seqf | AAATAGTAATAATAGGG | 1 |
| | AAAAATAGGG | |
| Solyc09g090180 seqr | ACCTCTCTTCTCTCTCT | 2 |
| | CCC | |
| Solyc09g090160 f | AAATAAATACAGAGGAA | 3 |
| | AATTTTTGC | |
| Solyc09g090160 r | TCTGCATATGCGTTTAC | 4 |
| | TGC | |
| Solyc09g090160 seq | AGAAAGCCTTTCAGGTT | 5 |
| | GG | |
| Solyc09g090130 f | CATAAAAAAATAAAAAA | 6 |
| | ATTCATCAAAG | |
| Solyc09g090130 seqr | AATTGTTTGAACTTTTCA | 7 |
| | AGGC | |
| Solyc09g090170 f | GAAATATTCCAATAATA | 8 |
| | ATTTGGACC | |
| Solyc09g090170 f | TTCTTCTCAAACCATTTA | 9 |
| | ATTCC | |

Primers for Mapping the tmf Recombination Breakpoint

| | | | For |
|---|---|---|---|
| Prime Name | Sequence 5′ to 3′ | SEQ ID NO | Fragment |
| SSR112f | GGAACACAACCAAGAA | 10 | S1, S2, S4 |
| | GTGGA | | |
| SSR112r | TATCGGCTTAGGGTTGT | 11 | R4 |
| | TGG | | |
| 514.7f | GACATGATTCTACATAG | 12 | R1 |
| | GAGG | | |
| 514.7r | GACCACAAAAAACAAGA | 13 | R1 |
| | CTGC | | |
| 63.1 f | AAATAAATACAGAGGAA | 14 | L1 |
| | AATTTTTGC | | |
| 63.1 r | TCTGCATATGCGTTTAC | 15 | L1 |
| | TGC | | |
| zf anchor f | TGTACCACTTTAAATTT | 16 | L2 |
| | GTGATGC | | |
| zf anchor r | TCAAACAAACAAAATGA | 17 | L2 |
| | CGC | | |
| a fragment f | ACTTGTTCACCGTTCAA | 18 | L3 |
| | ACG | | |
| a fragment r | ATTTATGATTATGTGGA | 19 | L3 |
| | TCAAACC | | |
| ATG+ 1600 | GAACACCCTGAAACATT | 20 | S2 |
| | TCC | | |
| ATG+ 2100 | GGCAAGCCAATTATGTA | 21 | S1 |
| | TACC | | |
| a-b for | ACAACCAAACAACCCTT | 22 | L4, S4 |
| | TGC | | |
| a-b rev | TACCGTTACTTGGTCAC | 23 | L4 |
| | TCC | | |
| c fragment f | ATTCTTGGACTAGACTC | 24 | R3 |
| | TGC | | |
| c fragment r | CCTTTTCACACTACCCT | 25 | R3 & R4 |
| | TCG | | |
| d fragment f | AAAGGTCATGGAGACAT | 26 | R2 |
| | ACC | | |
| d fragment r | CAGACGTAACGTTAACA | 27 | R2 |
| | TCG | | |
| TMF seqf | AAATAGTAATAATAGGG | 28 | S3 |
| | AAAAATAGGG | | |
| TMF seqr | ACCTCTCTTCTCTCTCT | 29 | S3 |
| | CCC | | |
| Control for gDNA f | GAAATATTCCAATAATA | 30 | |
| | ATTTGGACC | | |
| Control for gDNA r | TTCTTCTCAAACCATTTA | 31 | |
| | ATTCC | | |
| hi-TAIL 0 | GGTTCTTATAACCTACT | 32 | hi-TAIL |
| | CCCTAGCTCCTCTATTA | | PCR |
| | CCC | | |
| hi-TAIL 1 | ACGATGGACTCCAGTC | 33 | |
| | CGGCCTCTCTCCCAAAT | | |
| | AAAAGATCATCAAATCG | | |
| hi-TAIL 2 | TAACAATTCGATGACGA | 34 | |
| | TGTTAGCGG | | |

RT-PCT Primers

| Primer Name | Sequence 5′ to 3′ | SEQ ID NO |
|---|---|---|
| Solyc09g090190 rtf | TTTTGTACCTGGTCAAC | 35 |
| | TAAGC | |
| Solyc09g090190 rtr | GAGAATAAGGTTATACG | 36 |
| | TTTTGAGG | |
| Ubiquitin f | CGTGGTGGTGCTAAGA | 37 |
| | AGAG | |
| Ubiquitin r | ACGAAGCCTCTGAACCT | 38 |
| | TTC | |

Primers for Cloning, RT-PCR, in situ and Southern Probe Synthesis

| Primer Name | Sequence 5′ to 3′ | SEQ ID NO |
|---|---|---|
| TMFgs | caccATGGAACACAACCA | 39 |
| | AGAAGTGG | |
| TMFr | TTAGCTTGAATTTCCAT | 40 |
| | TTGG | |

Primers for Generation of pS::LhG4 and pOp::AN

| Primer Name | Sequence 5′ to 3′ | SEQ ID NO |
|---|---|---|
| pS PstI-F | AAACTGCAGCTATCAAG | 41 |
| | GATTTTTCAA | |
| pS BamHI-R | AAAGGATCCATTTGATG | 42 |
| | AGGATGAAGAAG | |
| ANCDS XhoI-F | AAACTCGAGATGGAAG | 43 |
| | CTTTTCATCATCC | |
| ANCDS KpnI-R | AAAGGTACCTCAGTTGA | 44 |
| | ATGACTGAAAGG | |

SEQ ID NO:45 is the nucleotide sequence corresponding to the protein-coding region of the TMF gene.

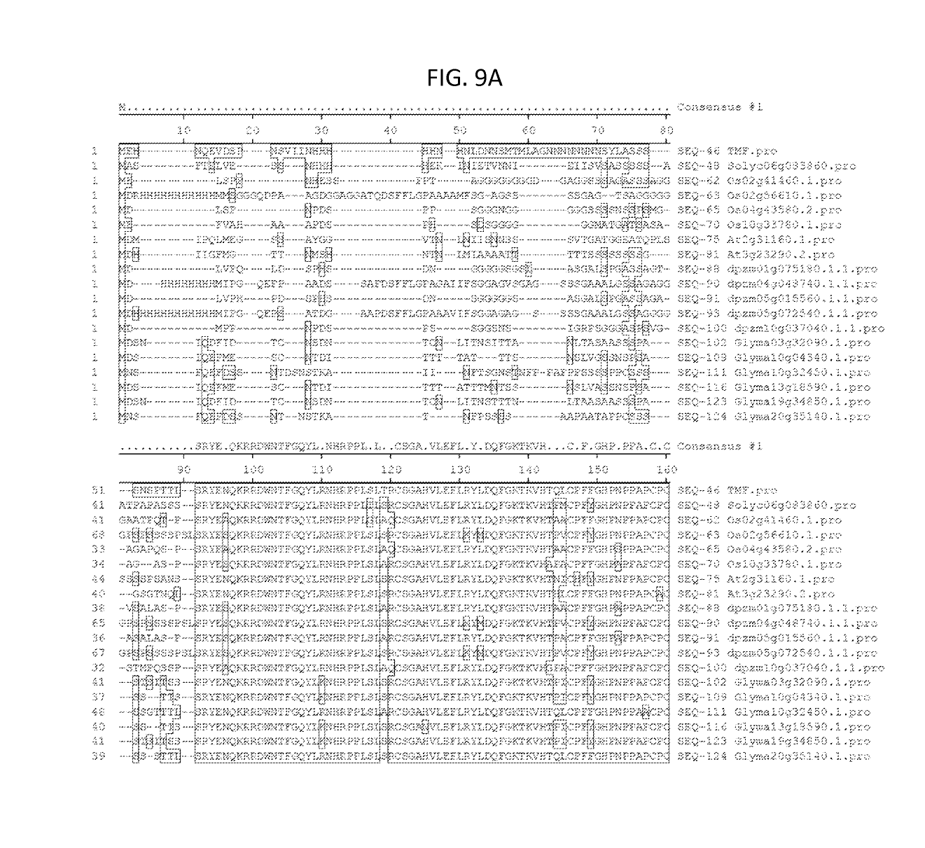

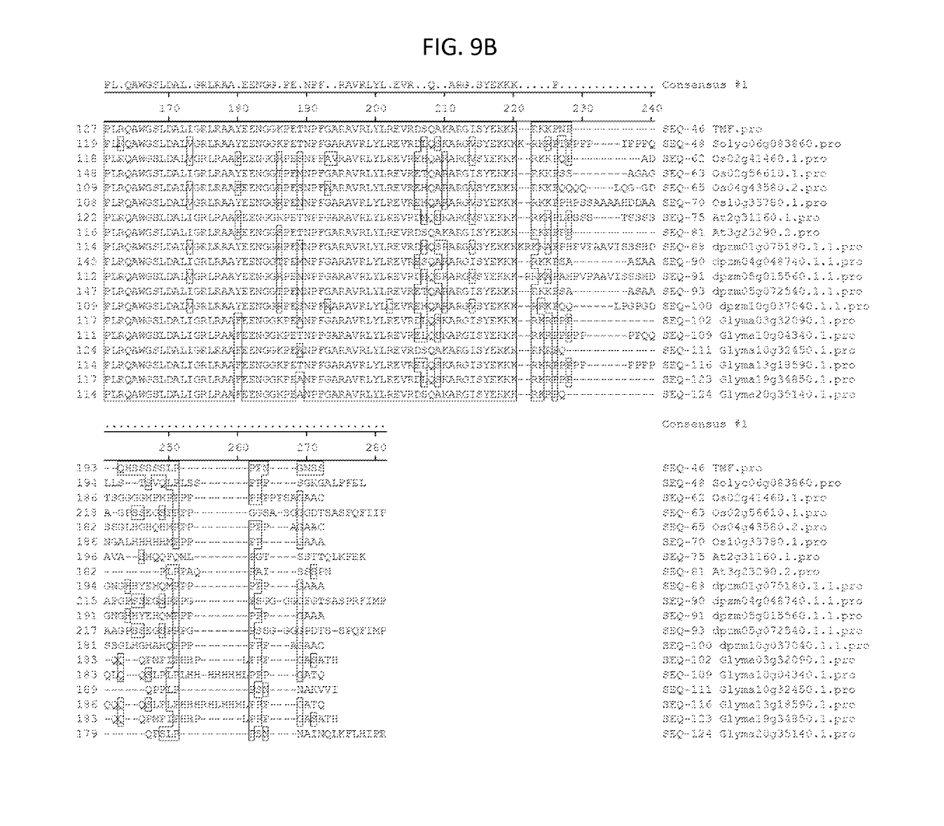

Amino acid sequence for members of the ALOG gene family from various species are shown in Tables 2 and 3 below. Table 2 presents the amino acid sequences used in creating a phylogentic tree of the ALOG gene family members (FIG. 9).

TABLE 2
Amino Acid Sequences in Phylogenetic Tree of the ALOG Gene Family

| SPECIES | GENE NAME | SEQ ID NO |
|---|---|---|
| Solanum lycopersicum | Solyc09g090180 (TMF) | 46 |
| Solanum lycopersicum | Solyc09g025280 | 47 |
| Solanum lycopersicum | Solyc06g083860 | 48 |
| Solanum lycopersicum | Solyc06g082210 | 49 |
| Solanum lycopersicum | Solyc02g069510 | 50 |
| Solanum lycopersicum | Solyc05g055020 | 51 |
| Solanum lycopersicum | Solyc02g076820 | 52 |
| Solanum lycopersicum | Solyc12g014260 | 53 |
| Solanum lycopersicum | Solyc04g009980 | 54 |
| Solanum lycopersicum | Solyc10g007310 | 55 |
| Solanum lycopersicum | Solyc10g008000 | 56 |
| Solanum lycopersicum | Solyc07g062470 | 57 |
| Selaginella moellendorffii | Smo: 36560 | 58 |
| Selaginella moellendorffii | Smo: 68566 | 59 |
| Oryza sativa | Os01g54180.1 | 60 |
| Oryza sativa | Os01g61310.1 | 61 |
| Oryza sativa | Os02g41460.1 | 62 |
| Oryza sativa | Os02g56610.1 | 63 |
| Oryza sativa | Os02g07030.3 | 64 |
| Oryza sativa | Os04g43580.2 | 65 |
| Oryza sativa | Os05g28040.1 | 66 |
| Oryza sativa | Os05g39500.1 | 67 |
| Oryza sativa | Os06g46030.1 | 68 |
| Oryza sativa | Os07g04670.1 | 69 |
| Oryza sativa | Os10g33780.1 | 70 |
| Physcomitrella patens | Pp1s44_284V6.1 | 71 |
| Physcomitrella patens | Pp1s73_217V6.3 | 72 |
| Physcomitrella patens | Pp1s10_72V6.2 | 73 |
| Physcomitrella patens | Pp1s8_54V6.1 | 74 |
| Arabidopsis thaliana | At2g31160.1 (LSH3) | 75 |
| Arabidopsis thaliana | At2g42610.1 (LSH10) | 76 |
| Arabidopsis thaliana | At4g18610.1 (LSH9) | 77 |
| Arabidopsis thaliana | At1g78815.1 (LSH7) | 78 |
| Arabidopsis thaliana | At1g07090.1 (LSH6) | 79 |
| Arabidopsis thaliana | At1g16910.1 (LSH8) | 80 |
| Arabidopsis thaliana | At3g23290.2 (LSH4) | 81 |
| Arabidopsis thaliana | At3g04510.1 (LSH2) | 82 |
| Arabidopsis thaliana | At5g28490.1 (LSH1) | 83 |
| Arabidopsis thaliana | At5g58500.1 (LSH5) | 84 |
| Arabidopsis thaliana | At4g19500 (Outgroup) | 85 |

TABLE 3
Amino Acid Sequences of ALOG Proteins from
Maize, Soybean and Sorghum

| SPECIES | GENE NAME | SEQ ID NO |
|---|---|---|
| Zea mays | dpzm00g102743.0.1 | 86 |
| Zea mays | dpzm00g104795.0.1 | 87 |
| Zea mays | dpzm01g075180.1.1 | 88 |
| Zea mays | dpzm03g050540.1.1 | 89 |
| Zea mays | dpzm04g048740.1.1 | 90 |
| Zea mays | dpzm05g015560.1.1 | 91 |
| Zea mays | dpzm05g032380.1.3 | 92 |
| Zea mays | dpzm05g072540.1.1 | 93 |
| Zea mays | dpzm06g025480.1.1 | 94 |
| Zea mays | dpzm06g043310.1.1 | 95 |
| Zea mays | dpzm07g002720.1.1 | 96 |
| Zea mays | dpzm08g031330.1.1 | 97 |
| Zea mays | dpzm08g032360.1.1 | 98 |
| Zea mays | dpzm08g055580.1.1 | 99 |
| Zea mays | dpzm10g037040.1.1 | 100 |
| Glycine max | Glyma02g39570.1 | 101 |
| Glycine max | Glyma03g32090.1 | 102 |
| Glycine max | Glyma03g35600.1 | 103 |
| Glycine max | Glyma03g41140.1 | 104 |
| Glycine max | Glyma05g25310.1 | 105 |
| Glycine max | Glyma06g43040.1 | 106 |
| Glycine max | Glyma08g08320.1 | 107 |
| Glycine max | Glyma08g12250.1 | 108 |
| Glycine max | Glyma10g04340.1 | 109 |
| Glycine max | Glyma10g30890.1 | 110 |
| Glycine max | Glyma10g32450.1 | 111 |
| Glycine max | Glyma11g29340.1 | 112 |
| Glycine max | Glyma12g15250.1 | 113 |
| Glycine max | Glyma12g33840.1 | 114 |
| Glycine max | Glyma12g35850.1 | 115 |
| Glycine max | Glyma13g18590.1 | 116 |
| Glycine max | Glyma13g30290.1 | 117 |
| Glycine max | Glyma13g34550.1 | 118 |
| Glycine max | Glyma13g36660.1 | 119 |
| Glycine max | Glyma14g37650.1 | 120 |
| Glycine max | Glyma15g08880.1 | 121 |
| Glycine max | Glyma18g06590.1 | 122 |
| Glycine max | Glyma19g34850.1 | 123 |
| Glycine max | Glyma20g35140.1 | 124 |
| Glycine max | Glyma20g36580.1 | 125 |
| Sorghum bicolor | Sb01g019290.1 | 126 |
| Sorghum bicolor | Sb02g002650.1 | 127 |
| Sorghum bicolor | Sb03g038670.1 | 128 |
| Sorghum bicolor | Sb03g045430.1 | 129 |
| Sorghum bicolor | Sb04g004470.1 | 130 |
| Sorghum bicolor | Sb04g026450.1 | 131 |
| Sorghum bicolor | Sb04g036620.1 | 132 |
| Sorghum bicolor | Sb06g022610.1 | 133 |
| Sorghum bicolor | Sb09g016440.1 | 134 |
| Sorghum bicolor | Sb09g023120.1 | 135 |
| Sorghum bicolor | Sb10g027020.1 | 136 |

SEQ ID NO:137 is the nucleotide sequence of the attB1 site.

SEQ ID NO:138 is the nucleotide sequence of the attB2 site.

SEQ ID NO:139 is the nucleotide sequence of the VC062 primer, containing the T3 promoter and attB1 site, useful to amplify cDNA inserts cloned into a BLUESCRIPT® II SK(+) vector (Stratagene).

SEQ ID NO:140 is the nucleotide sequence of the VC063 primer, containing the T7 promoter and attB2 site, useful to amplify cDNA inserts cloned into a BLUESCRIPT® II SK(+) vector (Stratagene).

SEQ ID NO:141 is the nucleotide sequence of the TMF promoter.

SEQ ID NO:142 is the nucleotide sequence of the coding region for ANANTHA from tomato (Lycopersicon esculentum).

SEQ ID NO:143 is the nucleotide sequence of the octopine synthase (OCS) transcription terminator from Agrobacterium tumefaciens.

TABLE 4
Sequences of ALOG Proteins

| | | SEQ ID NO: | SEQ ID NO: |
|---|---|---|---|
| SPECIES | GENE NAME | (nucleotide) | (amino acid) |
| Sesbania bispinosa | sesgr1n.pk132.a12 | 144 | 145 |
| Sesbania bispinosa | sesgr1n.pk111.a5 | 146 | 147 |
| Sesbania bispinosa | sesgr1n.pk134.m18 | 148 | 149 |
| Sesbania bispinosa | sesgr1n.pk069.d21 | 150 | 151 |
| Amaranthus | ahgr1c.pk159.c2 | 152 | 153 |
| hypochondriacus | | | |
| Artemisia tridentata | arttr1n.pk139.e22 | 154 | 155 |
| Artemisia tridentata | arttr1n.pk018.f23 | 156 | 157 |
| Artemisia tridentata | arttr1n.pk099.l16 | 158 | 159 |
| Artemisia tridentata | arttr1n.pk166.i7 | 160 | 161 |
| Artemisia tridentata | arttr1n.pk121.k22 | 162 | 163 |
| Artemisia tridentata | arttr1n.pk215.o3 | 164 | 165 |
| Lamium amplexicaule | hengr1n.pk010.h24 | 166 | 167 |
| Lamium amplexicaule | hengr1n.pk045.a4 | 168 | 169 |
| Peperomia caperata | pepgr1n.pk126.p16 | 170 | 171 |
| Eschscholzia | ecalgr1n.pk027.g21 | 172 | 173 |
| californica | | | |
| Eschscholzia | ecalgr1n.pk047.l20 | 174 | 175 |
| californica | | | |
| Linum perenne | lpgr1n.pk110.g3 | 176 | 177 |
| Linum perenne | lpgr1n.pk119.g1 | 178 | 179 |

The sequence descriptions and Sequence Listing attached hereto comply with the rules governing nucleotide and/or amino acid sequence disclosures in patent applications as set forth in 37 C.F.R. §1.821-1.825.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

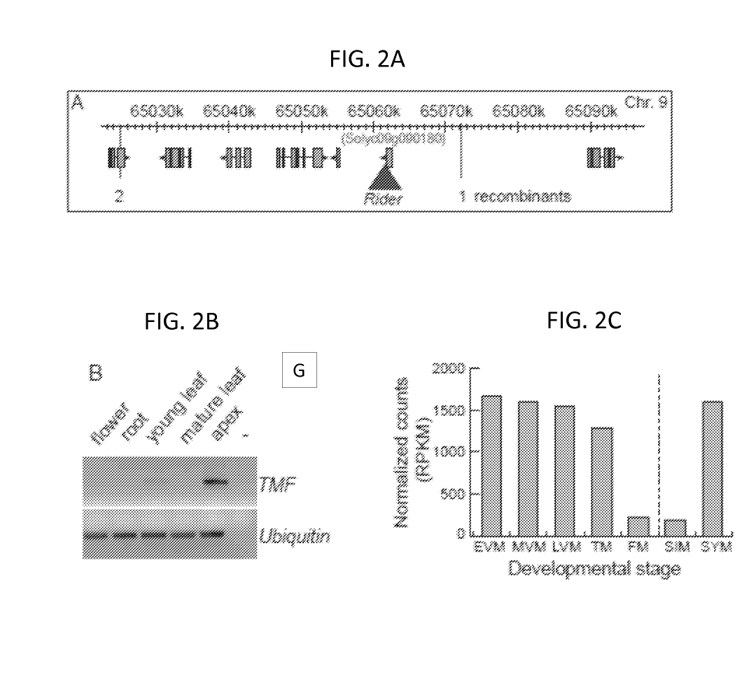

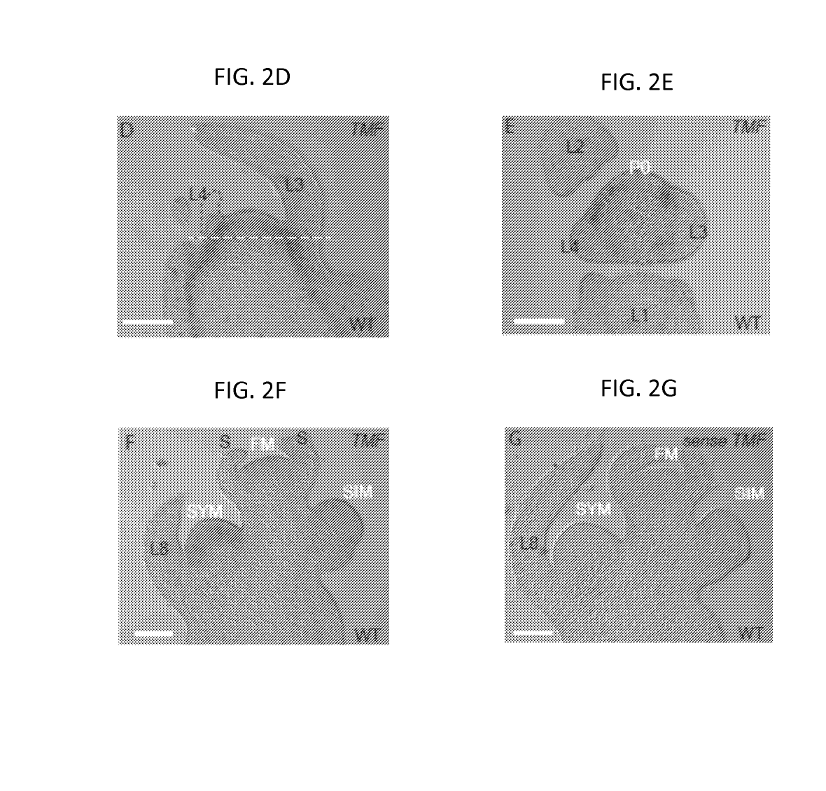

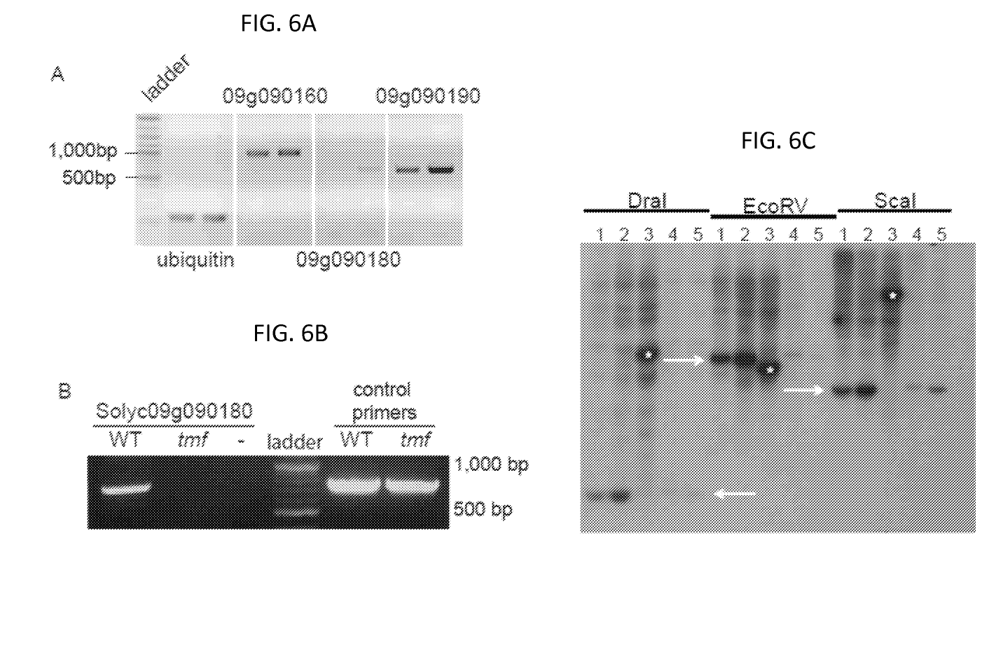

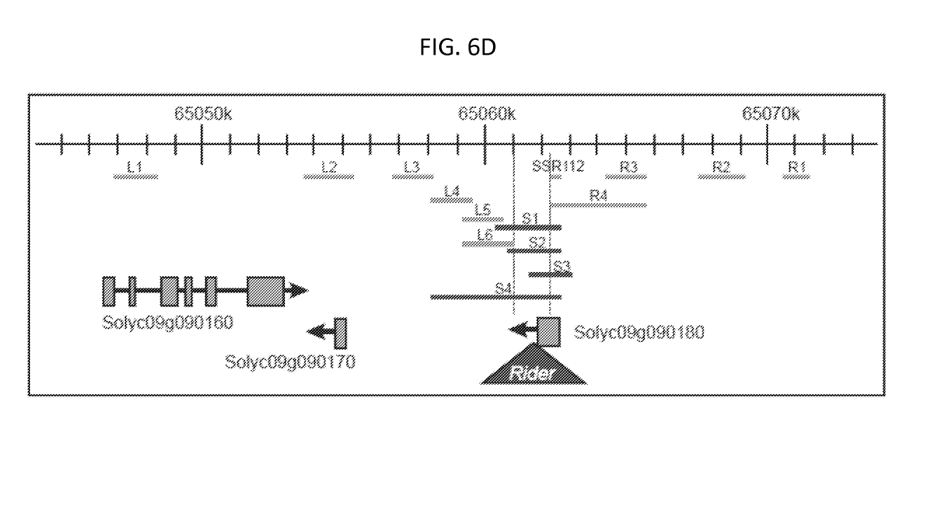

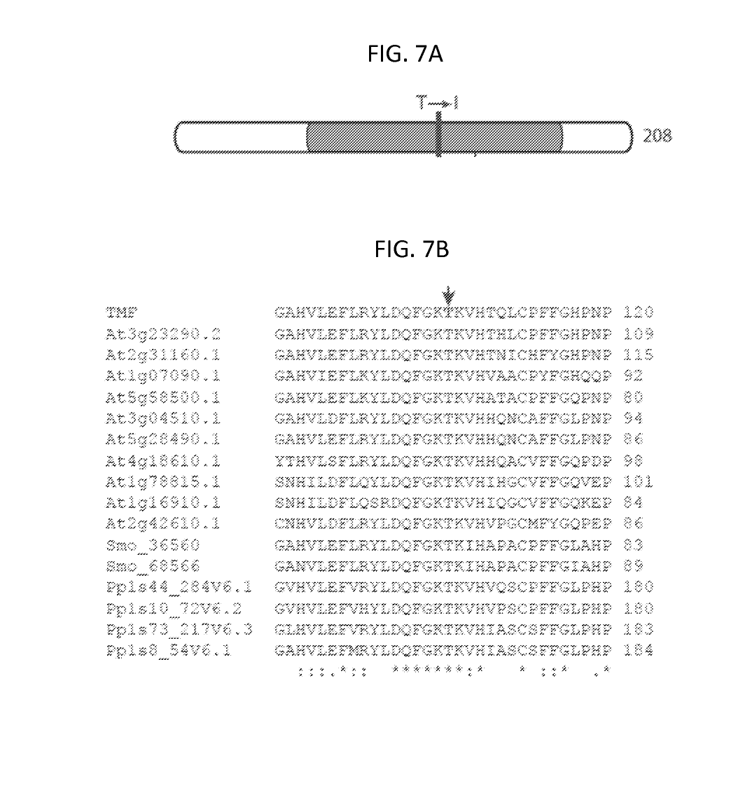

The tmf mutant was originally isolated from the progeny of one branch of a cv. Break o'Day (BOD) tomato plant onto which an eggplant (S. melongena) scion had been grafted (Lukyanenko, A. N., E. P. Ochova, and M. Egeyan, A mutant with a single flower terminating the main stem. TGC Report, 1973. 23: p. 24). To identify the causative mutation, we mapped tmf to a 47 kb region on chromosome 9, and of the five genes in the mapping interval (FIG. 2A), Solyc09g090180, annotated as a small nuclear protein of unknown function, was expressed in WT apices, but not in mutants (FIG. 6A). To confirm that Solyc09g090180 encodes TMF, we isolated a second allele (tmf-2) by TILLING an ethane methyl sulfonate (EMS) mutagenized population (Menda, N., et al., In silico screening of a saturated mutation library of tomato. Plant J, 2004. 38(5): p. 861-72; Henikoff, S., B. J. Till, and L. Comai, TILLING. Traditional Mutagenesis Meets Functional Genomics. Plant Physiology, 2004. 135(2): p. 630-636). The tmf-2 lesion is a missense mutation that converts a highly conserved threonine to an isoleucine (FIGS. 7A and 7B).

As used herein, a polypeptide (or polynucleotide) with “TMF activity” refers to a polypeptide (or polynucleotide), that when expressed in a tmf mutant line, is capable of partially or fully rescuing the tmf phenotype. The term “TMF polypeptide” refers to a polypeptide with TMF activity.

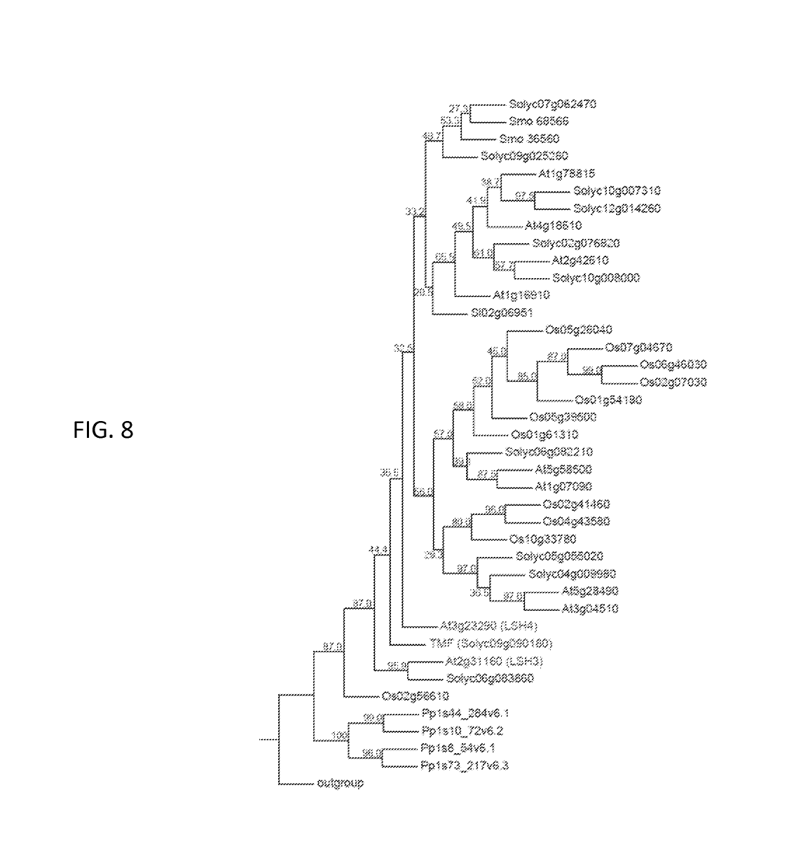

TMF encodes a member of the ALOG (Arabidopsis Light Sensitive Hypocotyl 1, Oryza G1) gene family of nuclear localized proteins containing a single strongly conserved central domain of unknown function (DUF640; Bateman, A., et al., The Pfam protein families database. Nucleic Acids Research, 2002. 30(1): p. 276-280) with little other sequence homology (Zhao, L., et al., Overexpression of LSH1, a member of an uncharacterised gene family, causes enhanced light regulation of seedling development. The Plant Journal, 2004. 37(5): p. 694-706; Yoshida, A., et al., The homeotic gene long sterile lemma (G1) specifies sterile lemma identity in the rice spikelet. Proceedings of the National Academy of Sciences of the United States of America, 2009. 106(47): p. 20103-8). The tomato genome encodes 11 additional ALOG genes, similar to the 10 member Arabidopsis (Zhao, L., et al., Overexpression of LSH1, a member of an uncharacterised gene family, causes enhanced light regulation of seedling development. The Plant Journal, 2004. 37(5): p. 694-706) and rice (Yoshida, A., et al., The homeotic gene long sterile lemma (G1) specifies sterile lemma identity in the rice spikelet. Proceedings of the National Academy of Sciences of the United States of America, 2009. 106(47): p. 20103-8) families (FIG. 8).

The terms “monocot” and “monocotyledonous plant” are used interchangeably herein. A monocot of the current invention includes the Gramineae.

The terms “dicot” and “dicotyledonous plant” are used interchangeably herein. A dicot of the current invention includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield.

“Agronomic characteristic” is a measurable parameter including but not limited to: leaf number, inflorescence number, branching within the inflorescence, flower number, fruit number, seed number, biomass, yield, flowering time, greenness, growth rate, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress.

Increased biomass can be measured, for example, as an increase in plant height, plant total leaf area, plant fresh weight, plant dry weight or plant seed yield, as compared with control plants.

The ability to increase the biomass or size of a plant would have several important commercial applications. Crop species may be generated that produce larger cultivars, generating higher yield in, for example, plants in which the vegetative portion of the plant is useful as food, biofuel or both.

Increased leaf size may be of particular interest. Increasing leaf biomass can be used to increase production of plant-derived pharmaceutical or industrial products. An increase in total plant photosynthesis is typically achieved by increasing leaf area of the plant. Additional photosynthetic capacity may be used to increase the yield derived from particular plant tissue, including the leaves, roots, fruits or seed, or permit the growth of a plant under decreased light intensity or under high light intensity.

Modification of the biomass of another tissue, such as root tissue, may be useful to improve a plant's ability to grow under harsh environmental conditions, including drought or nutrient deprivation, because larger roots may better reach water or nutrients or take up water or nutrients.

For some ornamental plants, the ability to provide larger varieties would be highly desirable. For many plants, including fruit-bearing trees, trees that are used for lumber production, or trees and shrubs that serve as view or wind screens, increased stature provides improved benefits in the forms of greater yield or improved screening.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, plant propagules, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Propagule” includes all products of meiosis and mitosis able to propagate a new plant, including but not limited to, seeds, spores and parts of a plant that serve as a means of vegetative reproduction, such as corms, tubers, offsets, or runners. Propagule also includes grafts where one portion of a plant is grafted to another portion of a different plant (even one of a different species) to create a living organism. Propagule also includes all plants and seeds produced by cloning or by bringing together meiotic products, or allowing meiotic products to come together to form an embryo or fertilized egg (naturally or with human intervention).

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different transgenes.

“Transgenic plant” also includes reference to plants which comprise more than one heterologous polynucleotide within their genome. Each heterologous polynucleotide may confer a different trait to the transgenic plant.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” refers to the RNA that is without introns and that can be translated into protein by the cell.

“cDNA” refers to a DNA that is complementary to and synthesized from a mRNA template using the enzyme reverse transcriptase. The cDNA can be single-stranded or converted into the double-stranded form using the Klenow fragment of DNA polymerase I.

“Coding region” refers to the portion of a messenger RNA (or the corresponding portion of another nucleic acid molecule such as a DNA molecule) which encodes a protein or polypeptide. “Non-coding region” refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

“Mature” protein refers to a post-translationally processed polypeptide; i.e., one from which any pre- or pro-peptides present in the primary translation product have been removed.

“Precursor” protein refers to the primary product of translation of mRNA; i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature.

The terms “entry clone” and “entry vector” are used interchangeably herein.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably, and refer to a promoter that is expressed predominantly but not necessarily exclusively in one tissue or organ, but that may also be expressed in one specific cell.

“Developmentally regulated promoter” refers to a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

A “chloroplast transit peptide” is an amino acid sequence which is translated in conjunction with a protein and directs the protein to the chloroplast or other plastid types present in the cell in which the protein is made (Lee et al. (2008) Plant Cell 20:1603-1622). The terms “chloroplast transit peptide” and “plastid transit peptide” are used interchangeably herein. “Chloroplast transit sequence” refers to a nucleotide sequence that encodes a chloroplast transit peptide. A “signal peptide” is an amino acid sequence which is translated in conjunction with a protein and directs the protein to the secretory system (Chrispeels (1991) Ann. Rev. Plant Phys. Plant Mol. Biol. 42:21-53). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel (1992) Plant Phys. 100:1627-1632). A “mitochondrial signal peptide” is an amino acid sequence which directs a precursor protein into the mitochondria (Zhang and Glaser (2002) Trends Plant Sci 7:14-21). Sequence alignments and percent identity calculations may be determined using a variety of comparison methods designed to detect homologous sequences including, but not limited to, the Megalign® program of the LASERGENE® bioinformatics computing suite (DNASTAR® Inc., Madison, Wis.).

The Clustal W method of alignment may be used to compare sequences. The Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program. Unless stated otherwise, percent identities and divergences provided and claimed herein were calculated in this manner.

Alternatively, multiple alignment can be performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

Turning now to the embodiments:

Embodiments include isolated polynucleotides and polypeptides, recombinant DNA constructs, compositions (such as plants or seeds) comprising these recombinant DNA constructs, and methods utilizing these recombinant DNA constructs.

Isolated Polynucleotides and Polypeptides:

The present invention includes the following isolated polynucleotides and polypeptides:

An isolated polynucleotide comprising: (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal W method of alignment, when compared to SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179; or (ii) a full complement of the nucleic acid sequence of (i), wherein the full complement and the nucleic acid sequence of (i) consist of the same number of nucleotides and are 100% complementary. Any of the foregoing isolated polynucleotides may be utilized in any recombinant DNA constructs (including suppression DNA constructs) of the present invention. The polypeptide preferably has TMF activity.

An isolated polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal W method of alignment, when compared to SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179. The polypeptide preferably has TMF activity

An isolated polynucleotide comprising (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal W method of alignment, when compared to a second polynucleotide encoding a polypeptide with an amino acid sequence of SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179; or (ii) a full complement of the nucleic acid sequence of (i). Any of the foregoing isolated polynucleotides may be utilized in any recombinant DNA constructs (including suppression DNA constructs) of the present invention. The isolated polynucleotide preferably encodes a polypeptide with TMF activity. The nucleotide sequence of the second polynucleotide may be SEQ ID NO:45.

An isolated polynucleotide comprising a nucleotide sequence, wherein the nucleotide sequence is hybridizable under stringent conditions with a DNA molecule comprising the full complement of a second polynucleotide encoding a polypeptide with an amino acid sequence of SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179. The isolated polynucleotide preferably encodes a polypeptide with TMF activity. The nucleotide sequence of the second polynucleotide may be SEQ ID NO:45.

An isolated polynucleotide comprising a nucleotide sequence, wherein the nucleotide sequence is derived from a second polynucleotide encoding a polypeptide with an amino acid sequence of SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179 by alteration of one or more nucleotides by at least one method selected from the group consisting of: deletion, substitution, addition and insertion. The isolated polynucleotide preferably encodes a polypeptide with TMF activity. The nucleotide sequence of the second polynucleotide may be SEQ ID NO:45.

An isolated polynucleotide comprising a nucleotide sequence, wherein the nucleotide sequence corresponds to an allele of a second polynucleotide encoding a polypeptide with an amino acid sequence of SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179. The nucleotide sequence of the second polynucleotide may be SEQ ID NO:45.

It is understood, as those skilled in the art will appreciate, that the invention encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

The protein of the current invention may also be a protein which comprises an amino acid sequence comprising deletion, substitution, insertion and/or addition of one or more amino acids in an amino acid sequence presented in SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179. The substitution may be conservative, which means the replacement of a certain amino acid residue by another residue having similar physical and chemical characteristics. Non-limiting examples of conservative substitution include replacement between aliphatic group-containing amino acid residues such as Ile, Val, Leu or Ala, and replacement between polar residues such as Lys-Arg, Glu-Asp or Gln-Asn replacement.

Proteins derived by amino acid deletion, substitution, insertion and/or addition can be prepared when DNAs encoding their wild-type proteins are subjected to, for example, well-known site-directed mutagenesis (see, e.g., Nucleic Acid Research, Vol. 10, No. 20, p. 6487-6500, 1982, which is hereby incorporated by reference in its entirety). As used herein, the term “one or more amino acids” is intended to mean a possible number of amino acids which may be deleted, substituted, inserted and/or added by site-directed mutagenesis.

Site-directed mutagenesis may be accomplished, for example, as follows using a synthetic oligonucleotide primer that is complementary to single-stranded phage DNA to be mutated, except for having a specific mismatch (i.e., a desired mutation). Namely, the above synthetic oligonucleotide is used as a primer to cause synthesis of a complementary strand by phages, and the resulting duplex DNA is then used to transform host cells. The transformed bacterial culture is plated on agar, whereby plaques are allowed to form from phage-containing single cells. As a result, in theory, 50% of new colonies contain phages with the mutation as a single strand, while the remaining 50% have the original sequence. At a temperature which allows hybridization with DNA completely identical to one having the above desired mutation, but not with DNA having the original strand, the resulting plaques are allowed to hybridize with a synthetic probe labeled by kinase treatment. Subsequently, plaques hybridized with the probe are picked up and cultured for collection of their DNA.

Techniques for allowing deletion, substitution, insertion and/or addition of one or more amino acids in the amino acid sequences of biologically active peptides such as enzymes while retaining their activity include site-directed mutagenesis mentioned above, as well as other techniques such as those for treating a gene with a mutagen, and those in which a gene is selectively cleaved to remove, substitute, insert or add a selected nucleotide or nucleotides, and then ligated.

The protein of the present invention may also be a protein which is encoded by a nucleic acid comprising a nucleotide sequence comprising deletion, substitution, insertion and/or addition of one or more nucleotides in the nucleotide sequence of a polynucleotide encoding a polypeptide with an amino acid sequence of SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179. Nucleotide deletion, substitution, insertion and/or addition may be accomplished by site-directed mutagenesis or other techniques as mentioned above.

The protein of the present invention may also be a protein which is encoded by a nucleic acid comprising a nucleotide sequence hybridizable under stringent conditions with the complementary strand of the nucleotide sequence of a polynucleotide encoding a polypeptide with an amino acid sequence of SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179. The nucleotide sequence of the polynucleotide may be SEQ ID NO:45.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system (Amersham). Stringent conditions include, for example, hybridization at 42° C. for 4 hours using the hybridization buffer included in the kit, which is supplemented with 5% (w/v) Blocking reagent and 0.5 M NaCl, and washing twice in 0.4% SDS, 0.5×SSC at 55° C. for 20 minutes and once in 2×SSC at room temperature for 5 minutes.

Recombinant DNA Constructs and Suppression DNA Constructs:

In one aspect, the present invention includes recombinant DNA constructs (including suppression DNA constructs).

In one embodiment, a recombinant DNA construct comprises a polynucleotide operably linked to at least one regulatory sequence (e.g., a promoter functional in a plant), wherein the polynucleotide comprises (i) a nucleic acid sequence encoding an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal W method of alignment, when compared to SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179; or (ii) a full complement of the nucleic acid sequence of (i).

In another embodiment, a recombinant DNA construct comprises a polynucleotide operably linked to at least one regulatory sequence (e.g., a promoter functional in a plant), wherein said polynucleotide comprises (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal W method of alignment, when compared to a second polynucleotide encoding a polypeptide with an amino acid sequence of SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179; or (ii) a full complement of the nucleic acid sequence of (i). The nucleotide sequence of the second polynucleotide may be SEQ ID NO:45.

In another embodiment, a recombinant DNA construct comprises a polynucleotide operably linked to at least one regulatory sequence (e.g., a promoter functional in a plant), wherein said polynucleotide encodes a polypeptide having TMF activity. The polypeptide with TMF activity may be from, but is not limited to, the following: Arabidopsis thaliana, Zea mays, Glycine max, Glycine tabacina, Glycine soja, Glycine tomentella, Oryza sativa, Brassica napus, Sorghum bicolor, Saccharum officinarum or Triticum aestivum.

In any of the recombinant DNA constructs described herein, the polynucleotide may be operably linked to at least one heterologous regulatory sequence

In another aspect, the present invention includes suppression DNA constructs.

A suppression DNA construct may comprise at least one regulatory sequence (e.g., a promoter functional in a plant) operably linked to (a) all or part of: (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal W method of alignment, when compared to SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179, or (ii) a full complement of the nucleic acid sequence of (a)(i); or (b) a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal W method of alignment, when compared to said all or part of a sense strand or antisense strand from which said region is derived, and wherein said target gene of interest encodes a TMF polypeptide; or (c) all or part of: (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal W method of alignment, when compared to a polynucleotide encoding a polypeptide with an amino acid sequence of SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179, or (ii) a full complement of the nucleic acid sequence of (c)(i). The nucleotide sequence of the polynucleotide of (c)(i) may be SEQ ID NO:45. The suppression DNA construct may comprise a cosuppression construct, antisense construct, viral-suppression construct, hairpin suppression construct, stem-loop suppression construct, double-stranded RNA-producing construct, RNAi construct, or small RNA construct (e.g., an siRNA construct or an miRNA construct).

In any of the suppression DNA constructs described herein, the polynucleotide may be operably linked to at least one heterologous regulatory sequence

It is understood, as those skilled in the art will appreciate, that the invention encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the sense strand (or antisense strand) of the gene of interest.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as sRNA (short interfering RNA) constructs and miRNA (microRNA) constructs.

“Antisense inhibition” refers to the production of antisense RNA transcripts capable of suppressing the expression of the target gene or gene product. “Antisense RNA” refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns, or the coding sequence.

“Cosuppression” refers to the production of sense RNA transcripts capable of suppressing the expression of the target gene or gene product. “Sense” RNA refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see Vaucheret et al., Plant J. 16:651-659 (1998); and Gura, Nature 404:804-808 (2000)).

Another variation describes the use of plant viral sequences to direct the suppression of proximal mRNA encoding sequences (PCT Publication No. WO 98/36083 published on Aug. 20, 1998).

RNA interference refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)).

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants (Lagos-Quintana et al., Science 294:853-858 (2001), Lagos-Quintana et al., Curr. Biol. 12:735-739 (2002); Lau et al., Science 294:858-862 (2001); Lee and Ambros, Science 294:862-864 (2001); Llave et al., Plant Cell 14:1605-1619 (2002); Mourelatos et al., Genes Dev. 16:720-728 (2002); Park et al., Curr. Biol. 12:1484-1495 (2002); Reinhart et al., Genes. Dev. 16:1616-1626 (2002)). They are processed from longer precursor transcripts that range in size from approximately 70 to 200 nt, and these precursor transcripts have the ability to form stable hairpin structures.

MicroRNAs (miRNAs) appear to regulate target genes by binding to complementary sequences located in the transcripts produced by these genes. It seems likely that miRNAs can enter at least two pathways of target gene regulation: (1) translational inhibition; and (2) RNA cleavage. MicroRNAs entering the RNA cleavage pathway are analogous to the 21-25 nt short interfering RNAs (siRNAs) generated during RNA interference (RNAi) in animals and posttranscriptional gene silencing (PTGS) in plants, and likely are incorporated into an RNA-induced silencing complex (RISC) that is similar or identical to that seen for RNAi.

In one embodiment, there is provided a method for the suppression of a target sequence comprising introducing into a cell a nucleic acid construct encoding a miRNA substantially complementary to the target. In some embodiments the miRNA comprises about 19, 20, 21, 22, 23, 24 or 25 nucleotides. In some embodiments the miRNA comprises 21 nucleotides. In some embodiments the nucleic acid construct encodes the miRNA. In some embodiments the nucleic acid construct encodes a polynucleotide precursor which may form a double-stranded RNA, or hairpin structure comprising the miRNA.

In some embodiments, the nucleic acid construct comprises a modified endogenous plant miRNA precursor, wherein the precursor has been modified to replace the endogenous miRNA encoding region with a sequence designed to produce a miRNA directed to the target sequence. The plant miRNA precursor may be full-length of may comprise a fragment of the full-length precursor. In some embodiments, the endogenous plant miRNA precursor is from a dicot or a monocot. In some embodiments the endogenous miRNA precursor is from Arabidopsis, tomato, maize, soybean, sunflower, sorghum, canola, wheat, alfalfa, cotton, rice, barley, millet, sugar cane or switchgrass.

In some embodiments, the miRNA template, (i.e. the polynucleotide encoding the miRNA), and thereby the miRNA, may comprise some mismatches relative to the target sequence. In some embodiments the miRNA template has >1 nucleotide mismatch as compared to the target sequence, for example, the miRNA template can have 1, 2, 3, 4, 5, or more mismatches as compared to the target sequence. This degree of mismatch may also be described by determining the percent identity of the miRNA template to the complement of the target sequence. For example, the miRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the target sequence.

In some embodiments, the miRNA template, (i.e. the polynucleotide encoding the miRNA) and thereby the miRNA, may comprise some mismatches relative to the miRNA backside. In some embodiments the miRNA template has >1 nucleotide mismatch as compared to the miRNA backside, for example, the miRNA template can have 1, 2, 3, 4, 5, or more mismatches as compared to the miRNA backside. This degree of mismatch may also be described by determining the percent identity of the miRNA template to the complement of the miRNA backside. For example, the miRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the miRNA backside.

Regulatory Sequences:

A recombinant DNA construct (including a suppression DNA construct) of the present invention may comprise at least one regulatory sequence.

A regulatory sequence may be heterologous.

A regulatory sequence may be a promoter.

A number of promoters can be used in recombinant DNA constructs of the present invention. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High level, constitutive expression of the candidate gene under control of the 35S or UBI promoter may have pleiotropic effects, although gene efficacy may be estimated when driven by a constitutive promoter. Use of tissue-specific and/or inducible promoters may eliminate undesirable effects.

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)); ALS promoter (U.S. Pat. No. 5,659,026), the constitutive synthetic core promoter SCP1 (International Publication No. 03/033651) and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the invention, it may be desirable to use a tissue-specific or developmentally regulated promoter.

A tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter may be used in the methods of the present invention which causes the desired temporal and spatial expression.

Promoters which are seed or embryo-specific and may be useful in the invention include soybean Kunitz trypsin inhibitor (Kti3, Jofuku and Goldberg, Plant Cell 1:1079-1093 (1989)), patatin (potato tubers) (Rocha-Sosa, M., et al. (1989) EMBO J. 8:23-29), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. U.S.A. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564), and sporamin (sweet potato tuberous root) (Hattori, T., et al. (1990) Plant Mol. Biol. 14:595-604). Promoters of seed-specific genes operably linked to heterologous coding regions in chimeric gene constructions maintain their temporal and spatial expression pattern in transgenic plants. Such examples include Arabidopsis thaliana 2S seed storage protein gene promoter to express enkephalin peptides in Arabidopsis and Brassica napus seeds (Vanderkerckhove et al., Bio/Technology 7:L929-932 (1989)), bean lectin and bean beta-phaseolin promoters to express luciferase (Riggs et al., Plant Sci. 63:47-57 (1989)), and wheat glutenin promoters to express chloramphenicol acetyl transferase (Colot et al., EMBO J 6:3559-3564 (1987)).

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Promoters for use in the current invention include the following: 1) the stress-inducible RD29A promoter (Kasuga et al. (1999) Nature Biotechnol. 17:287-91); 2) the barley promoter, B22E; expression of B22E is specific to the pedicel in developing maize kernels (“Primary Structure of a Novel Barley Gene Differentially Expressed in Immature Aleurone Layers”. Klemsdal, S. S. et al., Mol. Gen. Genet. 228(1/2):9-16 (1991)); and 3) maize promoter, Zag2 (“Identification and molecular characterization of ZAG1, the maize homolog of the Arabidopsis floral homeotic gene AGAMOUS”, Schmidt, R. J. et al., Plant Cell 5(7):729-737 (1993); “Structural characterization, chromosomal localization and phylogenetic evaluation of two pairs of AGAMOUS-like MADS-box genes from maize”, Theissen et al. Gene 156(2):155-166 (1995); NCBI GenBank Accession No. X80206)). Zag2 transcripts can be detected 5 days prior to pollination to 7 to 8 days after pollination (“DAP”), and directs expression in the carpel of developing female inflorescences and Ciml which is specific to the nucleus of developing maize kernels. Ciml transcript is detected 4 to 5 days before pollination to 6 to 8 DAP. Other useful promoters include any promoter which can be derived from a gene whose expression is maternally associated with developing female florets.

Additional promoters for regulating the expression of the nucleotide sequences of the present invention in plants are stalk-specific promoters. Such stalk-specific promoters include the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

Promoters may be derived in their entirety from a native gene, or be composed of different elements derived from different promoters found in nature, or even comprise synthetic DNA segments.

Promoters for use in the current invention may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (Genbank accession number EF030817), and the constitutive promoter GOS2 from Zea mays. Other promoters include root preferred promoters, such as the maize NAS2 promoter, the maize Cyclo promoter (US 2006/0156439, published Jul. 13, 2006), the maize ROOTMET2 promoter (WO05063998, published Jul. 14, 2005), the CR1B10 promoter (WO06055487, published May 26, 2006), the CRWAQ81 (WO05035770, published Apr. 21, 2005) and the maize ZRP2.47 promoter (NCBI accession number: U38790; GI No. 1063664),

Recombinant DNA constructs of the present invention may also include other regulatory sequences, including but not limited to, translation leader sequences, introns, and polyadenylation recognition sequences. In another embodiment of the present invention, a recombinant DNA construct of the present invention further comprises an enhancer or silencer.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, Mol. Cell Biol. 8:4395-4405 (1988); Callis et al., Genes Dev. 1:1183-1200 (1987).

Any plant can be selected for the identification of regulatory sequences and TMF genes to be used in recombinant DNA constructs and other compositions (e.g. transgenic plants, seeds and cells) and methods of the present invention. Examples of suitable plants for the isolation of genes and regulatory sequences and for compositions and methods of the present invention would include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassava, castorbean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugarcane, sunflower, sweet potato, sweetgum, switchgrass, tangerine, tea, tobacco, tomato, triticale, turf, turnip, a vine, watermelon, wheat, yams, and zucchini.

A composition of the present invention includes a transgenic microorganism, cell, plant, and seed comprising the recombinant DNA construct. The cell may be eukaryotic, e.g., a yeast, insect or plant cell, or prokaryotic, e.g., a bacterial cell. The transgenic microorganism may be Agrobacterium, e.g., Agrobacterium tumefaciens or Agrobacterium rhizogenes.

A composition of the present invention is a plant comprising in its genome any of the recombinant DNA constructs (including any of the suppression DNA constructs) of the present invention (such as any of the constructs discussed above). Compositions also include any progeny of the plant, and any seed obtained from the plant or its progeny, wherein the progeny or seed comprises within its genome the recombinant DNA construct (or suppression DNA construct). Progeny includes subsequent generations obtained by self-pollination or out-crossing of a plant. Progeny also includes hybrids and inbreds.

In hybrid seed propagated crops, mature transgenic plants can be self-pollinated to produce a homozygous inbred plant. The inbred plant produces seed containing the newly introduced recombinant DNA construct (or suppression DNA construct). These seeds can be grown to produce plants that would exhibit an altered agronomic characteristic (e.g., an increased agronomic characteristic optionally under water limiting conditions), or used in a breeding program to produce hybrid seed, which can be grown to produce plants that would exhibit such an altered agronomic characteristic. The seeds may be maize seeds.

The plant may be a monocotyledonous or dicotyledonous plant, for example, a maize or soybean plant, such as a maize hybrid plant or a maize inbred plant. The plant may also be sunflower, sorghum, canola, wheat, alfalfa, cotton, rice, barley, millet, sugar cane or switchgrass.

The recombinant DNA construct may be stably integrated into the genome of the plant.

Particular embodiments include but are not limited to the following:

In an embodiment, a method of producing a transgenic plant with an increase of an agronomic characteristic, the method comprising: (a) introducing into a regenerable plant cell a recombinant DNA construct comprising an isolated polynucleotide operably linked to at least one regulatory sequence, wherein the polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal W method of alignment, when compared to SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179; (b) regenerating a transgenic plant from the regenerable plant cell after step (a), wherein the transgenic plant comprises in its genome the recombinant DNA construct; and (c) selecting a transgenic plant of (b), wherein the transgenic plant comprises the recombinant DNA construct and exhibits an increase of at least one agronomic characteristic selected from the group consisting of: leaf number, inflorescence number, branching within the inflorescence, flower number, fruit number, seed number, biomass and yield, when compared to a control plant not comprising the recombinant DNA construct. Expression of the polypeptide of part (a) in a tomato line having the tmf mutant genotype may be capable of partially or fully restoring the wild-type phenotype.

In another embodiment, a plant comprising in its genome a recombinant DNA construct comprising an isolated polynucleotide operably linked to at least one regulatory sequence, wherein the polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal W method of alignment, when compared to SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179, and wherein the plant exhibits an increase of at least one agronomic characteristic selected from the group consisting of: leaf number, inflorescence number, branching within the inflorescence, flower number, fruit number, seed number, biomass and yield, when compared to a control plant not comprising the recombinant DNA construct.

In another embodiment, seed of the plant of above, wherein said seed comprises in its genome a recombinant DNA construct comprising a polynucleotide operably linked to at least one regulatory element, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal W method of alignment, when compared to SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179, and wherein a plant produced from the seed exhibits an increase of at least one agronomic characteristic selected from the group consisting of: leaf number, inflorescence number, branching within the inflorescence, flower number, fruit number, seed number, biomass and yield, when compared to a control plant not comprising the recombinant DNA construct.

In another embodiment, a method of producing a transgenic plant with an earlier flowering time, the method comprising: (a) introducing into a regenerable plant cell a suppression DNA construct comprising an isolated polynucleotide operably linked, in sense or antisense orientation, to a promoter functional in a plant, wherein the polynucleotide comprises: (i) a first nucleotide sequence encoding a polypeptide having an amino acid sequence of SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179; (ii) a second nucleotide sequence having at least 90% sequence identity, when compared to the first nucleotide sequence; (iii) a third nucleotide sequence of at least 100 contiguous nucleotides of the first nucleotide sequence; (iv) a fourth nucleotide sequence that can hybridize under stringent conditions with the first nucleotide sequence, and optionally is derived from the first nucleotide sequence by alteration of one or more nucleotides by at least one method selected from the group consisting of: deletion, substitution, addition and insertion; or (v) a modified plant miRNA precursor, wherein the precursor has been modified to replace the miRNA encoding region with a sequence designed to produce a miRNA directed to the first nucleotide sequence; (b) regenerating a transgenic plant from the regenerable plant cell after step (a), wherein the transgenic plant comprises in its genome the recombinant DNA construct; and (c) selecting a transgenic plant of (b), wherein the transgenic plant comprises the recombinant DNA construct and exhibits an earlier flowering time, when compared to a control plant not comprising the recombinant DNA construct. The first nucleotide sequence may be SEQ ID NO:45.

In another embodiment, a plant comprising in its genome a recombinant DNA construct comprising an isolated polynucleotide operably linked, in sense or antisense orientation or both, to a promoter functional in a plant, wherein the polynucleotide comprises: (i) a first nucleotide sequence encoding a polypeptide having an amino acid sequence of SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179; (ii) a second nucleotide sequence having at least 90% sequence identity, when compared to the first nucleotide sequence; (iii) a third nucleotide sequence of at least 100 contiguous nucleotides of the first nucleotide sequence; (iv) a fourth nucleotide sequence that can hybridize under stringent conditions with the first nucleotide sequence, and optionally is derived from the first nucleotide sequence by alteration of one or more nucleotides by at least one method selected from the group consisting of: deletion, substitution, addition and insertion; or (v) a modified plant miRNA precursor, wherein the precursor has been modified to replace the miRNA encoding region with a sequence designed to produce a miRNA directed to the first nucleotide sequence; and wherein the plant exhibits an earlier flowering time, when compared to a control plant not comprising the recombinant DNA construct. The first nucleotide sequence may be SEQ ID NO:45.

In another embodiment, seed of the plant of above, wherein said seed comprises in its genome a recombinant DNA construct comprising an isolated polynucleotide operably linked, in sense or antisense orientation, to a promoter functional in a plant, wherein the polynucleotide comprises: (i) a first nucleotide sequence encoding a polypeptide having an amino acid sequence of SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179; (ii) a second nucleotide sequence having at least 90% sequence identity, when compared to the first nucleotide sequence; (iii) a third nucleotide sequence of at least 100 contiguous nucleotides of the first nucleotide sequence; or (iv) a fourth nucleotide sequence that can hybridize under stringent conditions with the first nucleotide sequence, and optionally is derived from the first nucleotide sequence by alteration of one or more nucleotides by at least one method selected from the group consisting of: deletion, substitution, addition and insertion; or (v) a modified plant miRNA precursor, wherein the precursor has been modified to replace the miRNA encoding region with a sequence designed to produce a miRNA directed to the first nucleotide sequence; and wherein a plant produced from the seed exhibits an earlier flowering time, when compared to a control plant not comprising the recombinant DNA construct. The first nucleotide sequence may be SEQ ID NO:45.

In another embodiment, a method of expressing a heterologous polynucleotide in a plant, the method comprising: (a) transforming a regenerable plant cell with a recombinant DNA construct comprising a heterologous polynucleotide operably linked to a second polynucleotide, wherein the second polynucleotide has a nucleotide sequence selected from the group consisting of: (i) a first nucleotide sequence comprising SEQ ID NO:141; (ii) a second nucleotide sequence having at least 90% sequence identity, when compared to SEQ ID NO:141; (iii) a third nucleotide sequence of at least 100 contiguous nucleotides of SEQ ID NO:141; or (iv) a fourth nucleotide sequence that can hybridize under stringent conditions with SEQ ID NO:141, and optionally is derived from the first nucleotide sequence by alteration of one or more nucleotides by at least one method selected from the group consisting of: deletion, substitution, addition and insertion; and (b) regenerating a transgenic plant from the regenerable plant cell after step (a), wherein the transgenic plant comprises in its genome the recombinant DNA construct; and (c) selecting a transgenic plant of (b), wherein the transgenic plant comprises the recombinant DNA construct and further wherein the heterologous polynucleotide is expressed in the transgenic plant.

In another embodiment, a plant comprising in its genome a recombinant DNA construct comprising a heterologous polynucleotide operably linked to a second polynucleotide, wherein the second polynucleotide has a nucleotide sequence selected from the group consisting of: (i) a first nucleotide sequence comprising SEQ ID NO:141; (ii) a second nucleotide sequence having at least 90% sequence identity, when compared to SEQ ID NO:141; (iii) a third nucleotide sequence of at least 100 contiguous nucleotides of SEQ ID NO:141; or (iv) a fourth nucleotide sequence that can hybridize under stringent conditions with SEQ ID NO:141, and optionally is derived from the first nucleotide sequence by alteration of one or more nucleotides by at least one method selected from the group consisting of: deletion, substitution, addition and insertion; and wherein the heterologous polynucleotide is expressed in the plant.

In another embodiment, seed of the plant of above, wherein said seed comprises in its genome a recombinant DNA construct comprising a heterologous polynucleotide operably linked to a second polynucleotide, wherein the second polynucleotide has a nucleotide sequence selected from the group consisting of: (i)

a first nucleotide sequence comprising SEQ ID NO:141; (ii) a second nucleotide sequence having at least 90% sequence identity, when compared to SEQ ID NO:141; (iii) a third nucleotide sequence of at least 100 contiguous nucleotides of SEQ ID NO:141; or (iv) a fourth nucleotide sequence that can hybridize under stringent conditions with SEQ ID NO:141, and optionally is derived from the first nucleotide sequence by alteration of one or more nucleotides by at least one method selected from the group consisting of: deletion, substitution, addition and insertion; and wherein the hetereologous polynucleotide is expressed in a plant produced from the seed.

In any of the above embodiments, the plant may be selected from, but is not limited to, the group consisting of: Arabidopsis, tomato, maize, soybean, sunflower, sorghum, canola, wheat, alfalfa, cotton, rice, barley, millet, sugar cane and switchgrass.

In another embodiment, a method of producing a transgenic plant with an increased seed yield, the method comprising: (a) introducing into a regenerable plant cell a suppression DNA construct and an overexpression DNA construct, wherein the suppression DNA construct comprises an isolated polynucleotide operably linked, in sense or antisense orientation, to a promoter functional in a plant male inflorescence tissue, wherein the polynucleotide comprises: (i) a first nucleotide sequence encoding a polypeptide having an amino acid sequence of SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179; (ii) a second nucleotide sequence having at least 90% sequence identity, when compared to the first nucleotide sequence; (iii) a third nucleotide sequence of at least 100 contiguous nucleotides of the first nucleotide sequence; (iv) a fourth nucleotide sequence that can hybridize under stringent conditions with the first nucleotide sequence, and optionally is derived from the first nucleotide sequence by alteration of one or more nucleotides by at least one method selected from the group consisting of: deletion, substitution, addition and insertion; or (v) a modified plant miRNA precursor, wherein the precursor has been modified to replace the miRNA encoding region with a sequence designed to produce a miRNA directed to the first nucleotide sequence; and wherein the overexpression DNA construct comprises an isolated polynucleotide operably linked to a promoter functional in a plant female inflorescence tissue, wherein the polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal W method of alignment, when compared to SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179; (b) regenerating a transgenic plant from the regenerable plant cell after step (a), wherein the transgenic plant comprises in its genome the suppression DNA construct and the overexpression DNA construct; and (c) selecting a transgenic plant of (b), wherein the transgenic plant comprises the suppression DNA construct and the overexpression DNA construct and exhibits an increased seed yield, when compared to a control plant not comprising the suppression DNA construct and the overexpression DNA construct. The plant may be selected from the group consisting of: maize, rice, wheat, sorghum and canola. The first nucleotide sequence may be SEQ ID NO:45.

In another embodiment, a method of producing a transgenic plant with an increased seed yield, the method comprising crossing the following: (a) a first transgenic plant comprising a suppression DNA construct, wherein the suppression DNA construct comprises an isolated polynucleotide operably linked, in sense or antisense orientation, to a promoter functional in a plant male inflorescence tissue, wherein the polynucleotide comprises: (i) a first nucleotide sequence encoding a polypeptide having an amino acid sequence of SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179; (ii) a second nucleotide sequence having at least 90% sequence identity, when compared to the first nucleotide sequence; (iii) a third nucleotide sequence of at least 100 contiguous nucleotides of the first nucleotide sequence; (iv) fourth nucleotide sequence that can hybridize under stringent conditions with the first nucleotide sequence, and optionally is derived from the first nucleotide sequence by alteration of one or more nucleotides by at least one method selected from the group consisting of: deletion, substitution, addition and insertion; or (v) a modified plant miRNA precursor, wherein the precursor has been modified to replace the miRNA encoding region with a sequence designed to produce a miRNA directed to the first nucleotide sequence; with (b) a second transgenic plant comprising an overexpression DNA construct, wherein the overexpression DNA construct comprises an isolated polynucleotide operably linked to a promoter functional in a plant female inflorescence tissue, wherein the polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal W method of alignment, when compared to SEQ ID NO:46, 48, 62, 63, 65, 70, 75, 81, 88, 90, 91, 93, 100, 102, 109, 111, 116, 123, 124, 145, 147, 149, 151, 153, 155, 157, 159, 161, 163, 165, 167, 169, 171, 173, 175, 177 or 179; and selecting a transgenic progeny plant of the cross, wherein the transgenic progeny plant comprises the suppression DNA construct and the overexpression DNA construct and exhibits an increased seed yield, when compared to a control plant not comprising the suppression DNA construct and overexpression DNA construct. The plant may be selected from the group consisting of: maize, rice, wheat, sorghum and canola. The first nucleotide sequence of the first transgenic plant may be SEQ ID NO:45.

In another embodiment, any progeny of the plants in the embodiments described herein, any seeds of the plants in the embodiments described herein, any seeds of progeny of the plants in embodiments described herein, and cells from any of the above plants in embodiments described herein and progeny thereof.

In any of the embodiments described herein, the TMF polypeptide may be from Arabidopsis thaliana, Zea mays, Glycine max, Glycine tabacina, Glycine soja, Glycine tomentella, Oryza sativa, Brassica napus, Sorghum bicolor, Saccharum officinarum, or Triticum aestivum.

In any of the embodiments described herein, the recombinant DNA construct (or suppression DNA construct) may comprise a polynucleotide operably linked to at least one heterologous regulatory sequence

In any of the embodiments described herein, the recombinant DNA construct (or suppression DNA construct) may comprise at least a promoter functional in a plant as a regulatory sequence. The promoter may be heterologous.

In any of the embodiments described herein, the plant may exhibit increased yield, for example, at least 5%, at least 10%, at least 15%, at least 20% or at least 25% increased yield, relative to the control plants.

Typically, when a transgenic plant comprising a recombinant DNA construct or suppression DNA construct in its genome exhibits increased yield relative to a reference or control plant, the reference or control plant does not comprise in its genome the recombinant DNA construct or suppression DNA construct.

One of ordinary skill in the art would readily recognize a suitable control or reference plant to be utilized when assessing or measuring an agronomic characteristic or phenotype of a transgenic plant in any embodiment of the present invention in which a control plant is utilized (e.g., compositions or methods as described herein). For example, by way of non-limiting illustrations:

1. Progeny of a transformed plant which is hemizygous with respect to a recombinant DNA construct (or suppression DNA construct), such that the progeny are segregating into plants either comprising or not comprising the recombinant DNA construct (or suppression DNA construct): the progeny comprising the recombinant DNA construct (or suppression DNA construct) would be typically measured relative to the progeny not comprising the recombinant DNA construct (or suppression DNA construct) (i.e., the progeny not comprising the recombinant DNA construct (or the suppression DNA construct) is the control or reference plant).

2. Introgression of a recombinant DNA construct (or suppression DNA construct) into an inbred line, such as in maize, or into a variety, such as in soybean: the introgressed line would typically be measured relative to the parent inbred or variety line (i.e., the parent inbred or variety line is the control or reference plant).

3. Two hybrid lines, where the first hybrid line is produced from two parent inbred lines, and the second hybrid line is produced from the same two parent inbred lines except that one of the parent inbred lines contains a recombinant DNA construct (or suppression DNA construct): the second hybrid line would typically be measured relative to the first hybrid line (i.e., the first hybrid line is the control or reference plant).

4. A plant comprising a recombinant DNA construct (or suppression DNA construct): the plant may be assessed or measured relative to a control plant not comprising the recombinant DNA construct (or suppression DNA construct) but otherwise having a comparable genetic background to the plant (e.g., sharing at least 90%, 91%, 92%, 93%, 94%, 95%, 96%, 97%, 98%, 99%, or 100% sequence identity of nuclear genetic material compared to the plant comprising the recombinant DNA construct (or suppression DNA construct)). There are many laboratory-based techniques available for the analysis, comparison and characterization of plant genetic backgrounds; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLP®s), and Simple Sequence Repeats (SSRs) which are also referred to as Microsatellites.

Furthermore, one of ordinary skill in the art would readily recognize that a suitable control or reference plant to be utilized when assessing or measuring an agronomic characteristic or phenotype of a transgenic plant would not include a plant that had been previously selected, via mutagenesis or transformation, for the desired agronomic characteristic or phenotype.

In another embodiment, a method for transforming a cell (or microorganism) comprising transforming a cell (or microorganism) with any of the isolated polynucleotides or recombinant DNA constructs of the present invention. The cell (or microorganism) transformed by this method is also included. In particular embodiments, the cell is eukaryotic cell, e.g., a yeast, insect or plant cell, or prokaryotic, e.g., a bacterial cell. The microorganism may be Agrobacterium, e.g. Agrobacterium tumefaciens or Agrobacterium rhizogenes.

A method for producing a transgenic plant comprising transforming a plant cell with any of the isolated polynucleotides or recombinant DNA constructs (including suppression DNA constructs) of the present invention and regenerating a transgenic plant from the transformed plant cell. The invention is also directed to the transgenic plant produced by this method, and transgenic seed obtained from this transgenic plant. The transgenic plant obtained by this method may be used in other methods of the present invention.

A method for isolating a polypeptide of the invention from a cell or culture medium of the cell, wherein the cell comprises a recombinant DNA construct comprising a polynucleotide of the invention operably linked to at least one regulatory sequence, and wherein the transformed host cell is grown under conditions that are suitable for expression of the recombinant DNA construct.

A method of altering the level of expression of a polypeptide of the invention in a host cell comprising: (a) transforming a host cell with a recombinant DNA construct of the present invention; and (b) growing the transformed host cell under conditions that are suitable for expression of the recombinant DNA construct wherein expression of the recombinant DNA construct results in production of altered levels of the polypeptide of the invention in the transformed host cell.

A method of producing seed (for example, seed that can be sold as a commercial product offering) comprising any of the preceding methods, and further comprising obtaining seeds from said progeny plant, wherein said seeds comprise in their genome said recombinant DNA construct (or suppression DNA construct).

In any of the preceding methods or any other embodiments of methods of the present invention, in said introducing step said regenerable plant cell may comprise a callus cell, an embryogenic callus cell, a gametic cell, a meristematic cell, or a cell of an immature embryo. The regenerable plant cells may derive from an inbred maize plant.

In any of the preceding methods or any other embodiments of methods of the present invention, said regenerating step may comprise the following: (i) culturing said transformed plant cells in a media comprising an embryogenic promoting hormone until callus organization is observed; (ii) transferring said transformed plant cells of step (i) to a first media which includes a tissue organization promoting hormone; and (iii) subculturing said transformed plant cells after step (ii) onto a second media, to allow for shoot elongation, root development or both.

In any of the preceding methods or any other embodiments of methods of the present invention, alternatives exist for introducing into a regenerable plant cell a recombinant DNA construct comprising a polynucleotide operably linked to at least one regulatory sequence. For example, one may introduce into a regenerable plant cell a regulatory sequence (such as one or more enhancers, optionally as part of a transposable element), and then screen for an event in which the regulatory sequence is operably linked to an endogenous gene encoding a polypeptide of the instant invention.

The introduction of recombinant DNA constructs of the present invention into plants may be carried out by any suitable technique, including but not limited to direct DNA uptake, chemical treatment, electroporation, microinjection, cell fusion, infection, vector-mediated DNA transfer, bombardment, or Agrobacterium-mediated transformation. Techniques for plant transformation and regeneration have been described in International Patent Publication WO 2009/006276, the contents of which are herein incorporated by reference.

The development or regeneration of plants containing the foreign, exogenous isolated nucleic acid fragment that encodes a protein of interest is well known in the art. The regenerated plants may be self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

### EXAMPLES

The present invention is further illustrated in the following Examples, in which parts and percentages are by weight and degrees are Celsius, unless otherwise stated. It should be understood that these Examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

### The Primary Meristem of tmf Mutants Precociously Terminates

The compound shoots of tomato originate from developmentally distinct types of meristems, and their initiation and development (maturation) is temporally and spatially coordinated with the flowering transition (Pnueli, L., et al., The SELF-PRUNING gene of tomato regulates vegetative to reproductive switching of sympodial meristems and is the ortholog of CEN and TFL1. Development, 1998. 125(11): p. 1979-89; Lippman, Z. B., et al., The Making of a compound inflorescence in tomato and related nightshades. PLoS Biol, 2008. 6(11): p. e288; Lifschitz, E., et al., The tomato FT ortholog triggers systemic signals that regulate growth and flowering and substitute for diverse environmental stimuli. Proc Natl Acad Sci USA, 2006. 103(16): p. 6398-403). In wild type (WT) domesticated tomatoes, the primary shoot meristem (PSM) produces 7-12 leaves before switching to reproductive growth and terminating in a flower. At the base of this flower, a meristem initiates and quickly transitions into a flower itself, but not before giving rise to a new meristem at its base. These meristems are known as “sympodial inflorescence meristems” (SIM), and their rapid termination and reiteration forms a multi-flowered zigzag inflorescence. Vegetative growth then continues from the axillary meristem formed in the axil of the uppermost leaf on the PSM, and this “sympodial vegetative meristem” (SYM) generates three leaves in a shortened vegetative phase before also transitioning to a flower. A new SYM forms in the axil of the last leaf produced by each prior SYM, and this process reiterates to produce a compound vegetative shoot. In contrast, the delayed growth of canonical axillary meristems hosted by lower leaves recapitulates the production of the primary shoot before switching into regular sympodial cycling.

In tmf mutants, a single flower with one or more enlarged leaf-like sepals forms in place of the first multi-flowered inflorescence. tmf mutants are also early flowering, and the precocious termination of the primary meristem occurs without the formation of the SYM or SIM, leading to temporary growth arrest of the shoot. tmf mutants maintain development of canonical axillary meristems, which are eventually released from apical dominance and give rise to shoots with regular inflorescences and SYMs, indicating that canonical axillary meristems are largely insensitive to loss of TMF, although the first inflorescence on these side shoots occasionally produces fewer flowers. The tmf single flower phenotype shows approximately 50% penetrance in the original mutant background. By introgressing tmf into our standard ‘M82’ genotype over three generations, we isolated lines with varying levels of penetrance, including a fully penetrant line, suggesting that unknown modifiers underlie the variable penetrance. Importantly, non-penetrant individuals still flower earlier than wild type, though later than their penetrant siblings. Thus, tmf represents the only known tomato mutant with an inflorescence made of a single flower, mimicking Solanaceae species with simple inflorescences.

### Example 2

### tmf Encodes a Small Nuclear Protein

The tmf mutant was originally derived from an interspecific graft between a tomato Break o'Day (BOD) root stock and an eggplant (Solanum melongena) scion, with one branch of the root stock producing a single tmf flower and progeny seed (Lukyanenko, A. N., E. P. Ochova, and M. Egeyan, A mutant with a single flower terminating the main stem. TGC Report, 1973. 23: p. 24). The tmf mutant (LA2462) as well as Break o′ Day (LA1499) were obtained from the Tomato Genetics Research Center (TGRC) U C Davis, Davis, Calif. tmf was crossed to M82 and backcrossed two additional times to generate a BC3 population. The BC3 F2 individuals showing the tmf morphology were selfed, and their progeny showed varying penetrance ranging from 38% to 100%.

To map the tmf mutation, two F2 mapping populations were generated: one with Solanum pennellii (7 mutants identified out of −500 F2 plants) and a larger population with S. pimpinellifolium (329 mutants identified out of −3000 F2 plants). We generated markers using the genomic sequence of the wild species S. pimpinellifolium in addition to markers publicly available from the Sol Genomics Network.

We mapped tmf to a 47 kb region on chromosome 9, and of the five genes in the mapping interval (FIG. 2A), Solyc09g090180, annotated as a small nuclear protein of unknown function, was expressed in WT apices, but not in mutants (FIG. 6A). We found that an 827 bp fragment containing the complete 627 bp single exon coding region of Solyc09g090180 could not be amplified from the mutant (FIG. 6B), and we detected a structural change in this locus by Southern blot (FIG. 6C). Further PCR revealed that only the 3′ end of Solyc09g090180 could not be amplified due to insertion of a Rider Ty1-copia-like retrotransposon 27 by downstream of the stop codon (FIG. 6D; Cheng, X., et al., A New Family of Ty1-copia-Like Retrotransposons Originated in the Tomato Genome by a Recent Horizontal Transfer Event. Genetics, 2009. 181(4): p. 1183-1193; Xiao, H., et al., A retrotransposon-mediated gene duplication underlies morphological variation of tomato fruit. Science, 2008. 319(5869): p. 1527-30). To confirm that Solyc09g090180 encodes TMF, we isolated a second allele (tmf-2) by TILLING an ethane methyl sulfonate (EMS) mutagenized population (Menda, N., et al., In silico screening of a saturated mutation library of tomato. Plant J, 2004. 38(5): p. 861-72; Henikoff, S., B. J. Till, and L. Comai, TILLING. Traditional Mutagenesis Meets Functional Genomics. Plant Physiology, 2004. 135(2): p. 630-636). The tmf-2 lesion is a missense mutation that converts a highly conserved threonine to an isoleucine (FIGS. 7A and 7B). tmf-2 fails to complement tmf, and, like the classical allele, produces a single-flowered primary inflorescence, fails to initiate sympodial growth, exhibits incomplete penetrance (9%), and flowers early in both penetrant (4.3+/−0.52 leaves) and non-penetrant (6.1+/−0.50 leaves) individuals compared to WT (8.4+/−0.77 leaves).

TMF encodes a member of the ALOG (Arabidopsis Light Sensitive Hypocotyl 1, Oryza G1) gene family of nuclear localized proteins containing a single strongly conserved central domain of unknown function (DUF640; Bateman, A., et al., The Pfam protein families database. Nucleic Acids Research, 2002. 30(1): p. 276-280) with little other sequence homology (Zhao, L., et al., Overexpression of LSH1, a member of an uncharacterised gene family, causes enhanced light regulation of seedling development. The Plant Journal, 2004. 37(5): p. 694-706; Yoshida, A., et al., The homeotic gene long sterile lemma (G1) specifies sterile lemma identity in the rice spikelet. Proceedings of the National Academy of Sciences of the United States of America, 2009. 106(47): p. 20103-8). The tomato genome encodes 11 additional ALOG genes, similar to the 10 member Arabidopsis (Zhao, L., et al., Overexpression of LSH1, a member of an uncharacterised gene family, causes enhanced light regulation of seedling development. The Plant Journal, 2004. 37(5): p. 694-706) and rice (Yoshida, A., et al., The homeotic gene long sterile lemma (G1) specifies sterile lemma identity in the rice spikelet. Proceedings of the National Academy of Sciences of the United States of America, 2009. 106(47): p. 20103-8) families (FIG. 8). The ALOG family is conserved throughout most of the plant kingdom, including the lycophyte Selaginella moellendorffii and the moss Physcomitrella patens, but not the algae Chlamydomonas reinhardtii and Volvox carteri. Only a few ALOG genes have known developmental roles: LSH1 functions in light regulation of Arabidopsis seedling development (Zhao, L., et al., Overexpression of LSH1, a member of an uncharacterised gene family, causes enhanced light regulation of seedling development. The Plant Journal, 2004. 37(5): p. 694-706), a member from a grass-specific Glade of the ALOG family specifies spikelet organs in rice (Yoshida, A., et al., The homeotic gene long sterile lemma (G1) specifies sterile lemma identity in the rice spikelet. Proceedings of the National Academy of Sciences of the United States of America, 2009. 106(47): p. 20103-8), and LSH4 and its redundant homolog LSH3 suppress lateral organ differentiation in Arabidopsis (Takeda, S., et al., CUP-SHAPED COTYLEDONI transcription factor activates the expression of LSH4 and LSH3, two members of the ALOG gene family, in shoot organ boundary cells. The Plant Journal, 2011. 66(6): p. 1066-1077; Cho, E. and P. C. Zambryski, ORGAN BOUNDARY1 defines a gene expressed at the junction between the shoot apical meristem and lateral organs. Proceedings of the National Academy of Sciences, 2011. 108(5): p. 2154-2159).

### Example 3

### TMF Expression Promotes a Vegetative Meristem State and is Reduced at the Transition to Flowering

Semi-quantitative RT-PCR on RNA isolated from a panel of tissues revealed that TMF is expressed solely in shoot apices (FIG. 2B), which include the meristem and young leaf primordia. Our recently established tomato meristem maturation atlas (Park, S. J., et al., Rate of meristem maturation determines inflorescence architecture in tomato. Proceedings of the National Academy of Sciences, 2012. 109(2): p. 639-644) indicated that TMF is expressed in the vegetative stages of the PSM and decreases slightly during the reproductive transition meristem (TM) stage (FIG. 2C). TMF expression then drops low in the flower meristem (FM) and is similarly low in the SIM, but expression in the vegetative SYM is comparable to the primary vegetative meristem. We substantiated these expression dynamics using in situ hybridization, which further revealed that TMF is expressed at the periphery of vegetative meristems, extending into initiating vasculature cells (FIG. 2D-2G).

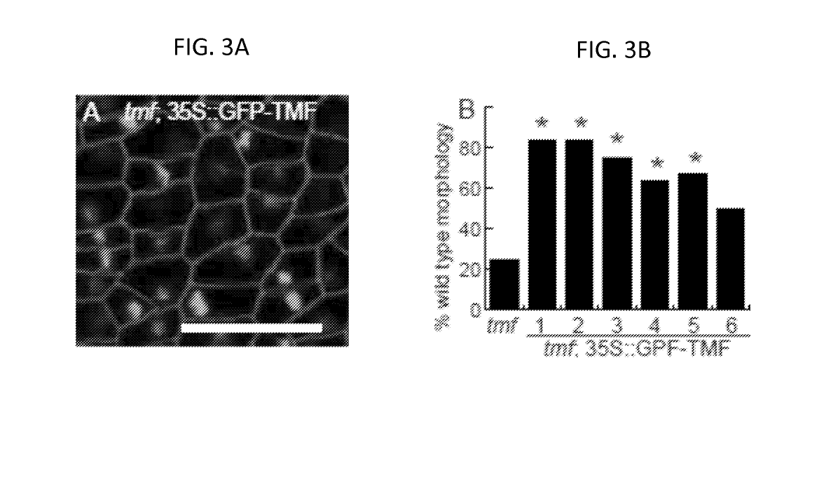

The high expression of TMF during vegetative meristem development and its sudden down-regulation in reproductive meristems suggested a role in maintaining a vegetative meristem state. To explore this idea, we constitutively expressed an N-terminal Green Fluorescent Protein (GFP) fused to the TMF coding sequence under the control of the 35S promoter (35S::GFP-TMF) (FIG. 3A-3B).

The single exon TMF coding region was amplified from genomic DNA using the primers in Table 1. The resulting fragment was topo cloned into pENTR™/D-TOPO® (INVITROGEN™) to generate an entry clone. The entry clone was confirmed by sequencing and subsequently recombined into pMDC4 (Curtis, M. D. and U. Grossniklaus, A Gateway Cloning Vector Set for High-Throughput Functional Analysis of Genes in Planta. Plant Physiology, 2003. 133(2): p. 462-469) to generate the 35S::GFP-TMF construct. The construct was transformed into both Break o′ Day and tmf at the plant transformation facility at the Boyce Thompson Institute using standard methods (Van Eck, J., D. Kirk, and A. Walmsley, Tomato (Lycopersicum esculentum). Methods in Molecular Biology, 2006. 343: p. 459-479), and more than ten transformants were recovered for each genotype. The self-fertilized T2s were scored for flowering time and inflorescence architecture defects (n=12 for each line). Rescue of the tmf phenotype was determined by X² test using the penetrance observed for the non-transformed controls lines (75% giving the tmf phenotype) as the expected values for the tmf vs. wild type phenotype. Overexpression was scored in the primary inflorescence for each incidence of morphological defects (branching, leaves, and reversions to vegetative growth) was quantified and statistical significance was determined by t-test comparing the number of abnormalities between each line to wild type controls.

The GFP-TMF fusion exhibited nuclear localization in agreement with observations for other ALOG family members, and, importantly, rescued all tmf phenotypes, including flowering time (8.0+/−1.1 leaves in transgenics vs. 5.1+/−0.8 leaves in tmf controls, t-test p-value<0.05) and sympodial growth (FIG. 3A-3B). We also noted that inflorescences produced ectopic leaves, reverted to vegetative shoots, and exhibited increased branching at a low but consistent frequency, and the same was true for 35S::GFP-TMF transformed into WT BOD plants. These gain-of-function phenotypes indicated that forcing TMF activity in the FM and SIM can promote a partial vegetative state that directs indeterminacy in reproductive meristems.

### Example 4

### TMF Blocks Activation of a Subset of Flowering Transition and Floral Meristem Identity Genes

To begin investigating how TMF promotes a vegetative state, we performed Illumina mRNA-seq on microdissected meristems at eight days post germination (4^{th} leaf initiated) on tmf mutants and matched WT controls (Table 5).

Differentially expressed genes in vegetative meristems between tmf mutants and Break of the Day were identified. Only those genes showing greater than two fold change and P value≦0.05 were selected.

TABLE 5
Read Number and Mapping Rate for mRNA Sequencing Libraries

| Geno- | | | | Multiple | | |
|---|---|---|---|---|---|---|
| type | R* | Total Reads | Mapped Reads | Mapping | Mapping Rate | Multi Rate |
| tmf | 1 | 29,344,526 | 16,813,393 | 703,035 | 0.572965227 | 0.023957961 |
| tmf | 2 | 39,558,459 | 19,674,165 | 953,296 | 0.49734407 | 0.024098411 |
| BOD | 1 | 44,161,216 | 28,310,492 | 1,220,929 | 0.641071387 | 0.027647087 |
| BOD | 2 | 44,205,239 | 22,977,411 | 1,055,138 | 0.519789317 | 0.023869071 |

*Replicate

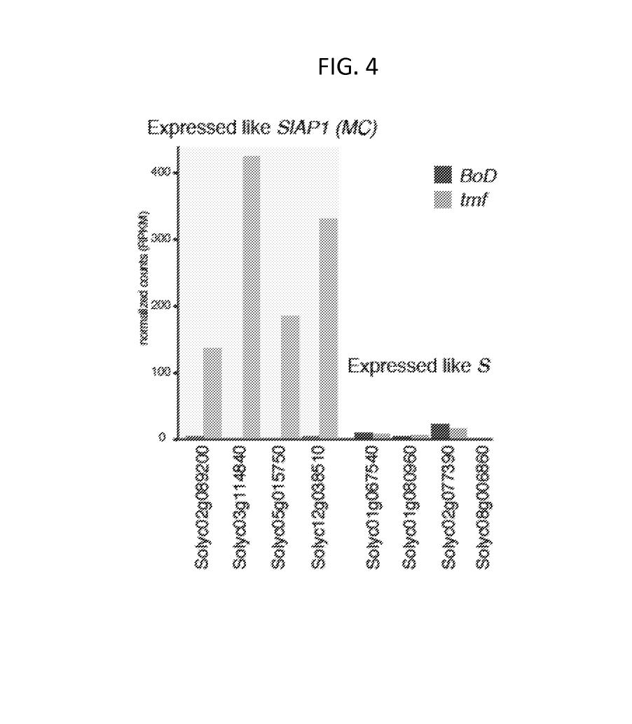

At this early vegetative stage, the apices of tmf and WT PSMs look identical; neither genotype has undergone morphological changes associated with the floral transition. From 17,963 expressed genes, 674 showed greater than two-fold change in tmf compared to WT (False Discovery Rate: FDR, two-fold change and P≦0.05), and the majority (532) was up-regulated in tmf mutants. Most prominent among the up-regulated genes were tomato homologs of several known floral development factors including MADS-box genes like the closest homolog of Arabidopsis APETALA1 (MC: Solyc05g056620) and four members of the SEPALLATA gene family (Solyc05g015750, Solyc02g089200, Solyc03g114840, Solyc12g038510; FIG. 4; Pelaz, S., et al., B and C floral organ identity functions require SEPALLATA MADS-box genes. Nature, 2000. 405(6783): p. 200-203; Uimari, A., et al., Integration of reproductive meristem fates by a SEPALLATA-like MADS-box gene. Proceedings of the National Academy of Sciences of the United States of America, 2004. 101(44): p. 15817-15822). This sampling of expression changes pointed to a precocious initiation of the floral meristem in tmf mutants. To test this idea further, we took advantage of the high resolution tomato gene expression atlas in which thousands of marker genes showing dynamic, age-dependent expression changes define the gradual maturation of the PSM from a vegetative to a terminal flower state (Park, S. J., et al., Rate of meristem maturation determines inflorescence architecture in tomato. Proceedings of the National Academy of Sciences, 2012. 109(2): p. 639-644). We found that a large proportion of expression changes in tmf is represented by genes that are normally dynamically expressed during WT meristem maturation (40% vs. 11% genome-wide), and of greater note, the majority of upregulated genes were those that gradually increase or peak in expression during the TM and FM stages of WT plants, whereas the down-regulated genes typically peak in expression during WT vegetative stages. Thus, loss of TMF is reflected already in the vegetative phase of meristem growth through early upregulation of a subset of reproductive marker genes and early down-regulation of another subset of vegetative marker genes.

The global expression data supported the notion that TMF promotes a vegetative meristem state by preventing precocious flowering and flower initiation; however, while several flowering transition genes were up-regulated prematurely in tmf mutants, many were not. Most conspicuous among these was S (Solyc02g077390), whose transcript accumulation in WT begins in the late vegetative meristem (LVM) stage, peaks in the TM, and defines a late phase of PSM maturation when sympodial meristems initiate (Lippman, Z. B., et al., The Making of a compound inflorescence in tomato and related nightshades. PLoS Biol, 2008. 6(11): p. e288; Park, S. J., et al., Rate of meristem maturation determines inflorescence architecture in tomato. Proceedings of the National Academy of Sciences, 2012. 109(2): p. 639-644). Additional flowering transition marker genes with expression dynamics like S such as Solyc01g067540, Solyc01g080960 and Solyc08g006860 were also not activated precociously (FIG. 4). In fact, only a small subset of FM-enriched marker genes was differentially expressed in tmf mutants, and likewise, most markers enriched in the early vegetative meristem (EVM) stage also remained unchanged. These observations suggested that the primary meristem in tmf has a mixed identity, reflecting a vegetative meristem onto which only a portion of the floral specification program has been imposed. These data further suggested that aspects of the flowering transition have become uncoupled from floral termination in tmf mutants. Consistent with this, the first sepal in the tmf single flower frequently develops as an enlarged leaf-like organ, suggesting that the early adoption of floral fate in tmf mutants leads to inappropriate incorporation of leaf primordia identity into the first floral whorl organs. Moreover, the terminating flower does not immediately release the uppermost axillary meristems from apical dominance as normal flowers do, and hence these meristems fail to adopt a sympodial fate.

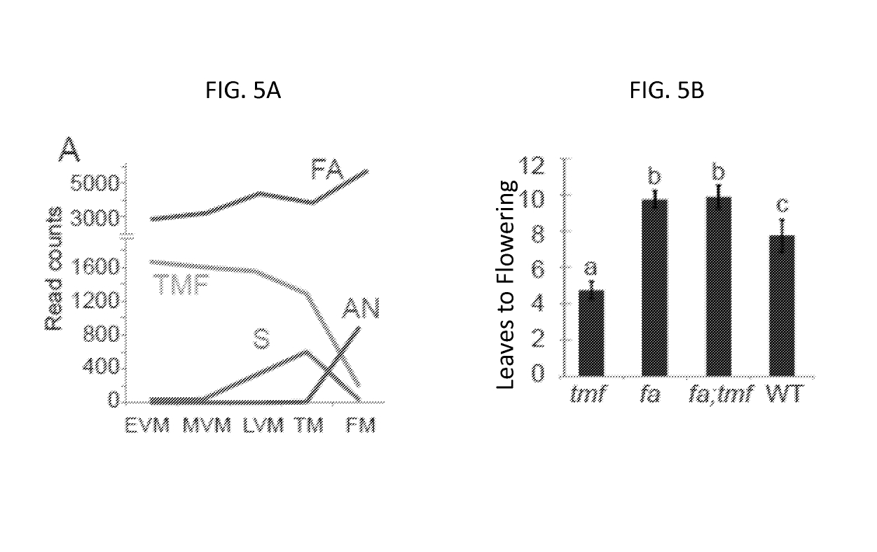

To determine what elements of the floral specification program might be responsible for the precocious adoption of floral fate in tmf mutants, we searched our expression data for misexpression of floral regulators that function in the earliest stages of flower formation. In Arabidopsis, the plant-specific transcription factor LFY is activated in response to combined environmental and endogenous flowering signals and induces flower formation by directly activating floral organ identity genes in all four whorls of the developing flower (William, D. A., et al., Genomic identification of direct target genes of LEAFY. Proceedings of the National Academy of Sciences of the United States of America, 2004. 101(6): p. 1775-1780; Parcy, F., et al., A genetic framework for floral patterning. Nature, 1998. 395(6702): p. 561-566). In tomato, FA (LFY) is gradually up-regulated two-fold during WT PSM maturation (FIG. 5A; Park, S. J., et al., Rate of meristem maturation determines inflorescence architecture in tomato. Proceedings of the National Academy of Sciences, 2012. 109(2): p. 639-644), but we found that FA was already up-regulated 2.2-fold in the vegetative meristem of tmf mutants, suggesting that increased expression of FA in the vegetative phase might be contributing to the tmf phenotypes. Coincidentally, we identified a dominant mutant from a transposon activation tagging population with identical phenotypes to tmf, which we named tmf2-D. Like tmf, tmf2-D mutants showed variable penetrance, were early flowering (tmf2-D/+: 5.8+/−0.85 leaves vs. WT: 7.9+/−0.28 leaves), and produced normal inflorescences from side shoots. We found that the causative insertion was 1.5 kb upstream of the translational start site of FA, revealing that overexpression of FA can recapitulate tmf phenotypes. To test whether FA is required for the tmf syndrome, we took advantage of mutations in FA, which over-proliferate SIMs and produce highly branched leafy inflorescences (Lippman, Z. B., et al., The Making of a compound inflorescence in tomato and related nightshades. PLoS Biol, 2008. 6(11): p. e288; Park, S. J., et al., Rate of meristem maturation determines inflorescence architecture in tomato. Proceedings of the National Academy of Sciences, 2012. 109(2): p. 639-644; Allen, K. D. and I. M. Sussex, Falsiflora and anantha control early stages of floral meristem development in tomato (Lycopersicon esculentum Mill.). Planta, 1996. 200: p. 254-26). We generated tmf;fa double mutants and found that these plants were phenotypically indistinguishable from individual fa mutants in both morphology and flowering time (FIG. 5B). Together, these data indicated that early transcriptional up-regulation of FA can contribute to the tmf mutant phenotypes.

Recent results have shown that LFY activity depends in part on a physical interaction with the F-box protein UFO, which results in a LFY-UFO transcriptional complex that directly activates B-class genes such as AP3 (Chae, E., et al., An Arabidopsis F-box protein acts as a transcriptional co-factor to regulate floral development. Development, 2008. 135(7): p. 1235-1245). Findings in petunia suggest a broader regulatory role for an orthologous LFY-UFO complex (ALF-DOT) in both floral meristem and organ identity of all four whorls (Souer, E., et al., Patterning of Inflorescences and Flowers by the F-Box Protein DOUBLE TOP and the LEAFY Homolog ABERRANT LEAF AND FLOWER of Petunia. The Plant Cell Online, 2008. 20(8): p. 2033-2048). We noted that in addition to the closest tomato homolog of AP3 (SIAP3: Solyc04g081000) showing early up-regulation in tmf vegetative meristems (0.2 read counts in WT vs. 14.6 in tmf), AN (UFO), which is normally activated during early FM development, was precociously activated in tmf vegetative meristems (1.5 read counts in WT vs. 240.6 in tmf) well before S and other late stage PSM markers are normally activated (FIG. 4). Like FA, and consistent with their functional interaction, mutations in AN block floral formation, leading to highly branched inflorescences arrested at the SIM stage (Lippman, Z. B., et al., The Making of a compound inflorescence in tomato and related nightshades. PLoS Biol, 2008. 6(11): p. e288; Allen, K. D. and I. M. Sussex, Falsiflora and anantha control early stages of floral meristem development in tomato (Lycopersicon esculentum Mill.). Planta, 1996. 200: p. 254-264). We tested whether AN was also required for the tmf mutant phenotype by generating tmf; an double mutants, which were indistinguishable from single an mutants. Thus, FA and AN function downstream of TMF, and both genes are required to confer the tmf phenotype. In contrast, we found that S was dispensable, consistent with S transcription remaining unchanged and further supporting the idea that the flowering transition and floral termination are uncoupled in tmf mutants.

Our combined transcriptome and genetic analyses revealed that loss of TMF leads to dramatic ectopic activation of AN in the vegetative meristem, which coincides with upregulation of FA to initiate the AN-FA complex and drive premature acquisition of a floral fate. In petunia, transcriptional activation of DOT/UFO determines when flower formation begins (Souer, E., et al., Patterning of Inflorescences and Flowers by the F-Box Protein DOUBLE TOP and the LEAFY Homolog ABERRANT LEAF AND FLOWER of Petunia. The Plant Cell Online, 2008. 20(8): p. 2033-2048), whereas this role has been assumed by LFY in Arabidopsis (Weigel, D. and O. Nilsson, A developmental switch sufficient for flower initiation in diverse plants. Nature, 1995. 377(6549): p. 495-500), and it has been hypothesized these differences trace back to evolutionary divergence in expression dynamics (Souer, E., et al., Patterning of Inflorescences and Flowers by the F-Box Protein DOUBLE TOP and the LEAFY Homolog ABERRANT LEAF AND FLOWER of Petunia. The Plant Cell Online, 2008. 20(8): p. 2033-2048). We tested the significance of the timing of AN activation in promoting early flowering and inflorescence architectures by trans-activating AN precociously at an early developmental stage of the vegetative meristem using the promoter of the Arabidopsis FILAMENTOUS FOWER (FIL) gene, which is expressed in young vasculature, leaf primordia, and at the periphery of vegetative meristems (Lifschitz, E., et al., The tomato FT ortholog triggers systemic signals that regulate growth and flowering and substitute for diverse environmental stimuli. Proc Natl Acad Sci USA, 2006. 103(16): p. 6398-403). In all cases, pFIL>>AN plants produced a single flower with no sympodial growth after developing only three simple leaves. We next trans-activated AN later in PSM maturation shortly before its normal FM activation using the promoter of the S gene, which peaks in the TM stage (Lippman, Z. B., et al., The Making of a compound inflorescence in tomato and related nightshades. PLoS Biol, 2008. 6(11): p. e288; Park, S. J., et al., Rate of meristem maturation determines inflorescence architecture in tomato. Proceedings of the National Academy of Sciences, 2012. 109(2): p. 639-644). In our strongest lines, pS>>AN plants produced a single flower primary inflorescence like tmf mutants. Remarkably, weaker S promoter lines driving AN maintained stereotypical sympodial vegetative growth with sequential inflorescences having only a single flower, recreating the growth pattern of other Solanaceae species such as N. benthamiana. Thus, altering the timing of AN activation during PSM maturation can quantitatively modulate both the flowering transition and inflorescence architecture.

### Example 5

### TMF Promoter

TMF is highly expressed in tomato during vegetative meristem development and it is suddenly down-regulated in reproductive meristems. For example, TMF is highly expressed in the EVM stage (8 days post-germination). After the floral transition, TMF is expressed again in the SYM, which has a short vegetative phase before transitioning to the next inflorescence. The nucleotide sequence of the tomato TMF promoter is presented in SEQ ID NO:141. The TMF promoter, from tomato or from other species, can be used to drive expression of heterologous genes in plants. For example, the TMF promoter may be used to drive expression of any of the following: screenable marker genes such as GUS and GFP; selectable marker genes such as PAT, GAT and ALS; developmental genes TMF, FIL, S, AN, WUS and ODP2; and agronomic trait genes.

### Example 6

### Phylogenetic Analysis

Additional members of the ALOG gene family in tomato were identified by BLAST search using the TMF and Arabidopsis LSH family protein sequences as BLASTp queries against the predicted protein sequences of ITAG release 2.3. We identified the Selaginella moellendorffii and Physcomitrella patens ALOG family members using a similar strategy through phytozome v7.0. Multiple sequence alignments were generated using ClustalW (Larkin, M. A., et al., Clustal W and Clustal X version 2.0. Bioinformatics, 2007. 23(21): p. 2947-2948). Following sequence alignment, a maximum parsimony phylogenetic tree was calculated with 100 replicate bootstrap values by PHYLIP protpars via the Mobyle portal (Néron, B., et al., Mobyle: a new full web bioinformatics framework. Bioinformatics, 2009. 25(22): p. 3005-3011). At4g19500 was used as the outgroup since it is the most similar Arabidopsis protein that falls outside of the ALOG family.

FIG. 8 shows the phylogenetic tree of the ALOG gene family. The Arabidopsis ALOG family members LSH3 and LSH4 are indicated in parentheses, and are the most similar to TMF.

### Example 7

### Comparison of TMF Homologs from Various Species

FIG. 9A-9B show a multiple alignment of the amino acid sequences of the TMF polypeptides of SEQ ID NOs:46, 48, 62, 63, 65, 70, 75, 81, 88, 90, 91, 93, 100, 102, 109, 111, 116, 123 and 124.

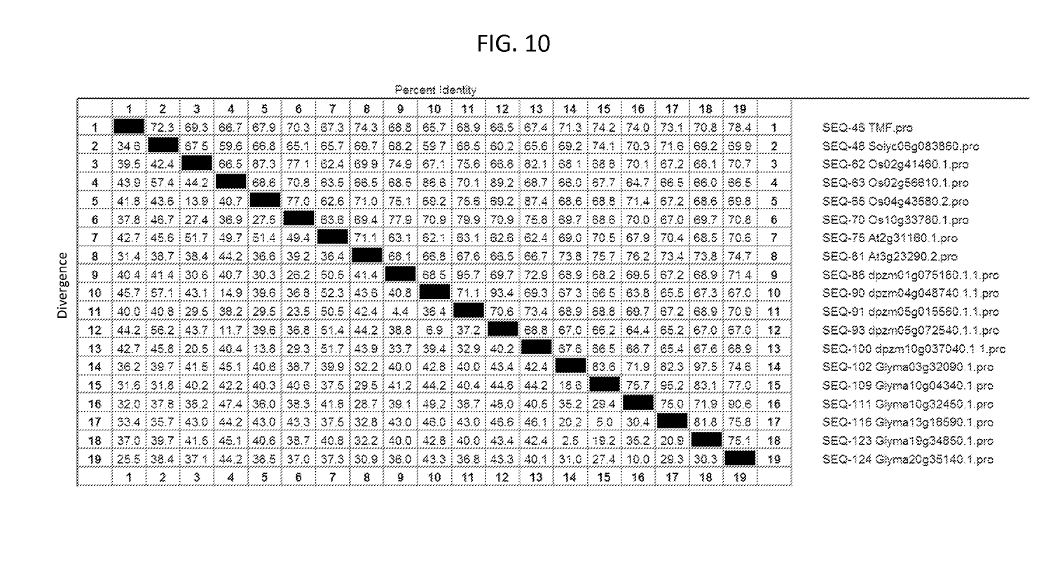

FIG. 10 shows the percent sequence identity and the divergence values for each pair of amino acids sequences of TMF polypeptides displayed in FIGS. 11A-11B.

Sequence alignments and percent identity calculations were performed using the Megalign® program of the LASERGENE® bioinformatics computing suite (DNASTAR® Inc., Madison, Wis.). Multiple alignment of the sequences was performed using the Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB.

### Example 8

### Preparation of a Plant Expression Vector Containing a Homolog to the TMF Polypeptide Gene

Sequences homologous to the TMF polypeptide can be identified using sequence comparison algorithms such as BLAST (Basic Local Alignment Search Tool; Altschul et al., J. Mol. Biol. 215:403-410 (1993); see also the explanation of the BLAST algorithm on the world wide web site for the National Center for Biotechnology Information at the National Library of Medicine of the National Institutes of Health). Sequences encoding homologous TMF polypeptides can be PCR-amplified by any of the following methods.

Method 1 (RNA-based): If the 5′ and 3′ sequence information for the protein-coding region, or the 5′ and 3′ UTR, of a gene encoding a TMF polypeptide homolog is available, gene-specific primers can be designed as outlined in Example 5. RT-PCR can be used with plant RNA to obtain a nucleic acid fragment containing the protein-coding region flanked by attB1 (SEQ ID NO:137) and attB2 (SEQ ID NO:138) sequences. The primer may contain a consensus Kozak sequence (CAACA) upstream of the start codon.

Method 2 (DNA-based): Alternatively, if a cDNA clone is available for a gene encoding a TMF polypeptide homolog, the entire cDNA insert (containing 5′ and 3′ non-coding regions) can be PCR amplified. Forward and reverse primers can be designed that contain either the attB1 sequence and vector-specific sequence that precedes the cDNA insert or the attB2 sequence and vector-specific sequence that follows the cDNA insert, respectively. For a cDNA insert cloned into the vector pBulescript SK+, the forward primer VC062 (SEQ ID NO:139) and the reverse primer VC063 (SEQ ID NO:140) can be used.

Method 3 (genomic DNA): Genomic sequences can be obtained using long range genomic PCR capture. Primers can be designed based on the sequence of the genomic locus and the resulting PCR product can be sequenced. The sequence can be analyzed using the FGENESH (Salamov, A. and Solovyev, V. (2000) Genome Res., 10: 516-522) program, and optionally, can be aligned with homologous sequences from other species to assist in identification of putative introns.

The above methods can be modified according to procedures known by one skilled in the art. For example, the primers of Method 1 may contain restriction sites instead of attB1 and attB2 sites, for subsequent cloning of the PCR product into a vector containing attB1 and attB2 sites. Additionally, Method 2 can involve amplification from a cDNA clone, a lambda clone, a BAC clone or genomic DNA.

A PCR product obtained by either method above can be combined with the GATEWAY® donor vector, such as pDONR™/Zeo (INVITROGEN™) or pDONR™221 (INVITROGEN™), using a BP Recombination Reaction. This process removes the bacteria lethal ccdB gene, as well as the chloramphenicol resistance gene (CAM) from pDONR™221 and directionally clones the PCR product with flanking attB1 and attB2 sites to create an entry clone. Using the INVITROGEN™ GATEWAY® CLONASE™ technology, the sequence encoding the homologous TMF polypeptide from the entry clone can then be transferred to a suitable destination vector to obtain a plant expression vector for use with Arabidopsis, soybean, rice and corn, for example.

Alternatively a MultiSite GATEWAY® LR recombination reaction between multiple entry clones and a suitable destination vector can be performed to create an expression vector.

### Example 9

### Preparation of Soybean Expression Vectors and Transformation of Soybean with TMF Polypeptide Genes

Soybean plants can be transformed to overexpress a TMF polypeptide gene or the corresponding homologs from various species in order to examine the resulting phenotype.

A GATEWAY® entry clone can be used to directionally clone each gene into an appropriate vector such that expression of the gene is under control of the SCP1 promoter (International Publication No. 03/033651).

Soybean embryos may then be transformed with the expression vector comprising sequences encoding the instant polypeptides. Techniques for soybean transformation and regeneration have been described in International Patent Publication WO 2009/006276, the contents of which are herein incorporated by reference.

Soybean plants transformed with an expression vector can then be assayed under field-based studies to study yield enhancement and/or stability under stressed and non-stressed conditions.

### Example 10

### Transformation of Maize with TMF Polypeptide Genes Using Particle Bombardment

Maize plants can be transformed to overexpress a TMF polypeptide gene or the corresponding homologs from various species in order to examine the resulting phenotype.

A GATEWAY® entry clone can be used to directionally clone each gene into a maize transformation vector. Expression of the gene in the maize transformation vector can be under control of a constitutive promoter such as the maize ubiquitin promoter (Christensen et al., (1989) Plant Mol. Biol. 12:619-632 and Christensen et al., (1992) Plant Mol. Biol. 18:675-689)

The recombinant DNA construct described above can then be introduced into corn cells by particle bombardment. Techniques for corn transformation by particle bombardment have been described in International Patent Publication WO 2009/006276, the contents of which are herein incorporated by reference.

Maize plants transformed with an expression vector can then be assayed under field-based studies to study yield enhancement and/or stability under stressed and non-stressed conditions.

### Example 11

### Electroporation of Agrobacterium tumefaciens LBA4404

Electroporation competent cells (40 μL), such as Agrobacterium tumefaciens LBA4404 containing PHP10523, are thawed on ice (20-30 min). PHP10523 contains VIR genes for T-DNA transfer, an Agrobacterium low copy number plasmid origin of replication, a tetracycline resistance gene, and a Cos site for in vivo DNA bimolecular recombination. Meanwhile the electroporation cuvette is chilled on ice. The electroporator settings are adjusted to 2.1 kV. A DNA aliquot (0.5 μL parental DNA at a concentration of 0.2 μg-1.0 μg in low salt buffer or twice distilled H₂O) is mixed with the thawed Agrobacterium tumefaciens LBA4404 cells while still on ice. The mixture is transferred to the bottom of electroporation cuvette and kept at rest on ice for 1-2 min. The cells are electroporated (Eppendorf electroporator 2510) by pushing the “pulse” button twice (ideally achieving a 4.0 millisecond pulse). Subsequently, 0.5 mL of room temperature 2×YT medium (or SOC medium) are added to the cuvette and transferred to a 15 mL snap-cap tube (e.g., FALCON™ tube). The cells are incubated at 28-30° C., 200-250 rpm for 3 h.

Aliquots of 250 μL are spread onto plates containing YM medium and 50 μg/mL spectinomycin and incubated three days at 28-30° C. To increase the number of transformants one of two optional steps can be performed:

Option 1: Overlay plates with 30 μL of 15 mg/mL rifampicin. LBA4404 has a chromosomal resistance gene for rifampicin. This additional selection eliminates some contaminating colonies observed when using poorer preparations of LBA4404 competent cells.

Option 2: Perform two replicates of the electroporation to compensate for poorer electrocompetent cells.

Identification of Transformants:

Four independent colonies are picked and streaked on plates containing AB minimal medium and 50 μg/mL spectinomycin for isolation of single colonies. The plates are incubated at 28° C. for two to three days. A single colony for each putative co-integrate is picked and inoculated with 4 mL of 10 g/L bactopeptone, 10 g/L yeast extract, 5 g/L sodium chloride and 50 mg/L spectinomycin. The mixture is incubated for 24 h at 28° C. with shaking. Plasmid DNA from 4 mL of culture is isolated using Qiagen® Miniprep and an optional Buffer PB wash. The DNA is eluted in 30 μL. Aliquots of 2 μL are used to electroporate 20 μL of DH10b+20 μL of twice distilled H₂O as per above. Optionally a 15 μL aliquot can be used to transform 75-100 μL of INVITROGEN™ Library Efficiency DH5α. The cells are spread on plates containing LB medium and 50 μg/mL spectinomycin and incubated at 37° C. overnight.

Three to four independent colonies are picked for each putative co-integrate and inoculated 4 mL of 2×YT medium (10 g/L bactopeptone, 10 g/L yeast extract, 5 g/L sodium chloride) with 50 μg/mL spectinomycin. The cells are incubated at 37° C. overnight with shaking. Next, isolate the plasmid DNA from 4 mL of culture using QIAprep® Miniprep with optional Buffer PB wash (elute in 50 μL). Use 8 μL for digestion with SalI (using parental DNA and PHP10523 as controls). Three more digestions using restriction enzymes BamHI, EcoRI, and HindIII are performed for 4 plasmids that represent 2 putative co-integrates with correct SalI digestion pattern (using parental DNA and PHP10523 as controls). Electronic gels are recommended for comparison.

### Example 12

### Transformation of Maize Using Agrobacterium

Maize plants can be transformed to overexpress a TMF polypeptide gene or the corresponding homologs from various species in order to examine the resulting phenotype.

Agrobacterium-mediated transformation of maize is performed essentially as described by Zhao et al. in Meth. Mol. Biol. 318:315-323 (2006) (see also Zhao et al., Mol. Breed. 8:323-333 (2001) and U.S. Pat. No. 5,981,840 issued Nov. 9, 1999, incorporated herein by reference). The transformation process involves bacterium innoculation, co-cultivation, resting, selection and plant regeneration.

1. Immature Embryo Preparation:

Immature maize embryos are dissected from caryopses and placed in a 2 mL microtube containing 2 mL PHI-A medium.

2. Agrobacterium Infection and Co-Cultivation of Immature Embryos:

2.1 Infection Step:

PHI-A medium of (1) is removed with 1 mL micropipettor, and 1 mL of Agrobacterium suspension is added. The tube is gently inverted to mix. The mixture is incubated for 5 min at room temperature.

2.2 Co-Culture Step:

The Agrobacterium suspension is removed from the infection step with a 1 mL micropipettor. Using a sterile spatula the embryos are scraped from the tube and transferred to a plate of PHI-B medium in a 100×15 mm Petri dish. The embryos are oriented with the embryonic axis down on the surface of the medium. Plates with the embryos are cultured at 20° C., in darkness, for three days. L-Cysteine can be used in the co-cultivation phase. With the standard binary vector, the co-cultivation medium supplied with 100-400 mg/L L-cysteine is critical for recovering stable transgenic events.

3. Selection of Putative Transgenic Events:

To each plate of PHI-D medium in a 100×15 mm Petri dish, 10 embryos are transferred, maintaining orientation and the dishes are sealed with parafilm. The plates are incubated in darkness at 28° C. Actively growing putative events, as pale yellow embryonic tissue, are expected to be visible in six to eight weeks. Embryos that produce no events may be brown and necrotic, and little friable tissue growth is evident. Putative transgenic embryonic tissue is subcultured to fresh PHI-D plates at two-three week intervals, depending on growth rate. The events are recorded.

4. Regeneration of T0 Plants:

Embryonic tissue propagated on PHI-D medium is subcultured to PHI-E medium (somatic embryo maturation medium), in 100×25 mm Petri dishes and incubated at 28° C., in darkness, until somatic embryos mature, for about ten to eighteen days. Individual, matured somatic embryos with well-defined scutellum and coleoptile are transferred to PHI-F embryo germination medium and incubated at 28° C. in the light (about 80 μE from cool white or equivalent fluorescent lamps). In seven to ten days, regenerated plants, about 10 cm tall, are potted in horticultural mix and hardened-off using standard horticultural methods.

Media for Plant Transformation:
PHI-A: 4 g/L CHU basal salts, 1.0 mL/L 1000× Eriksson's vitamin mix, 0.5 mg/L thiamin HCl, 1.5 mg/L 2,4-D, 0.69 g/L L-proline, 68.5 g/L sucrose, 36 g/L glucose, pH 5.2. Add 100 μM acetosyringone (filter-sterilized).
2. PHI-B: PHI-A without glucose, increase 2,4-D to 2 mg/L, reduce sucrose to 30 g/L and supplemented with 0.85 mg/L silver nitrate (filter-sterilized), 3.0 g/L Gelrite®, 100 μM acetosyringone (filter-sterilized), pH 5.8.
3. PHI-C: PHI-B without Gelrite® and acetosyringonee, reduce 2,4-D to 1.5 mg/L and supplemented with 8.0 g/L agar, 0.5 g/L 2-[N-morpholino]ethane-sulfonic acid (MES) buffer, 100 mg/L carbenicillin (filter-sterilized).
4. PHI-D: PHI-C supplemented with 3 mg/L bialaphos (filter-sterilized).
5. PHI-E: 4.3 g/L of Murashige and Skoog (MS) salts, (Gibco, BRL 11117-074), 0.5 mg/L nicotinic acid, 0.1 mg/L thiamine HCl, 0.5 mg/L pyridoxine HCl, 2.0 mg/L glycine, 0.1 g/L myo-inositol, 0.5 mg/L zeatin (Sigma, Cat. No. Z-0164), 1 mg/L indole acetic acid (IAA), 26.4 μg/L abscisic acid (ABA), 60 g/L sucrose, 3 mg/L bialaphos (filter-sterilized), 100 mg/L carbenicillin (filter-sterilized), 8 g/L agar, pH 5.6.
6. PHI-F: PHI-E without zeatin, IAA, ABA; reduce sucrose to 40 g/L; replacing agar with 1.5 g/L Gelrite®; pH 5.6.

Plants can be regenerated from the transgenic callus by first transferring clusters of tissue to N6 medium supplemented with 0.2 mg per liter of 2,4-D. After two weeks the tissue can be transferred to regeneration medium (Fromm et al., Bio/Technology 8:833-839 (1990)).

Transgenic T0 plants can be regenerated and their phenotype determined. T1 seed can be collected.

Furthermore, a recombinant DNA construct containing a gene of interest can be introduced into an elite maize inbred line either by direct transformation or introgression from a separately transformed line.

Transgenic plants, either inbred or hybrid, can undergo field-based experiments to study yield enhancement and/or stability under stressed and non-stressed conditions.

### Example 13

### Transformation of Gaspe Flint Derived Maize Lines

Maize plants can be transformed to overexpress the TMF polypeptide gene or the corresponding homologs from other species in order to examine the resulting phenotype.

Recipient Plants:

Recipient plant cells can be from a uniform maize line having a short life cycle (“fast cycling”), a reduced size, and high transformation potential. Typical of these plant cells for maize are plant cells from any of the publicly available Gaspe Flint (GBF) line varieties. One possible candidate plant line variety is the F1 hybrid of GBF×QTM (Quick Turnaround Maize, a publicly available form of Gaspe Flint selected for growth under greenhouse conditions) disclosed in Tomes et al. U.S. Patent Application Publication No. 2003/0221212. Transgenic plants obtained from this line are of such a reduced size that they can be grown in four inch pots (¼ the space needed for a normal sized maize plant) and mature in less than 2.5 months. (Traditionally 3.5 months is required to obtain transgenic T0 seed once the transgenic plants are acclimated to the greenhouse.) Another suitable line is a double haploid line of GS3 (a highly transformable line)×Gaspe Flint. Yet another suitable line is a transformable elite inbred line carrying a transgene which causes early flowering, reduced stature, or both.

Transformation Protocol:

Any suitable method may be used to introduce the transgenes into the maize cells, including but not limited to inoculation type procedures using Agrobacterium based vectors. Transformation may be performed on immature embryos of the recipient (target) plant.

Precision Growth and Plant Tracking:

The event population of transgenic (T0) plants resulting from the transformed maize embryos is grown in a controlled greenhouse environment using a modified randomized block design to reduce or eliminate environmental error. A randomized block design is a plant layout in which the experimental plants are divided into groups (e.g., thirty plants per group), referred to as blocks, and each plant is randomly assigned a location with the block.

For a group of thirty plants, twenty-four transformed, experimental plants and six control plants (plants with a set phenotype) (collectively, a “replicate group”) are placed in pots which are arranged in an array (a.k.a. a replicate group or block) on a table located inside a greenhouse. Each plant, control or experimental, is randomly assigned to a location with the block which is mapped to a unique, physical greenhouse location as well as to the replicate group. Multiple replicate groups of thirty plants each may be grown in the same greenhouse in a single experiment. The layout (arrangement) of the replicate groups should be determined to minimize space requirements as well as environmental effects within the greenhouse. Such a layout may be referred to as a compressed greenhouse layout.

An alternative to the addition of a specific control group is to identify those transgenic plants that do not express the gene of interest. A variety of techniques such as RT-PCR can be applied to quantitatively assess the expression level of the introduced gene. T0 plants that do not express the transgene can be compared to those which do.

Each plant in the event population is identified and tracked throughout the evaluation process, and the data gathered from that plant is automatically associated with that plant so that the gathered data can be associated with the transgene carried by the plant. For example, each plant container can have a machine readable label (such as a Universal Product Code (UPC) bar code) which includes information about the plant identity, which in turn is correlated to a greenhouse location so that data obtained from the plant can be automatically associated with that plant.

Alternatively any efficient, machine readable, plant identification system can be used, such as two-dimensional matrix codes or even radio frequency identification tags (RFID) in which the data is received and interpreted by a radio frequency receiver/processor. See U.S. Published Patent Application No. 2004/0122592, incorporated herein by reference.

Phenotypic Analysis Using Three-Dimensional Imaging:

Each greenhouse plant in the T0 event population, including any control plants, is analyzed for agronomic characteristics of interest, and the agronomic data for each plant is recorded or stored in a manner so that it is associated with the identifying data (see above) for that plant. Confirmation of a phenotype (gene effect) can be accomplished in the T1 generation with a similar experimental design to that described above.

The T0 plants are analyzed at the phenotypic level using quantitative, non-destructive imaging technology throughout the plant's entire greenhouse life cycle to assess the traits of interest. A digital imaging analyzer may be used for automatic multi-dimensional analyzing of total plants. The imaging may be done inside the greenhouse. Two camera systems, located at the top and side, and an apparatus to rotate the plant, are used to view and image plants from all sides. Images are acquired from the top, front and side of each plant. All three images together provide sufficient information to evaluate the biomass, size and morphology of each plant.

Due to the change in size of the plants from the time the first leaf appears from the soil to the time the plants are at the end of their development, the early stages of plant development are best documented with a higher magnification from the top. This may be accomplished by using a motorized zoom lens system that is fully controlled by the imaging software.

In a single imaging analysis operation, the following events occur: (1) the plant is conveyed inside the analyzer area, rotated 360 degrees so its machine readable label can be read, and left at rest until its leaves stop moving; (2) the side image is taken and entered into a database; (3) the plant is rotated 90 degrees and again left at rest until its leaves stop moving, and (4) the plant is transported out of the analyzer.

Plants are allowed at least six hours of darkness per twenty four hour period in order to have a normal day/night cycle.

Imaging Instrumentation:

Any suitable imaging instrumentation may be used, including but not limited to light spectrum digital imaging instrumentation commercially available from LemnaTec GmbH of Wurselen, Germany. The images are taken and analyzed with a LemnaTec Scanalyzer HTS LT-0001-2 having a ½″ IT Progressive Scan IEE CCD imaging device. The imaging cameras may be equipped with a motor zoom, motor aperture and motor focus. All camera settings may be made using LemnaTec software. For example, the instrumental variance of the imaging analyzer is less than about 5% for major components and less than about 10% for minor components.

Software:

The imaging analysis system comprises a LemnaTec HTS Bonit software program for color and architecture analysis and a server database for storing data from about 500,000 analyses, including the analysis dates. The original images and the analyzed images are stored together to allow the user to do as much reanalyzing as desired. The database can be connected to the imaging hardware for automatic data collection and storage. A variety of commercially available software systems (e.g. Matlab, others) can be used for quantitative interpretation of the imaging data, and any of these software systems can be applied to the image data set.

Conveyor System:

A conveyor system with a plant rotating device may be used to transport the plants to the imaging area and rotate them during imaging. For example, up to four plants, each with a maximum height of 1.5 m, are loaded onto cars that travel over the circulating conveyor system and through the imaging measurement area. In this case the total footprint of the unit (imaging analyzer and conveyor loop) is about 5 m×5 m.

The conveyor system can be enlarged to accommodate more plants at a time. The plants are transported along the conveyor loop to the imaging area and are analyzed for up to 50 seconds per plant. Three views of the plant are taken. The conveyor system, as well as the imaging equipment, should be capable of being used in greenhouse environmental conditions.

Illumination:

Any suitable mode of illumination may be used for the image acquisition. For example, a top light above a black background can be used. Alternatively, a combination of top- and backlight using a white background can be used. The illuminated area should be housed to ensure constant illumination conditions. The housing should be longer than the measurement area so that constant light conditions prevail without requiring the opening and closing or doors. Alternatively, the illumination can be varied to cause excitation of either transgene (e.g., green fluorescent protein (GFP), red fluorescent protein (RFP)) or endogenous (e.g. Chlorophyll) fluorophores.

Biomass Estimation Based on Three-Dimensional Imaging:

For best estimation of biomass the plant images should be taken from at least three axes, for example, the top and two side (sides 1 and 2) views. These images are then analyzed to separate the plant from the background, pot and pollen control bag (if applicable). The volume of the plant can be estimated by the calculation:

Volume(voxels)=\sqrt{TopArea(pixels)}×\sqrt{Side1Area(pixels)}×\sqrt{Side2Area(pixels)}

In the equation above the units of volume and area are “arbitrary units”. Arbitrary units are entirely sufficient to detect gene effects on plant size and growth in this system because what is desired is to detect differences (both positive-larger and negative-smaller) from the experimental mean, or control mean. The arbitrary units of size (e.g. area) may be trivially converted to physical measurements by the addition of a physical reference to the imaging process. For instance, a physical reference of known area can be included in both top and side imaging processes. Based on the area of these physical references a conversion factor can be determined to allow conversion from pixels to a unit of area such as square centimeters (cm²). The physical reference may or may not be an independent sample. For instance, the pot, with a known diameter and height, could serve as an adequate physical reference.

Color Classification:

The imaging technology may also be used to determine plant color and to assign plant colors to various color classes. The assignment of image colors to color classes is an inherent feature of the LemnaTec software. With other image analysis software systems color classification may be determined by a variety of computational approaches.

For the determination of plant size and growth parameters, a useful classification scheme is to define a simple color scheme including two or three shades of green and, in addition, a color class for chlorosis, necrosis and bleaching, should these conditions occur. A background color class which includes non plant colors in the image (for example pot and soil colors) is also used and these pixels are specifically excluded from the determination of size. The plants are analyzed under controlled constant illumination so that any change within one plant over time, or between plants or different batches of plants (e.g. seasonal differences) can be quantified.

In addition to its usefulness in determining plant size growth, color classification can be used to assess other yield component traits. For these other yield component traits additional color classification schemes may be used. For instance, the trait known as “staygreen”, which has been associated with improvements in yield, may be assessed by a color classification that separates shades of green from shades of yellow and brown (which are indicative of senescing tissues). By applying this color classification to images taken toward the end of the T0 or T1 plants' life cycle, plants that have increased amounts of green colors relative to yellow and brown colors (expressed, for instance, as Green/Yellow Ratio) may be identified. Plants with a significant difference in this Green/Yellow ratio can be identified as carrying transgenes which impact this important agronomic trait.

The skilled plant biologist will recognize that other plant colors arise which can indicate plant health or stress response (for instance anthocyanins), and that other color classification schemes can provide further measures of gene action in traits related to these responses.

Plant Architecture Analysis:

Transgenes which modify plant architecture parameters may also be identified using the present invention, including such parameters as maximum height and width, internodal distances, angle between leaves and stem, number of leaves starting at nodes and leaf length. The LemnaTec system software may be used to determine plant architecture as follows. The plant is reduced to its main geometric architecture in a first imaging step and then, based on this image, parameterized identification of the different architecture parameters can be performed. Transgenes that modify any of these architecture parameters either singly or in combination can be identified by applying the statistical approaches previously described.

Pollen Shed Date:

Pollen shed date is an important parameter to be analyzed in a transformed plant, and may be determined by the first appearance on the plant of an active male flower. To find the male flower object, the upper end of the stem is classified by color to detect yellow or violet anthers. This color classification analysis is then used to define an active flower, which in turn can be used to calculate pollen shed date.

Alternatively, pollen shed date and other easily visually detected plant attributes (e.g. pollination date, first silk date) can be recorded by the personnel responsible for performing plant care. To maximize data integrity and process efficiency this data is tracked by utilizing the same barcodes utilized by the LemnaTec light spectrum digital analyzing device. A computer with a barcode reader, a palm device, or a notebook PC may be used for ease of data capture recording time of observation, plant identifier, and the operator who captured the data.

Orientation of the Plants:

Mature maize plants grown at densities approximating commercial planting often have a planar architecture. That is, the plant has a clearly discernable broad side, and a narrow side. The image of the plant from the broadside is determined. To each plant a well defined basic orientation is assigned to obtain the maximum difference between the broadside and edgewise images. The top image is used to determine the main axis of the plant, and an additional rotating device is used to turn the plant to the appropriate orientation prior to starting the main image acquisition.

### Example 14

### Transformation of Rice with TMF Polypeptide Genes

Rice plants can be transformed to overexpress a TMF polypeptide gene or the corresponding homologs from various species in order to examine the resulting phenotype.

Immature embryos, e.g., of proprietary Indica strain 851G, can be transformed using the methods disclosed in International Application Publication No. WO/1995/06722 and Hiei and Komari (2006) Plant Cell, Tissue and Organ Culture 85:271-283, each of which is herein incorporated by reference in its entirety.

Rice plants transformed with an expression vector can then be assayed under field-based studies to study yield enhancement and/or stability under stressed and non-stressed conditions.

### Example 15

### Expression Using the TMF Promoter

A 3.9-kb DNA fragment (SEQ ID NO:141) containing the promoter of TMF was used to drive expression of ANANTHA, a flowering gene. The expression unit contained the following: the TMF promoter (SEQ ID NO:141); the coding region of ANANTHA (SEQ ID NO:142); and the octopine synthase (OCS) transcription terminator (SEQ ID NO:143). The resulting transgenic plant showed early flowering. Flowering was observed after only two leaves of development, as compared to eight leaves in wild-type tomato plants.

1. A recombinant DNA construct comprising an isolated polynucleotide operably linked to at least one heterologous regulatory sequence, wherein the polynucleotide encodes a polypeptide having an amino acid sequence of at least 80% sequence identity, based on the Clustal W method of alignment, when compared to SEQ ID NO:46, 48, 62, 63, 65, 70, 75, 81, 88, 90, 91, 93, 100, 102, 109, 111, 116, 123 or 124, wherein expression of the polynucleotide in a transgenic plant can increase at least one agronomic characteristic selected from the group consisting of: leaf number, inflorescence number, branching within the inflorescence, flower number, fruit number, seed number, biomass and yield, when compared to a control plant not comprising the recombinant DNA construct.

2. The recombinant DNA construct of claim 1, wherein expression of the polynucleotide in a tomato line having the tmf mutant genotype is capable of partially or fully restoring the wild-type phenotype.

3. A method of producing a transgenic plant with an increase of an agronomic characteristic, the method comprising:
(a) introducing into a regenerable plant cell the recombinant DNA construct of claim 1;
(b) regenerating a transgenic plant from the regenerable plant cell after step (a), wherein the transgenic plant comprises in its genome the recombinant DNA construct; and
(c) selecting a transgenic plant of (b), wherein the transgenic plant comprises the recombinant DNA construct, wherein the polynucleotide is expressed, and wherein the plant exhibits an increase of at least one agronomic characteristic selected from the group consisting of: leaf number, inflorescence number, branching within the inflorescence, flower number, fruit number, seed number, biomass and yield, when compared to a control plant not comprising the recombinant DNA construct.

4. (canceled)

5. A plant comprising in its genome the recombinant DNA construct of claim 1, wherein the plant exhibits an increase of at least one agronomic characteristic selected from the group consisting of: leaf number, inflorescence number, branching within the inflorescence, flower number, fruit number, seed number, biomass and yield, when compared to a control plant not comprising the recombinant DNA construct.

6. The plant of claim 5, wherein said plant is selected from the group consisting of: Arabidopsis, tomato, maize, soybean, sunflower, sorghum, canola, wheat, alfalfa, cotton, rice, barley, millet, sugar cane and switchgrass.

7. A seed of the plant of claim 5, wherein said seed comprises in its genome the recombinant DNA construct of claim 1, and wherein a plant produced from the seed exhibits an increase of at least one agronomic characteristic selected from the group consisting of: leaf number, inflorescence number, branching within the inflorescence, flower number, fruit number, seed number, biomass and yield, when compared to a control plant not comprising the recombinant DNA construct.

8. A suppression DNA construct comprising an isolated polynucleotide operably linked, in sense or antisense orientation, to a heterologous promoter functional in a plant, wherein the polynucleotide comprises:
(a) a first nucleotide sequence encoding a polypeptide having an amino acid sequence of SEQ ID NO:46, 48, 62, 63, 65, 70, 75, 81, 88, 90, 91, 93, 100, 102, 109, 111, 116, 123 or 124;
(b) a second nucleotide sequence having at least 90% sequence identity, when compared to the first nucleotide sequence;
(c) a third nucleotide sequence of at least 100 contiguous nucleotides of the first nucleotide sequence;
(d) a fourth nucleotide sequence that can hybridize under stringent conditions with the first nucleotide sequence; or
(e) a modified plant miRNA precursor, wherein the precursor has been modified to replace the miRNA encoding region with a sequence designed to produce a miRNA directed to the first nucleotide sequence,
wherein the suppression DNA construct induces an earlier flowering time in a transgenic plant, when compared to a control plant not comprising the suppression DNA construct

9. A method of producing a transgenic plant with an earlier flowering time, the method comprising:
(a) introducing into a regenerable plant cell the suppression DNA construct of claim 8;
(b) regenerating a transgenic plant from the regenerable plant cell after step (a), wherein the transgenic plant comprises in its genome the suppression DNA construct; and
(c) selecting a transgenic plant of (b), wherein the transgenic plant comprises the suppression DNA construct and exhibits an earlier flowering time, when compared to a control plant not comprising the suppression DNA construct.

10. (canceled)

11. A plant comprising in its genome the suppression DNA construct of claim 8, wherein the plant exhibits an earlier flowering time, when compared to a control plant not comprising the recombinant DNA construct.

12. The plant of claim 11, wherein said plant is selected from the group consisting of: Arabidopsis, tomato, maize, soybean, sunflower, sorghum, canola, wheat, alfalfa, cotton, rice, barley, millet, sugar cane and switchgrass.

13. A seed of the plant of claim 11, wherein said seed comprises in its genome the suppression DNA construct of claim 8, and wherein a plant produced from the seed exhibits an earlier flowering time, when compared to a control plant not comprising the suppression DNA construct.

14. A recombinant DNA construct comprising a heterologous polynucleotide operably linked to a second polynucleotide, wherein the second polynucleotide has a nucleotide sequence selected from the group consisting of:
(a) a first nucleotide sequence comprising SEQ ID NO:141;
(b) a second nucleotide sequence having at least 90% sequence identity, when compared to SEQ ID NO:141;
(c) a third nucleotide sequence of at least 100 contiguous nucleotides of SEQ ID NO:141; or
(d) a fourth nucleotide sequence that can hybridize under stringent conditions with SEQ ID NO:141; and
wherein said second polynucleotide has promoter activity in a plant.

15. A method of expressing a heterologous polynucleotide in a plant, the method comprising:
(a) transforming a regenerable plant cell with the recombinant DNA construct of claim 14;
(b) regenerating a transgenic plant from the regenerable plant cell after step (a), wherein the transgenic plant comprises in its genome the recombinant DNA construct; and
(c) selecting a transgenic plant of step (b), wherein the transgenic plant comprises the recombinant DNA construct and further wherein the heterologous polynucleotide is expressed in the transgenic plant.

16. (canceled)

17. A plant comprising in its genome the recombinant DNA construct of claim 14, wherein the heterologous polynucleotide is expressed in the plant.

18. The plant of claim 17, wherein said plant is selected from the group consisting of: Arabidopsis, tomato, maize, soybean, sunflower, sorghum, canola, wheat, alfalfa, cotton, rice, barley, millet, sugar cane and switchgrass.

19. Seed of the plant of claim 17, wherein said seed comprises in its genome the recombinant DNA construct of claim 14, and wherein the heterologous polynucleotide is expressed in a plant produced from the seed.

20. A method of producing a transgenic plant with an increased seed yield, the method comprising:
(a) introducing into a regenerable plant cell the recombinant DNA construct of claim 1 and the suppression DNA construct of claim 8, wherein the isolated polynucleotide of the recombinant DNA construct is operably linked to a promoter functional in a plant female inflorescence tissue, and wherein the isolated polynucleotide of suppression DNA construct is operably linked, in sense or antisense orientation, to a promoter functional in a plant male inflorescence tissue;
(b) regenerating a transgenic plant from the regenerable plant cell after step (a), wherein the transgenic plant comprises in its genome the suppression DNA construct and the overexpression DNA construct; and
(c) selecting a transgenic plant of (b), wherein the transgenic plant comprises the recombinant DNA construct and the suppression DNA construct and exhibits an increased seed yield, when compared to a control plant not comprising the suppression DNA construct and the overexpression DNA construct.

21. The method of claim 20, wherein said plant is selected from the group consisting of: maize, rice, wheat, sorghum and canola.

22. A method of producing a transgenic plant with an increased seed yield, the method comprising crossing the following:
(a) a first transgenic plant comprising the recombinant DNA construct of claim 1, wherein the isolated polynucleotide is operably linked to a promoter functional in a plant female inflorescence tissue; with
(b) a second transgenic plant comprising the suppression DNA construct of claim 8, wherein the isolated polynucleotide is operably linked, in sense or antisense orientation, to a promoter functional in a plant male inflorescence tissue; and
selecting a transgenic progeny plant of the cross, wherein the transgenic progeny plant comprises the recombinant DNA construct and the suppression DNA construct and exhibits an increased seed yield, when compared to a control plant not comprising the recombinant DNA construct and the suppression DNA construct.

23. The method of claim 22, wherein said plant is selected from the group consisting of: maize, rice, wheat, sorghum and canola.


SEQUENCE LISTING (179 sequences)

SEQ ID NO: 1 - DNA, 27 bp, Artificial sequence
  Solyc09g090180 seqf primer
aaatagtaataatagggaaaaataggg

SEQ ID NO: 2 - DNA, 20 bp, Artificial sequence
  Solyc09g090180 seqr primer
acctctcttctctctctccc

SEQ ID NO: 3 - DNA, 26 bp, Artificial sequence
  Solyc09g090160 f primer
aaataaatacagaggaaaatttttgc

SEQ ID NO: 4 - DNA, 20 bp, Artificial sequence
  Solyc09g090160 r primer
tctgcatatgcgtttactgc

SEQ ID NO: 5 - DNA, 19 bp, Artificial sequence
  Solyc09g090160 seq primer
agaaagcctttcaggttgg

SEQ ID NO: 6 - DNA, 28 bp, Artificial sequence
  Solyc09g090130 f primer
cataaaaaaataaaaaaattcatcaaag

SEQ ID NO: 7 - DNA, 22 bp, Artificial sequence
  Solyc09g090130 seqr primer
aattgtttgaacttttcaaggc

SEQ ID NO: 8 - DNA, 26 bp, Artificial sequence
  Solyc09g090170 f primer
gaaatattccaataataatttggacc

SEQ ID NO: 9 - DNA, 23 bp, Artificial sequence
  Solyc09g090170 r primer
ttcttctcaaaccatttaattcc

SEQ ID NO: 10 - DNA, 21 bp, Artificial sequence
  SSR112f primer
ggaacacaaccaagaagtgga

SEQ ID NO: 11 - DNA, 20 bp, Artificial sequence
  SSR112r primer
tatcggcttagggttgttgg

SEQ ID NO: 12 - DNA, 21 bp, Artificial sequence
  514.7f primer
gacatgattctacataggagg

SEQ ID NO: 13 - DNA, 21 bp, Artificial sequence
  514.7r primer
gaccacaaaaaacaagactgc

SEQ ID NO: 14 - DNA, 26 bp, Artificial sequence
  63.1 f primer
aaataaatacagaggaaaatttttgc

SEQ ID NO: 15 - DNA, 20 bp, Artificial sequence
  63.1 r primer
tctgcatatgcgtttactgc

SEQ ID NO: 16 - DNA, 24 bp, Artificial sequence
  zf anchor f primer
tgtaccactttaaatttgtgatgc

SEQ ID NO: 17 - DNA, 20 bp, Artificial sequence
  zf anchor r primer
tcaaacaaacaaaatgacgc

SEQ ID NO: 18 - DNA, 20 bp, Artificial sequence
  a fragment f primer
acttgttcaccgttcaaacg

SEQ ID NO: 19 - DNA, 24 bp, Artificial sequence
  a fragment r primer
atttatgattatgtggatcaaacc

SEQ ID NO: 20 - DNA, 20 bp, Artificial sequence
  ATG+ 1600 primer
gaacaccctgaaacatttcc

SEQ ID NO: 21 - DNA, 21 bp, Artificial sequence
  ATG+ 2100 primer
ggcaagccaattatgtatacc

SEQ ID NO: 22 - DNA, 20 bp, Artificial sequence
  a-b for primer
acaaccaaacaaccctttgc

SEQ ID NO: 23 - DNA, 20 bp, Artificial sequence
  a-b rev primer
taccgttacttggtcactcc

SEQ ID NO: 24 - DNA, 20 bp, Artificial sequence
  c fragment f primer
attcttggactagactctgc

SEQ ID NO: 25 - DNA, 20 bp, Artificial sequence
  c fragment r primer
ccttttcacactacccttcg

SEQ ID NO: 26 - DNA, 20 bp, Artificial sequence
  d fragment f primer
aaaggtcatggagacatacc

SEQ ID NO: 27 - DNA, 20 bp, Artificial sequence
  d fragment r primer
cagacgtaacgttaacatcg

SEQ ID NO: 28 - DNA, 27 bp, Artificial sequence
  TMF seqf primer
aaatagtaataatagggaaaaataggg

SEQ ID NO: 29 - DNA, 20 bp, Artificial sequence
  TMF seqr primer
acctctcttctctctctccc

SEQ ID NO: 30 - DNA, 26 bp, Artificial sequence
  Control for gDNA f primer
gaaatattccaataataatttggacc

SEQ ID NO: 31 - DNA, 23 bp, Artificial sequence
  Control for gDNA r primer
ttcttctcaaaccatttaattcc

SEQ ID NO: 32 - DNA, 37 bp, Artificial sequence
  hi-TAIL 0 primer
ggttcttataacctactccctagctcctctattaccc

SEQ ID NO: 33 - DNA, 50 bp, Artificial sequence
  hi-TAIL 1 primer
acgatggactccagtccggcctctctcccaaataaaagatcatcaaatcg

SEQ ID NO: 34 - DNA, 26 bp, Artificial sequence
  hi-TAIL 2 primer
taacaattcgatgacgatgttagcgg

SEQ ID NO: 35 - DNA, 22 bp, Artificial sequence
  Solyc09g090190 rtf primer
ttttgtacctggtcaactaagc

SEQ ID NO: 36 - DNA, 25 bp, Artificial sequence
  Solyc09g090190 rtr primer
gagaataaggttatacgttttgagg

SEQ ID NO: 37 - DNA, 20 bp, Artificial sequence
  Ubiquitin f primer
cgtggtggtgctaagaagag

SEQ ID NO: 38 - DNA, 20 bp, Artificial sequence
  Ubiquitin r primer
acgaagcctctgaacctttc

SEQ ID NO: 39 - DNA, 26 bp, Artificial sequence
  TMFgs primer
caccatggaacacaaccaagaagtgg

SEQ ID NO: 40 - DNA, 21 bp, Artificial sequence
  TMFr primer
ttagcttgaatttccatttgg

SEQ ID NO: 41 - DNA, 27 bp, Artificial sequence
  pS PstI-F primer
aaactgcagctatcaaggatttttcaa

SEQ ID NO: 42 - DNA, 29 bp, Artificial sequence
  pS BamHI-R primer
aaaggatccatttgatgaggatgaagaag

SEQ ID NO: 43 - DNA, 29 bp, Artificial sequence
  ANCDS XhoI-F primer
aaactcgagatggaagcttttcatcatcc

SEQ ID NO: 44 - DNA, 29 bp, Artificial sequence
  ANCDS KpnI-R primer
aaaggtacctcagttgaatgactgaaagg

SEQ ID NO: 45 - DNA, 627 bp, Arabidopsis thaliana
atggaacacaaccaagaagtggattcaccaaactcagtcatcatcaaccaccatcatcatcacaatcacaaccttgataacaattcgatgacgatgttagcgggtaacaataataataataataataatagttatcttgcttcatcgtcatcgaattcgccaacaaccctaagccgatacgagaaccaaaaacgtcgtgattggaacacgtttggtcaatacctaagaaaccatagaccaccactctctctcactcgttgtagtggggcacacgtgctagaatttcttcgataccttgaccaatttggaaagactaaggttcacactcaactatgtccatttttcgggcacccaaacccacccgcaccttgtccgtgcccgttaagacaagcctgggggagccttgatgcactcataggacggcttcgagccgcttatgaggaaaacggtggaaaacccgaaactaacccttttggtgcaagggcagttaggctttaccttcgcgaagttcgcgattctcaagctaaagctagaggaataagttatgaaaaaaagaaacgcaaaaaaccaaatcctcaacattcatcatcgtcatcattgccaccaccaaatggaaattcaagctaa

SEQ ID NO: 46 - protein, 208 aa, Solanum lycopersicum
MEHNQEVDSPNSVIINHHHHHNHNLDNNSMTMLAGNNNNNNNNSYLASSSSNSPTTLSRYENQKRRDWNTFGQYLRNHRPPLSLTRCSGAHVLEFLRYLDQFGKTKVHTQLCPFFGHPNPPAPCPCPLRQAWGSLDALIGRLRAAYEENGGKPETNPFGARAVRLYLREVRDSQAKARGISYEKKKRKKPNPQHSSSSSLPPPNGNSS

SEQ ID NO: 47 - protein, 171 aa, Solanum lycopersicum
MDSFVEVEPSNTTTNNNNITSSSSTSSSRYENQKRRDWNTFGQYLKNHRPPLSLSRCSGAHVLEFLRYLDQFGKTKIHTLICPFYGLPNPPAPCPCPLRQAWGSLDALIGRLRAAYEENGGNPEMNPFGTRAVRLYLREVRDLQSKARGVSYEKKKRKRPSQPSPPPLQSG

SEQ ID NO: 48 - protein, 218 aa, Solanum lycopersicum
MASFTELVESSNHHHHEKINIETVNNIEIISVSASSSSSAATPAPASSSSRYENQKRRDWNTFGQYLRNHRPPLTLSRCSGAHVLEFLRYLDQFGKTKVHTPMCPFYGHPNPPAPCPCPLKQAWGSLDALVGRLRAAYEENGGKPETNPFGARAVRLYLREVRDLQSKARGVSYEKKKKRKRPTPPPPIPPPQLLSTSVQLPLSSPPPSGKGALPFEL

SEQ ID NO: 49 - protein, 222 aa, Solanum lycopersicum
MDSTSRVEQPDPNIVGSSEGGTGTSSASAVTEGGQSTTVSAAPPSRYESQKRRDWNTFLQYLRNHKPPLTLARCSGAHVIEFLKYLDQFGKTKVHVTGCPYFGHPNPPAPCACPLKQAWGSLDALIGRLRAAYEENGGKPESNPFGAKAVRIYLREVRESQAKARGIPYEKKKRKRPSTSSSVATAGSAVAAEGGSSSGGGDGSGGDGVIGQQPPTDPNTTV

SEQ ID NO: 50 - protein, 181 aa, Solanum lycopersicum
MLDVYSTINSVSQNFSLSSAPAPTLPLPPPSSPPTVSRYELQKRRDWNTFGQYLRNHKPPLILARCSGANILEFLKYLDQFGKTKVHSCNCPFFGDPHPPAPCNCPLKQAWGSLDALIGRLRAAFEENGGRTETNPFGARAVRLYLKEVRDTQAKARGIAYEKKKRRNIKQRISSTINNCD

SEQ ID NO: 51 - protein, 211 aa, Solanum lycopersicum
MDFVTAQGNNFTTSGNNMVQGTNFIANSTTMIESSVPPLSRYENQKRRDWNTFCQYVRNHQPPLSLPQCTSAHILEFLRYLDQFGKTKVHNQNCPFFGLLNPPAPCPCPLRQAWGSLDALIGRLRAAYEENGGKPEMNPFGSRNVRLFLREVRDFQSKSRGVSYEKKRKRTTSSTNNNKSKIITVIDGGGDGCGTGTCATFCGYGNIGNGN

SEQ ID NO: 52 - protein, 192 aa, Solanum lycopersicum
MNPSTIVMTKELSAGSSRSGGEQLQNNNPAPLSRYESQKRRDWNTFGQYLKNQRPPVPLSQCNCNHVLEFLRYLDQFGKTKVHLHGCVFFGQPDPPAPCTCPLRQAWGSLDALIGRLRAAYEENGGSPENNPFGNGAIRLYLREVKECQAKARGIPYKKKKKRKLNNNSIKPIGAGVGASADQHKNLMQANI

SEQ ID NO: 53 - protein, 205 aa, Solanum lycopersicum
MSNDQIIIEGEGGGGGGEGSSSRSKTTILIAPSDDHHHHHQLPPVPPQLSRYESQKRRDWNTFGQYLKNQRPPVPLSQCNYNHVLEFLRYLDQFGKTKVHLHGCPFFGQPEPPGPCTCPLRQAWGSLDALIGRLRAAYEENGGLPENNPFASGAIRVYLREVRDFQAKARGICYKKKKKKRKMQNKPTSSNAHEPTTTTFQFQSS

SEQ ID NO: 54 - protein, 180 aa, Solanum lycopersicum
MDVANDNPSNDVVDILSTPRLSRYESQKRRDWNTFCQYIRNHHPLMSLLQCSSIHVLEFLRYLDQFGKTKVHNSNCPFFGMINPPAPCACPLRQAWGSLDALIGRLRAAYEEHGGNSEMNPFGARSIKLFLRDVRNFQSKSRGISYDKKRKRSKRNHKNIMEMKEVDHQIHDDKNVGANL

SEQ ID NO: 55 - protein, 213 aa, Solanum lycopersicum
MMSSEKIREVGEGSSSSGGAISIIATPLNNHHRQSSSSSLSTLAPTPASSSAPQLSRYESQKRRDWNTFGQYLKNQRPPISLPQCNYNHVLDFLRYLDQFGKTKVHLHGCLFFGQPEPPGPCTCPLRQAWGSLDALIGRLRAAYEENGGLQETNPFASGAIRVYLREVRDSQAKARGIPYKKKKKKKRPNLQIKASNNNDGATSANFQLQSTT

SEQ ID NO: 56 - protein, 178 aa, Solanum lycopersicum
MSSSDIRGKDLAEGSSRSPGRDQPPSRYESQKRRDWNTFNHYLKNQRPPILLPHCHSNHVLEFLRYLDQFGKTKVHLLGCMFYGQPDPPAPCTCPLRQAWGSLDALIGRLRAAYEENGGSSETNPFASVGIRVYLREVKECQAKARGIAYKKKQKKLANSPSKGDHDDASCPGFLTFS

SEQ ID NO: 57 - protein, 147 aa, Solanum lycopersicum
MTLTQSDHHHQLSPPPQLSRYESQKRRDWNTFGQYLKNHKPPVPLPQCNYNHVLDFLRYLDQFGKTKVHLNGCVFFGQVEQVGPCTCPLRQAWGSLDALIGRLRAAYEENGGLQETNPFANSAIRIYLREARADEDLKLRINSIAFA

SEQ ID NO: 58 - protein, 153 aa, Selaginella moellendorffii
GGASTSRADANASSSQQAPPSRYEAQKRRDWNTFGQYLKNHRPPLALSRCSGAHVLEFLRYLDQFGKTKIHAPACPFFGLAHPPAPCACPLRQAWGSLDALIGRLRAAFEEHGGKPESNPFGARAVRLYLREVREMQAKARGIAYEKKKRKRP

SEQ ID NO: 59 - protein, 173 aa, Selaginella moellendorffii
SSGDGGAGGGAAAAAGAGEGSATAPPSRYEAQKRRDWNTFGQYLRNHRPPLTLPRCSGANVLEFLRYLDQFGKTKIHAPACPFFGIAHPPAPCACPLRQAWGSLDALIGRLRAAFEEHGGKPESNPFGARAVRLYLREVREMQAKARGIAYEKKKRKRPSSNNTGAANAGTSS

SEQ ID NO: 60 - protein, 261 aa, Oryza sativa
MAKHTRKSFISFEPDYARFMHHHMKNASCTSFHSLTYTTRMGDTPGYEQKVYVVCFYHSVNYRVFQGNTLQQLLLRSVHLEHWGTPGYWSITLANMARTAAGRVERGGGRGGRACGRRSHPSSPAPWPCPLRQAWGSLDVLVGRLRTAFDEHGGHPEANPFGARVVRLYLREVCDSQAKVRGIAYEKKRRKRPPTSSSHSQDGTAATCPASPAASPTPLPPPPERSADMGACVAIVVAVGCTPLSLAARRGCSYCALARRR

SEQ ID NO: 61 - protein, 212 aa, Oryza sativa
MDPSGPGPSSAAAGGAPAVAAAPQPPAQLSRYESQKRRDWNTFLQYLRNHRPPLTLARCSGAHVIEFLRYLDQFGKTKVHASGCAFYGQPSPPGPCPCPLRQAWGSLDALIGRLRAAYEESGGTPESNPFAARAVRIYLREVRDSQAKARGIPYEKKKRKRSQAAQPAGVEPSGSSSAAAAAAGGGDAGSGGGAAATTTAQPGGSGTAPSAS

SEQ ID NO: 62 - protein, 209 aa, Oryza sativa
MELSPPNHESSPPTAGGGGGGGGDGAGGSSSAGASSSAGGGAATPQTPSRYEAQKRRDWNTFGQYLRNHRPPLGLAQCSGAHVLEFLRYLDQFGKTKVHTAACPFFGHPNPPAPCPCPLRQAWGSLDALVGRLRAAFEENGGRPESNPFAVRAVRLYLREVREHQARARGVSYEKKKRKKPQPADTSGGGGHPHPPPPPPPPPSAGAAC

SEQ ID NO: 63 - protein, 248 aa, Oryza sativa
MDRHHHHHHHHHHHMMSGGGQDPAAGDGGAGGATQDSFFLGPAAAAMFSGAGSSSSGAGTSAGGGGGGPSPSSSSPSLSRYESQKRRDWNTFGQYLRNHRPPLSLSRCSGAHVLEFLKYMDQFGKTKVHTPVCPFYGHPNPPAPCPCPLRQAWGSLDALIGRLRAAYEENGGTPEMNPFGARAVRLYLREVRETQARARGISYEKKKRKKPSSAGAGAGPSSEGSPPPPGGSASGGGDTSASPQFIIP

SEQ ID NO: 64 - protein, 270 aa, Oryza sativa
MDMIGMASPAESPGGGGTARPSRYESQKRRDWQTFGQYLRNHRPPLELSRCSGAHVLEFLRYLDQFGKTKVHAHGCPFFGHPSPPAPCPCPLRQAWGSLDALVGRLRAAFEEHGGRPESNPFGARAVRLYLRDIRDTQSKARGIAYEKKRRKRAAASHTKQKQQQQQLVEQAVAPPAAAAAAAALPDMETTTTTTTVPHFLFPAHFLHGHYFLAPAGEQPGGGDVAASTGGAAGAPSGGGGEDLVLAMAAAAAAAEAHAAGCMMPLSVFN

SEQ ID NO: 65 - protein, 202 aa, Oryza sativa
MDLSPNPDSPPSGGGNGGGGGSSSSNSSPSMGAGAPQSPSRYEAQKRRDWNTFGQYLRNHRPPLSLAQCSGAHVLEFLRYLDQFGKTKVHTAACPFFGHPSPPAPCPCPLRQAWGSLDALVGRLRAAFEENGGRPESNPFAARAVRLYLREVREHQARARGVSYEKKKRKKPQQQQLQGGDSSGLHGHQHHPPPPPPAGAAC

SEQ ID NO: 66 - protein, 284 aa, Oryza sativa
MEPSPDAPRAGAAEEQPGPSSSASAPAPAASSNEEEGRHQSQAQQQVQEAQPQPLAQQAPAAAGLSRYESQKRRDWNTFLQYLRNHKPPLTLPRCSGAHVIEFLKYLDQFGKTKVHADGCAYFGEPNPPAPCACPLRQAWGSLDALIGRLRAAYEESGGRPESNPFAARAVRIYLREVREAQAKARGIPYEKKRKRGAAAAAAAPPVVVAPPPVVTAPDDATGTSGGAGEDDDDDEATHSGEQQDTTPAASPTTPPATSVGTTTAAATAAAAKGSAAKGSATSS

SEQ ID NO: 67 - protein, 238 aa, Oryza sativa
MEGGGGGADGQAQPVAQAPPAMQPMQQLSRYESQKRRDWNTFLQYLKNHRPPLTLARCSGAHVIEFLKYLDQFGKTKVHASGCAYYGQPSPPAPCPCPLRQAWGSLDALIGRLRAAYEESGHAPESNPFAARAVRIYLREVRDAQAKARGIPYEKKKRKRTQQQQPPPPPPPPPQHQPGAAAGEASSSSSAAAAAVAAEGSGSSAAAAAATSQTGGGGGGSTTTTTASAAAPTTATRV

SEQ ID NO: 68 - protein, 277 aa, Oryza sativa
MQGGGGGDSSGGGGGEAPRPSRYESQKRRDWHTFGQYLRNHRPPLELSRCSGAHVLEFLRYLDQFGKTKVHAAGCPFFGHPSPPAPCPCPLRQAWGSLDALVGRLRAAFEEHGGRPEANPFGARAVRLYLREVRDSQAKARGIAYEKKRRKRPPTSSSSSQAAAAAAAATSPASPAASPTPPPPPPTERSADVRPMPPEGHFFIPHPHFMHGHFLVPGGDADHHHQVSNAGNGGNTNTNTNTNTGGGGGNGDEMAVAMAAVAEAHAAGCMLPLSVFN

SEQ ID NO: 69 - protein, 276 aa, Oryza sativa
MSSSSAAALGSDDGCSPAELRPSRYESQKRRDWQTFTQYLAAHRPPLELRRCSGAHVLEFLRYLDRFGKTRVHEPPCPSYGGRSPSAAGPVAAAAAACQCPLRQAWGSLDALVGRLRAAYDERHGRAGEPDAVAGAGAVATDSTSSSSAAAANPFAARAVRLYLRDVRDAQAMARGISYHKKKKRRGGNMNGARGGGGGGARAGVNDGDATAPPVAVTPGLPLPPLPPCLNGVPFEYCDFGSVLGGAHGAHGGHGGGGGGFYGAGVYLPFLYNTFS

SEQ ID NO: 70 - protein, 204 aa, Oryza sativa
MEFVAHAAAPDSPHSDSGGGGGGMATGATSASAAGASPSRYESQKRRDWNTFGQYLRNHRPPLSLARCSGAHVLEFLRYLDQFGKTKVHAPACPFFGHPAPPAPCPCPLRQAWGSLDALVGRLRAAYEENGGRPENNPFGARAVRLYLREVREHQARARGVSYEKKKRKKPPHPSSAAAAHDDAANGALHHHHHMPPPPPGAAA

SEQ ID NO: 71 - protein, 289 aa, Physcomitrella patens
MTSDFRNSEFRSILNPTVATLHPVICSRPGEPSFIRDEHFIRSLDRDFKPATIRENYDFASRIQSLMSGEVALGNLQDAHGGAGPSSLMESSGLVPNGLIPLGMQVDPQHDQNSRAPSRYEAQKRRDWNTFGQYLRNHRPPLALARCTGVHVLEFVRYLDQFGKTKVHVQSCPFFGLPHPPHPCPCPLRQAWGSLDALIGRLRAAFEENGGKPESNPFGARQVRLYLREVREMQAKARGIAYEKKKRKRMPNAASDSSQNGVNGVNGINLSGVVGGMNNGNVHGMPTQQ

SEQ ID NO: 72 - protein, 289 aa, Physcomitrella patens
MMSEFHSSEFRNLLNIPPASSIHPGDSSRFNDTTAFQFLPGEQYINALHRDSKPFMTRDNDFASRIQSLMNGEVALDNNLEHNSAGPSSGFMRLPGQAPNGTVSSREQVDHQHEQNTGVPSRYEAQKRRDWNTFGQYLRNHRPPLALARCTGLHVLEFVRYLDQFGKTKVHIASCSFFGLPHPPHPCPCPLRQAWGSLDALIGRLRAAFEENGGKPESNPFGARQVRLYLREVREMQAKARGIAYEKKKRKRIPPPSELGGNGVNVGMIVNIGAGGMSNGNVHPLPAQQ

SEQ ID NO: 73 - protein, 292 aa, Physcomitrella patens
MTSNFRNAEFRNILNPTASTIHPGNYSRPGEPQFVREDHFISPLHRDSKPVTIRENHDFSSRIQSLMSGEVALGNLQDAHGGGVPSTLMGSPRLVPNGVIPQGMQVDAQHDQNSRAPSRYEAQKRRDWNTFGQYLRNHRPPLALARCTGVHVLEFVHYLDQFGKTKVHVPSCPFFGLPHPPHPCPCPLRQAWGSLDALIGRLRAAFEENGGKPESNPFGARQVRLYLREVREMQAKARGIAYEKKKRKRMPNAGSGGSQGGVNGMNGVNGMNLSNGVGGMTNGNVHAMPAQQ

SEQ ID NO: 74 - protein, 289 aa, Physcomitrella patens
MTSDFRSSEFINLLNVPTAPGIHPSDHNRFSETMPFQFRPEEQYVYSMHRDTKPATSTRENDFANRIQSFMNGGVALSNNVNRGDAGPSSGFMRSVGPVLDGMASPPTQVDNQHEQNTGAPSRYEAQKRRDWNTFGQYLRNHRPPLPLARCTGAHVLEFMRYLDQFGKTKVHIASCSFFGLPHPPHPCPCPLRQAWGSLDALIGRLRAAFEENGGMPESNPFGARQVRLYLREVREMQAKARGIAYEKKKRKRMPPPSTLGVNGVIANGTSNGVGGVNNGDVHPLRSLQ

SEQ ID NO: 75 - protein, 219 aa, Arabidopsis thaliana
MDMIPQLMEGSSAYGGVTNLNIISNNSSSVTGATGGEATQPLSSSSSPSANSSRYENQKRRDWNTFGQYLRNHRPPLSLSRCSGAHVLEFLRYLDQFGKTKVHTNICHFYGHPNPPAPCPCPLRQAWGSLDALIGRLRAAFEENGGKPETNPFGARAVRLYLREVRDMQSKARGVSYEKKKRKRPLPSSSTSSSSAVASHQQFQMLPGTSSTTQLKFEK

SEQ ID NO: 76 - protein, 177 aa, Arabidopsis thaliana
MSSPRERGKSLMESSGSEPPVTPSRYESQKRRDWNTFGQYLKNQRPPVPMSHCSCNHVLDFLRYLDQFGKTKVHVPGCMFYGQPEPPAPCTCPLRQAWGSLDALIGRLRAAYEENGGPPETNPFASGAIRVYLREVRECQAKARGIPYKKKKKKKPTPEMGGGREDSSSSSSSFSFS

SEQ ID NO: 77 - protein, 191 aa, Arabidopsis thaliana
MSSDRHTPTKDPPDHPSSSSNHHKQPLPPQPQQPLSRYESQKRRDWNTFVQYLKSQNPPLMMSQFDYTHVLSFLRYLDQFGKTKVHHQACVFFGQPDPPGPCTCPLKQAWGSLDALIGRLRAAYEEHGGGSPDTNPFANGSIRVHLREVRESQAKARGIPYRKKKRRKTKNEVVVVKKDVANSSTPNQSFT

SEQ ID NO: 78 - protein, 195 aa, Arabidopsis thaliana
MASHSNKGKGIAEGSSQPQSQPQPQPHQPQSPPNPPALSRYESQKRRDWNTFCQYLRNQQPPVHISQCGSNHILDFLQYLDQFGKTKVHIHGCVFFGQVEPAGQCNCPLKQAWGSLDALIGRLRAAFEENGGLPERNPFAGGGIRVFLREVRDSQAKARGVPYKKRKKRKKRNPMKSHDGEDGTTGTSSSSNLAS

SEQ ID NO: 79 - protein, 196 aa, Arabidopsis thaliana
MESADSGRSDPVKGDDPGPSFVSSPPATPSRYESQKRRDWNTFLQYLKNHKPPLALSRCSGAHVIEFLKYLDQFGKTKVHVAACPYFGHQQPPSPCSCPLKQAWGSLDALIGRLRAAYEENGGRPDSNPFAARAVRIYLREVRESQAKARGIPYEKKKRKRPPTVTTVRVDVASSRQSDGDPCNVGAPSVAEAVPP

SEQ ID NO: 80 - protein, 164 aa, Arabidopsis thaliana
MTSTNTRNKGKCIVEGPPPTLSRYESQKSRDWNTFCQYLMTKMPPVHVWECESNHILDFLQSRDQFGKTKVHIQGCVFFGQKEPPGECNCPLKQAWGSLDALIGRLRAAYEENGGLTEKNPFARGGIRIFLREVRGSQAKARGVLYKKKKRLVVVGTGTSTTWT

SEQ ID NO: 81 - protein, 195 aa, Arabidopsis thaliana
MDHIIGFMGTTNMSHNTNLMIAAAATTTTTSSSSSSSSGGSGTNQLSRYENQKRRDWNTFGQYLRNHRPPLSLSRCSGAHVLEFLRYLDQFGKTKVHTHLCPFFGHPNPPAPCACPLRQAWGSLDALIGRLRAAFEENGGSPETNPFGARAVRLYLREVRDSQAKARGISYEKKKRKRPPPPLPPAQPAISSSPN

SEQ ID NO: 82 - protein, 201 aa, Arabidopsis thaliana
MDLISQNHNNRNPNTSLSTQTPSSFSSPPSSSRYENQKRRDWNTFCQYLRNHHPPLSLASCSGAHVLDFLRYLDQFGKTKVHHQNCAFFGLPNPPAPCPCPLRQAWGSLDALIGRLRAAYEENGGAPETSPFGSRSVRIFLREVRDFQAKSRGVSYEKKRKRVNNKQITQSQPQSQPPLPQQPQQEQGQSMMANYHHGATQ

SEQ ID NO: 83 - protein, 190 aa, Arabidopsis thaliana
MDLISHQPNKNPNSSTQLTPPSSSRYENQKRRDWNTFCQYLRNHRPPLSLPSCSGAHVLEFLRYLDQFGKTKVHHQNCAFFGLPNPPAPCPCPLRQAWGSLDALIGRLRAAYEENGGPPEANPFGSRAVRLFLREVRDFQAKARGVSYEKKRKRVNRQKPQTQPPLQLQQQQQQPQQGQSMMANYSGATV

SEQ ID NO: 84 - protein, 182 aa, Arabidopsis thaliana
MEGETAAKAAASSSSSPSRYESQKRRDWNTFLQYLRNHKPPLNLSRCSGAHVLEFLKYLDQFGKTKVHATACPFFGQPNPPSQCTCPLKQAWGSLDALIGRLRAAFEEIGGGLPESNPFAAKAVRIYLKEVRQTQAKARGIPYDKKKRKRPHTDTATPIAGDGDDAEGSGGAALVVTAATTV

SEQ ID NO: 85 - protein, 1309 aa, Arabidopsis thaliana
MAASFCGSRRYDVFPSFSKVDVRRSFLAHLLKELDRRLINTFTDHGMERNLPIDAELLSAIAESRISIVIFSKNYASSTWCLDELVEIHTCYKELAQIVVPVFFNVHPSQVKKQTGEFGKVFGKTCKGKPENRKLRWMQALAAVANIAGYDLQNWPDEAVMIEMVADDVSKKLFKSSNDFSDIVGIEAHLEAMSSILRLKSEKARMVGISGPSGIGKTTIAKALFSKLSPQFHLRAFVTYKRTNQDDYDMKLCWIEKFLSEILGQKDLKVLDLGAVEQSLMHKKVLIILDDVDDLELLKTLVGQTGWFGFGSRIVVITQDRQLLKAHDINLIYEVAFPSAHLALEIFCQSAFGKIYPPSDFRELSVEFAYLAGNLPLDLRVLGLAMKGKHREEWIEMLPRLRNDLDGKFKKTLRNYLPVIRKRVSNEEGGREKLKKGNKKLDLDEEFPGGEIYSDEIPSPTSNWKDTDDFDSGDIIPIIADKSTTIIPNRRHSNDDWCSFCEFLRNRIPPLNPFKCSANDVIDFLRTRQVLGSTEALVDRLIFSSEAFGIKPEENPFRSQAVTSYLKAARDMTREKECILVFSCHDNLDVDETSFIEAISKELHKQGFIPLTYNLLGRENLDEEMLYGSRVGIMILSSSYVSSRQSLDHLVAVMEHWKTTDLVIIPIYFKVRLSDICGLKGRFEAAFLQLHMSLQEDRVQKWKAAMSEIVSIGGHEWTKGSQFILAEEVVRNASLRLYLKSSKNLLGILALLNHSQSTDVEIMGIWGIAGIGKTSIAREIFELHAPHYDFCYFLQDFHLMCQMKRPRQLREDFISKLFGEEKGLGASDVKPSFMRDWFHKKTILLVLDDVSNARDAEAVIGGFGWFSHGHRIILTSRSKQVLVQCKVKKPYEIQKLSDFESFRLCKQYLDGENPVISELISCSSGIPLALKLLVSSVSKQYITNMKDHLQSLRKDPPTQIQEAFRRSFDGLDENEKNIFLDLACFFRGQSKDYAVLLLDACGFFTYMGICELIDESLISLVDNKIEMPIPFQDMGRIIVHEEDEDPCERSRLWDSKDIVDVLTNNSGTEAIEGIFLDASDLTCELSPTVFGKMYNLRLLKFYCSTSGNQCKLTLPHGLDTLPDELSLLHWENYPLVYLPQKFNPVNLVELNMPYSNMEKLWEGKKNLEKLKNIKLSHSRELTDILMLSEALNLEHIDLEGCTSLIDVSMSIPCCGKLVSLNMKDCSRLRSLPSMVDLTTLKLLNLSGCSEFEDIQDFAPNLEEIYLAGTSIRELPLSIRNLTELVTLDLENCERLQEMPSLPVEIIRRT

SEQ ID NO: 86 - protein, 225 aa, Zea mays
MDPSGPAGPSSAAGSGDDALAPPQHQVQPLPQAQPQPQQAAPPPQLSRYESQKRRDWNTFLQYLRNHRPPLTLARCSGAHVIEFLKYLDQFGKTKVHAAGCAYYGQPNPPAPCPCPLRQAWGSLDALIGRLRAAYEESGGTPESNPFAARAVRIYLREVRDSQAKARGIPYEKKKRKRTQQAAAEASTSSSAAAAAGGSGSGSGRAAAASSAAQAGGSSAAPSTT

SEQ ID NO: 87 - protein, 279 aa, Zea mays
MQMQVGGGAADSPGAAAAAGAEAPRPSRYESQKRRDWHTFGQYLRNHRPPLELARCSGAHVLEFLRYLDQFGKTKVHAPGCPFFGHPSPPAPCPCPLRQAWGSLDALIGRLRAAFEEHGGRPEANPFGARAVRLYLREVRDSQAKARGIAYEKKRRKRPSASSQSSPQAATTPSLQAPPVSTPALSDAAAERADVRAHVPDAGHGHQHQHHFFMPHPQFLHGFRLLPGNPEAVAGNGNGDGSSSSASVAAGSGDEIALAMAAAAEAHAAGCMLPLSVFN

SEQ ID NO: 88 - protein, 213 aa, Zea mays
MDLVPQLDSPHSDNGGGGGSGSLASGALSPGASSAGTVSALASPSRYESQKRRDWNTFGQYLRNHRPPLSLARCSGAHVLEFLRYLDQFGKTKVHTAACPFFGHPAPPAPCPCPLRQAWGSLDALVGRLRAAYEENGGRPENNPFGARAVRLYLREVRDHQSRARGVSYEKKKKRKKAPPHPVPAAVISSSHDGNGHHYEHQMPPPPPPGAAA

SEQ ID NO: 89 - protein, 209 aa, Zea mays
MDPSGPGPSSVVGAGGGEAPAVAPQRPAQLSRYESQKRRDWNTFLQYLRNHRPPLTLARCSGAHVIEFLRYLDQFGKTKVHAAGCAYYGQPAPPGPCPCPLRQAWGSLDALIGRLRAAYEESGGTPESNPFAARAVRIYLREVRDSQAKARGIPYEKKKRKRTQQAAAEASTSSSAAAAAGGSGSGSGRAAAASSAAQAGGSSAAPSTT

SEQ ID NO: 90 - protein, 246 aa, Zea mays
MDHHHHHHHHMIPGQEPPAADSSAPDSFFLGPAGAIIFSGGAGVSGAGSSSGAAALGSSAGAGGGPSPSSSSPSLSRYESQKRRDWNTFGQYLRNHRPPLSLSRCSGAHVLEFLKYMDQFGKTKVHTPVCPFYGHPNPPAPCPCPLRQAWGSLDALIGRLRAAYEENGGTPEMNPFGARAVRLYLREVRESQARARGISYEKKKRKKPSAASAAAPGPSSEGSPPPGPSGGGGGFGTSASPRFIMP

SEQ ID NO: 91 - protein, 210 aa, Zea mays
MDLVPHPDSPHSDNSGGGGGSASGALSPGASSAGAASALASPSRYESQKRRDWNTFGQYLRNHRPPLSLARCSGAHVLEFLRYLDQFGKTKVHTPACPFFGHPAPPAPCPCPLRQAWGSLDALVGRLRAAYEENGGRPENNPFGARAVRLYLREVRDHQSRARGVSYEKKKRKKAPAHPVPAAVISSSHDGNGHHYEHQMPPPPPPGAAA

SEQ ID NO: 92 - protein, 270 aa, Zea mays
MRRADLVELVFALRGDRQVTERAMEVAGVVASAADSPGAAAARPSRYESQKRRDWHTFGQYLRNHRPPLELARCSGAHVLEFLRYLDQFGKTKVHAARCPFFGHPSPPAPCPCPLKQAWGSLDALVGRLRAAFEEHGGRPEANPFGARAVRLYLREVRDSQAKARGIAYEKKRRKRHPPAHRQPKQQQQQDGQHQHPSHAAPGTVAEPPAPHFLIPHAHFLHGHFLAPATEPIDPAAGGGGGTGDDIALAMAAAAEAHAAGFLMPLSVFN

SEQ ID NO: 93 - protein, 247 aa, Zea mays
MDHHHHHHHHHHHHMIPGQEPSATDGAAPDSFFLGPAAAVIFSGGAGAGSSSSGAAALGSSAGGGGGPSPSSSSPSLSRYESQKRRDWNTFGQYLRNHRPPLSLSRCSGAHVLEFLKYMDQFGKTKVHTPVCPFYGHPNPPAPCPCPLRQAWGSLDALIGRLRAAYEENGGTPEMNPFGARAVRLYLREVRETQARARGISYEKKKRKKPSAASAAAAGPSSEGSPPPGPSSGGGGPDTSSPQFIMP

SEQ ID NO: 94 - protein, 278 aa, Zea mays
MQMHVGGGAADSPEATRPSRYESQKRRDWHTFGQYLRNHRPPLELARCSGAHVLEFLRYLDQFGKTKVHAAGCPFFGHPSPPAPCPCPLRQAWGSLDALVGRLRAAFEEHGGRPEANPFGARAVRLYLREVRDSQAKARGIAYEKKRRKRPSSFASASSQSSPQAATSPPQQAAPASSPILSDAAAERADVRAFVSDAGARGTGTGTSNSTTSSCRTRSSCTGSACCRETTEAVAGNGNGSSSSASVAASSGDEIALALAAAEEAHAAGCMLPLSVFN

SEQ ID NO: 95 - protein, 301 aa, Zea mays
MEPGPDAPAGGGGGGGTSEPAEAGPSPSSSSAAAAASSSSRQQAEQEAQQQQGAQQREQPAVRAQAQPQPQPLTQQPPAGLSRYESQKRRDWNTFLQYLRNHKPPLTLARCSGAHVIEFLRYLDQFGKTKVHAEGCAYFGQPNPPAPCTCPLRQAWGSLDALIGRLRAAYEESGGRPESNPFAAKAVRIYLRDVREAQAKARGIPYEKKKRKRGSAAAPPVAPPPVVTAEAAGTSSGAGGGDDEDDDEPSPSADEPQRQQTATPAPPASIISSASASSSSVAPATTTTTSKKEKEGSAPSS

SEQ ID NO: 96 - protein, 256 aa, Zea mays
MSTSGARSWSPPPRPSRYESQKRRDWQTFTRYLGAHRPPLELCRCSGAHVLEFLRYLDRFGKTRVHAPSCAAYGGGGGGRPVEAAVACQCPLRQAWGSLDALVGRLRAAFEERHGARGIWTSSQQPDGGVGVGVGGGDGANPFAARAVRLYLRDVRDAQSRARGISYSRKKKKRSKQQDGAAAAGCARPPVTSVTLMPAAALPHAPPPPCPPPPLPPPYCLTGVPFEFCDYGSVLGGVTANGAPGFYLPSLFNTFG

SEQ ID NO: 97 - protein, 316 aa, Zea mays
MEPGPDGPAGGQGTSAPAEAGPSPSSSSAAAAASSSSRKQAEQQAPPQQQAGAQQRQQQAARAQHQEAPAPQAAQAQPQPLAQQPPPPPPPAGLSRYESQKRRDWNTFLQYLRNHKPPLTLARCSGAHVIEFLRYLDQFGKTKVHTEGCAHFGQPNPPAPCACPLRQAWGSLDALIGRLRAAYEESGGRPESNPFAAKAVRIYLRDVREAQAKARGIPYEKRKRKRGSAAAAPPVAPPPVVTAETAGTTSGTVCDEEEEPSPSAGDEPQKQTTTPTSASAPPTSTSSASASSSSAAAATTSTTTTRKEEEGSAPSS

SEQ ID NO: 98 - protein, 247 aa, Zea mays
MDPSGPAGAGAGPSSAAGAGGDDAHAHAPPQQHQIQPLAQAQAQPQPHQLSRYESQKRRDWNTFLQYLQNHRPPLTLARCSGAHVIEFLKYLDQFGKTKVHAAGCAHFGQPSPPAPCPCPLHQAWGSLDALIGRLRAAYEESGHAPESNPFAARAVRIYLRDVRDAQAKARGIPYEKKSRKRKQPPPAAAAGEAAAASSSSAAAARVAAGSAGDGSSASGSAAAKAAPTTAQGSAAADAATSTSRVQ

SEQ ID NO: 99 - protein, 208 aa, Zea mays
MDPSGPGPSSVIGAAGGDEAAAVALQRPAQLSRYESQKRRDWNTFLQYLRNHRPPLTLARCSGAHVIEFLRYLDQFGKTKVHAAGCAYYGQRAPPGPCPCPLRQAWGSLDALIGRLRAAYEESGRTPESNPLAARAVRIYLREVRDSQAKARGIPYEKKTKRKRAQQQATEPSTSSSDAAAGSGRARAAAAAASAAQAGGSSAAPSST

SEQ ID NO: 100 - protein, 201 aa, Zea mays
MDMPPNPDSPSSGGSNSIGRPSGGGASPSVGSTMPQSSPSRYEAQKRRDWNTFGQYLRNHRPPLSLAQCSGAHVLEFLRYLDQFGKTKVHGPACPFFGHPNPPAPCPCPLRQAWGSLDALVGRLRAAYEENGGRPESNPFAARAVRLYLCEVREHQARARGVSYEKKKRRKPQQLPGPGDSSGLHGHAHQPPPPPPAGAAC

SEQ ID NO: 101 - protein, 219 aa, Glycine max
MDAASEAAAAVAAPQAEGSPAAAAAPSRYESQKRRDWNTFLQYLRNHKPPLTLARCSGAHVIEFLKYLDQFGKTKVHIAGCPYFGHPNPPAPCACPLKQAWGSLDALIGRLRAAFEENGGRPESNPFATRAVRIYLKEVREGQAKARGIPYEKKKRKRTAVTTSVTSAATVMSTLTGSSPNNTITNGAGSTTGNTTNGAGVSVPSVAATATPNVTATAV

SEQ ID NO: 102 - protein, 203 aa, Glycine max
MDSNIQDFIDTCNSDNTCNLITNSITTANLTASAASSSPASTSITSSSRYENQKRRDWNTFGQYLKNHRPPLSLSRCSGAHVLEFLRYLDQFGKTKVHTPICPFYGHPNPPAPCPCPLRQAWGSLDALIGRLRAAFEENGGKPEANPFGARAVRLYLREVRDLQSKARGISYEKKKRKRPPPQQQPMPIPHHPLPPPGASATH

SEQ ID NO: 103 - protein, 164 aa, Glycine max
NRSSDDLRSKRFCRRNFDTTISVVVNHRHLSDVLYDPVFGVIVGLPIVVVTKVHTPICPFYGHLNPLLPCPCPLRQAWGSLDALIGRLRASFEENGGKPEAELFGARLVSLYLCEVRDLQSKAEELTMRRRKGRKRPSPHQQPMPISTPSQLSTSEVPLLSIVL

SEQ ID NO: 104 - protein, 172 aa, Glycine max
MESVASPIVTCSSNNNGSGSNNSTTTTPSRYENQKRRDWNTFCQYLRNQRPPLSMAVCGGAHVLEFLQYLDQFGKTKVHNPTCPFFGLPNPPAPCPCPLRQAWGSLDALIGRLRAAYEENGGRAETNPFGARAVRFYLHDVRDFQAKARGVSYEKKRKRPKQQNIANAAATS

SEQ ID NO: 105 - protein, 150 aa, Glycine max
MSAAVAATAAAAMSVYQHSSSSRQEQHRDHELPLTPQRVCVSPPLSRYESQKRRDWNTFGQYLKNHRPPLTLSRCSGAHVLEFLRYLDQFGKTKVHAETCAYFGNSHPPGPCACPLRQAWGSLDALIGRLRAAFEENGGTPEMNPFGTRA

SEQ ID NO: 106 - protein, 179 aa, Glycine max
MSSSGSHGAEGSSSNIPIEYQQQQQQSPVTLSRYESQKRRDWNTFGQYLKNQRPPVPLSQCNCNQVLDFLRYLDQFGKTKVHLQGCMFYGQPEPPAPCTCPLRQAWGSLDALIGRLRAAYEENGGSPETNPFASGSIRVYLREVRECQAKARGIPYKKKKKTTKGNMEESSTSSSIHFS

SEQ ID NO: 107 - protein, 247 aa, Glycine max
MSAAVAASAAAATMSGYHHNSSSRQQQHIDHELALTPQRVCVSPPLSRYESQKRRDWNTFGQYLKNHRPPLTLSRCSGAHVLEFLRYLDQFGKTKVHAETCGYFGNSHPPGPCACPLRQAWGSLDALIGRLRAAFEENGGAPEMNPFGTRAVRLYLREVRDAQAKARGIAYEKKKRRKPQQSGHDHDHDAMMMVGSTSDVNYSSGYSGGGGGAFVPHQQMLSDSNGTASTSDGVSYFSSSSQCGGFN

SEQ ID NO: 108 - protein, 167 aa, Glycine max
MSTQIQNGSSSSSQQPLSRYESQKRRDWNTFGQYLRNQSPPVPLSQCNFNHVLDFLRYLDQFGKTKVHLHGCIFFGQPTPPAPCACPLRQAWGSLDALIGRLRAAYEEHGGSPETNPFGGGAIRVYLREVKECQAKARGIPYKKKKKKRNPILKGTQRAKDIEQQAS

SEQ ID NO: 109 - protein, 207 aa, Glycine max
MDSIQEFMESCNTDITTTTATTTSNSLVGSSNSPSASSTTSSRYENQKRRDWNTFGQYLKNHRPPLSLSRCSGAHVLEFLRYLDQFGKTKVHTPICPFYGHPNPPAPCPCPLRQAWGSLDALIGRLRAAFEENGGKPETNPFGARAVRLYLREVRELQSKARGISYEKKKRKRPPPPPPPQQQLQQSLPLPLHHHHHHHLPPPGATQ

SEQ ID NO: 110 - protein, 176 aa, Glycine max
METVPTPTTTNHPTTAAAVSSPSSGSNNSGSTTTPSRYENQKRRDWNTFCQYLRNHRPPLSLALCSGAHVLEFLHYLDQFGKTKVHNHPCPFFGLPNPPAPCPCPLRQAWGSLDALIGRLRAAYEENGGRPETNPFGARAVRIYLRDVRDFQAKARGVSYEKKRKRPKPKVTPTPT

SEQ ID NO: 111 - protein, 202 aa, Glycine max
MNSFQEFDSSNTDSNSTKAIINFTSGNSTNFPPAPPPSSSSPPCSSSSSGTTTLSRYENQKRRDWNTFGQYLRNHRPPLSLARCSGAHVLEFLRYLDQFGKTKVHTQLCPFFGHPNPPAACPCPLRQAWGSLDALIGRLRAAFEENGGKPEANPFGARAVRLYLREVRDSQAKARGISYEKKKRKRPQQPPLPPSNNAKVVI

SEQ ID NO: 112 - protein, 235 aa, Glycine max
MDSASGEAPPPPQPTSVEAPPPSGSSAPATSAPAPTQPEGSSPAPLSRYESQKRRDWNTFLQYLQNHRPPLTLARCSGAHVIEFLKYLDQFGKTKVHVTGCPYFGHPNPPAPCTCPLKQAWGSLDALIGRLRAAYEENGGRPESNPFGARAVRTCLREVREGQAKARGIPYEKKKRKRTTMTVSAVSSGGGSSSAVAATSGGGDSGDTIIGGGVGSSASLASTTTATANVTTTTV

SEQ ID NO: 113 - protein, 177 aa, Glycine max
MSSSGSHGTEGSSSNIPIEYQQQQQSPVTLSRYESQKRRDWNTFGQYLKNQRPPVPLSQCNCNHVLDFLRYLDQFGKTKVHLQGCMFYGQPEPPAPCACPLRQAWGSLDALIGRLRAAYEENGGSPETNPFASGSIRVYLREVRECQAKARGIAYKKKKKTTKENAEESSNSSIHFS

SEQ ID NO: 114 - protein, 175 aa, Glycine max
MSSSSSKDFGEGSSSPTDNYQQSPGTLSRYESQKRRDWNTFGQYLKNQRPPVPLSQCNCNHVLDFLRYLDQFGKTKVHLQGCMFYGQPEPPAPCTCPLRQAWGSLDALIGRLRAAYEENGGSPETNPFASGSIRVYLKEVRECQAKARGIPYKKKKKASNQSKGNDESSSTMHFS

SEQ ID NO: 115 - protein, 184 aa, Glycine max
MSSKGKEVAEGSSTTRPSHGGDDDHHHNQQQQLPLSRYESQKRRDWNTFGQYLRNQRPPVALSQCSSNHVLEFLRYLDQFGKTKVHSQGCLFFGQTEPPGPCTCPLRQAWGSLDALIGRLRAAYEENGGLPETNPFASGAIRVYLREVRDSQSKARGIPYKKKKKKRNVIKPNGDTSSSNLPMQ

SEQ ID NO: 116 - protein, 211 aa, Glycine max
MDSIQEFMESCNTDITTTATTTMNTSSNSLVASSNSPSASSTTSSRYENQKRRDWNTFGQYLKNHRPPLSLSRCSGANVLEFLRYLDQFGKTKVHTPICPFYGHPNPPAPCPCPLRQAWGSLDALIGRLRAAFEENGGKPETNPFGARAVRLYLREVRELQSKARGISYEKKKRKRPPPPPPPPPQQQQSLPLPHHHRHLHHHLPPPGATQ

SEQ ID NO: 117 - protein, 181 aa, Glycine max
MSGEMHTDSASSSRAAAASDQRHQQPAAAPLSRYESQKRRDWNTFGQYLKNQTPPVSLSQCNFNHVLEFLRYLDQFGKTKVHLHGCIFFGQPDPPAPCTCPLRQAWGSLDALIGRLRAAYEERGGSPETNPFGSGAIRVYLREVKECQAKARGIPYIKKKKKKNQLKGPHDAPKSFKQLAT

SEQ ID NO: 118 - protein, 194 aa, Glycine max
MSSKGKEIAEGSSTTTTATRSSHGGDDHHHHHNQQQQQQQQQLPLSRYESQKRRDWNTFGQYLRNQRPPVALSQCSSNHVLEFLRYLDQFGKTKVHSQGCLFFGQTEPPGPCTCPLRQAWGSLDALIGRLRAAYEENGGLPETNPFASGTIRVYLREVRDSQAKARGIPYKKKKKKRNVIRPNGDTSSSNLPMQ

SEQ ID NO: 119 - protein, 163 aa, Glycine max
MSSSSGKDLGEGSSSPTENYQQSPGTLSRYESQKRRDWNTFGQYLKNQRPPVPLSQCNCNHVLDFLRYLDQFGKTKNLQHLAHAPFRQAWGSLDALIGRLRAAYEENGGSPETNPFASGSIRVYLKEIRECQAKARGIPYKKKKKASNPSKGNDESSSTMHFS

SEQ ID NO: 120 - protein, 218 aa, Glycine max
MDAASGAAPAALAAPQSEGSPAAPSRYESQKRRDWNTFLQYLRNHKPPLTLARCSGAHVIEFLKYLDQFGKTKVHILGCPYFGHPNPPAPCACPLKQAWGSLDALIGRLRAAFEENGGRPESNPFATRAVRIYLREIREGQAKARGIPYEKKKRKRTIVTTTVTAAATVMSTITGSSPNNTITNGAGSSTGNTTNGAGVSEPSAAATATPNVTTAAAV

SEQ ID NO: 121 - protein, 181 aa, Glycine max
MSGEMHTDSASSSRAAATSDQRHQQPAAAPLSRYESQKRRDWNTFGQYLKNQTPPVSLSQCNFNHVLEFLRYLDQFGKTKVHLHGCIFFGQPDPPAPCTCPLRQAWGSLDALIGRLRAAYEEHGGSAETNPFGSGAIRVYLREVKECQAKARGIPYTKKKKKKNQLKGPHDAPKSFKQLAT

SEQ ID NO: 122 - protein, 229 aa, Glycine max
MASASGEAPPPQPTSTEAAPASGSSAPAISAAAPQPGGSSPAPPSRYESQKRRDWNTFLQYLQNHKPPLTLARCSGAHVIEFLKYLDQFGKTKVHITGCPYYGYPNPPAPCACPLKQAWGSLDALIGRLRAAYEENGGRPESNPFGARAVRIYLREVREGQAKARGIPYEKKKRKRTTVVMVSSGGGGSSGAVASPSGGGDTAIGGGAGSSASLTSSATATANDTTTTV

SEQ ID NO: 123 - protein, 203 aa, Glycine max
MDSNIQDFIDTCNSDNTCNLITNSTTTNLTAASAASSSPASTSITSSSRYENQKRRDWNTFGQYLKNHRPPLSLSRCSGAHVLEFLRYLDQFGKTKVHTPICPFYGHPNPPAPCPCPLRQAWGSLDALIGRLRAAFEENGGKPEANPFGARAVRLYLREVRDLQSKARGISYEKKKRKRPPPQQQPMPIPHHPLPPPGASATH

SEQ ID NO: 124 - protein, 199 aa, Glycine max
MNSFQEFDSSNTNSTKATNFPSSSSAAPAATAPPCSSSSSSTTLSRYENQKRRDWNTFGQYLRNHRPPLSLSRCSGAHVLEFLRYLDQFGKTKVHTQLCPFFGHPNPPAPCPCPLRQAWGSLDALIGRLRAAFEENGGKPEANPFGARAVRLYLREVRDSQAKARGISYEKKKRKRPQQPSLPPSNNAINQLKFLHIPR

SEQ ID NO: 125 - protein, 171 aa, Glycine max
METPTTNPTTAAVSSPSSGSNNSGSTTTPSRYENQKRRDWNTFCQYLRNHRPPLSLSLCSGAHVLEFLHYLDQFGKTKVHNHPCPFFGLPNPPAPCPCPLRQAWGSLDALIGRLRAAYEENGGRPETNPFGARAVRTYLRDVRDFQAKARGVSYEKKRKRPKPKVTHPTPT

SEQ ID NO: 126 - protein, 205 aa, Sorghum bicolor
MDLIPHPDSPHSDNSGGVGGASSAGAVSALASPSRYESQKRRDWNTFGQYLRNHRPPLSLARCSGAHVLEFLRYLDQFGKTKVHTPACPFFGHPAPPAPCPCPLRQAWGSLDALVGRLRAAYEENGGRPENNPFGARAVRLYLREVRDHQSRARGVSYEKKKRKKAPAHPVPAAVISSSSSHDGNGHHHYEHHQMPPPPPPGAAA

SEQ ID NO: 127 - protein, 284 aa, Sorghum bicolor
MSTSGGAGARSWSPPRRASRYESQKRRDWHTFTRYLAAHRPPLELCRCSGAHVLEFLRYLDRFGKTRVHAPLCAAYGGGGGGPALVAAAPCQCPLRQAWGSLDALVGRLRAAFEERHGARGSGTIWTSSQSQSQSQQPAVVDGDAANPFAARAVRLYLRDVRDAQSRARGISYSRKKKKRSKQQDGAAAAAAGCARPHVNGATSLMHAAAPPPAPPPPPPPPPPPPYCLAGVPFEFCDYGTVLGGVTANGAPGFYLPSLFNTFGKLLIIYLLRSKTYYLFISLR

SEQ ID NO: 128 - protein, 215 aa, Sorghum bicolor
MDPSGPGPSSVMGAAGGGEAPAVAPPRPAQLSRYESQKRRDWNTFLQYLRNHRPPLTLARCSGAHVIEFLRYLDQFGKTKVHAAGCAYYGQPAPPGPCPCPLRQAWGSLDALIGRLRAAYEESGGTPESNPFAARAVRIYLREVRDSQAKARGIPYEKKKRKRAQQQQAAAAADPASTSSSAAAAGGSGTSGRAAAAAAASAAQAGGSSAAPSTT

SEQ ID NO: 129 - protein, 320 aa, Sorghum bicolor
MLVMAAKSSVFFLTPRRCRRTNRGGLHADLRREEQADDMQVGGGAADSPGAAAGAEAPRPSRYESQKRRDWHTFGQYLRNHRPPLELARCSGAHVLEFLRYLDQFGKTKVHAPGCPFFGHPSPPAPCPCPLRQAWGSLDALVGRLRAAFEEHGGRPEANPFGARAVRLYLREVRDSQAKARGIAYEKKRRKRPSASSSQSSPQAATTPPQQAPPVSSPALSDVVAERADVRAHVPDAGHQQHHHLHQHQHQHHFFMPHPQFLHGFSLLPGNPEAVAANGNGGGSSSASVAAGNGDEIALAMAAAAEAHAAGCMLPLSVFN

SEQ ID NO: 130 - protein, 258 aa, Sorghum bicolor
MEFAGGGIAAPAADSPGAGASRPSRYESQKRRDWHTFGQYLRNHRPPLELPRCSGAHVLEFLRYLDQFGKTKVHASGCPFFGHPSPPAPCPCPLKQAWGSLDALVGRLRAAFEEHGGRPEANPFGARAVRLYLREVRDSQAKARGIAYEKKRRKRHPAAHRQPKQQQDGHGQHHHPSQAAPGPVAERRLADVAEPPAPHFLIPHAHFLHGHFLAPVTQPIDPAAGGGGGGAGEDIVLAMAAAAEAHAAGFFMPLSVFH

SEQ ID NO: 131 - protein, 208 aa, Sorghum bicolor
MDLSPNPESPGGGGDGGGGGGGAGGSSSGPSSSSAQGGGTPQTPSRYEAQKRRDWNTFGQYLRNHRPPLSLAQCSGAHVLEFLRYLDQFGKTKVHTAACPFFGHPNPPAPCPCPLRQAWGSLDALVGRLRAAFEENGGRPESNPFAARAVRLYLREVREHQARARGVSYEKKKRKKAQPPDHASGSGGQGPHHHHPPPPAPPSAGAAC

SEQ ID NO: 132 - protein, 251 aa, Sorghum bicolor
MDHHHHHHHHHHHHMIPGQEPSAADGAAPDSFFLGPAAAVIFPGGAGASGAGSSSSGAAALGSSVGGGGGPSPSSSSPSLSRYESQKRRDWNTFGQYLRNHRPPLSLSRCSGAHVLEFLKYMDQFGKTKVHTPVCPFYGHPNPPAPCPCPLRQAWGSLDALIGRLRAAYEENGGTPEMNPFGARAVRLYLREVRETQARARGISYEKKKRKKPSAASAAAAGPSSEGSPPPGPSGGGGGPDTSVSPQFIMP

SEQ ID NO: 133 - protein, 199 aa, Sorghum bicolor
MDMSPNPDSPSSGGGNGIGPSSGGASPSVGSMTAPQSPSRYEAQKRRDWNTFGQYLRNHRPPLSLAQCSGAHVLEFLRYLDQFGKTKVHGPACPFFGHPNPPAPCPCPLRQAWGSLDALVGRLRAAFEENGGRPESNPFAARAVRLYLREVREHQARARGVSYEKKKRKKPQQLPGDSSGGLHGHTHQPPPPPPAGAAC

SEQ ID NO: 134 - protein, 325 aa, Sorghum bicolor
MEPGPDAPAGGGGTSSSAPAETGPSSSSAAAAAAASSSSNRQQAAEQEAAPQQQAGAQQPQRQQPAAAPPAQPQAQQPQPLAQQPPPPPPPPAGLSRYESQKRRDWNTFLQYLRNHKPPLTLARCSGAHVIEFLRYLDQFGKTKVHAEGCAYFGQPNPPAPCACPLRQAWGSLDALIGRLRAAYEESGGRPESNPFAAKAVRIYLRDVREAQAKARGIPYEKKKRKRGSAAAPPVAPPPVVTAGTTSGAAGGEEEEDDDDEPSPSAAGERPQQQTTTPASASASAPPPAASTSSASASSSTAATATATVTTTTTRKEEEGSAPSS

SEQ ID NO: 135 - protein, 240 aa, Sorghum bicolor
MDPSGPAAGPSSSAARGGGDALAQAQPQQAAAAQPHQAPPPPQQQLSRYESQKRRDWNTFLQYLRNHRPPLTLARCSGAHVIEFLKYLDQFGKTKVHAAGCAYFGQPNPPAPCPCPLRQAWGSLDALIGRLRAAYEESGHAPESNPFAARAVRIYLRDVRDAQAKARGIPYEKKSRKRKQPAAGSGEASSSSAAAAAREAGAAGDGSGGSAAATKAAPTTGQGSGTTAAAAAAPTSTSRV

SEQ ID NO: 136 - protein, 258 aa, Sorghum bicolor
MQVGGGAADSPGAAAGAEAPRPSRYESQKRRDWHTFGQYLRNHRPPLELARCSGAHVLEFLRYLDQFGKTKVHAPGCPFFGHPSPPAPCPCPLRQAWGSLDALVGRLRAAFEEHGGRPEANPFGARAVRLYLREVRDSQAKARALPTRRSAGSGRPLRGPSRRRRPRRPRRSRLLPSPRRPCRTWCPRGPTCGRTCRTPGTSSTITCTSTSTSITSSCHTRSSCTGSACCRATPKRWLPMATAAAAAALASLPATVTR

SEQ ID NO: 137 - DNA, 25 bp, Artificial sequence
  attB1 site
acaagtttgtacaaaaaagcaggct

SEQ ID NO: 138 - DNA, 25 bp, Artificial sequence
  attB2 site
accactttgtacaagaaagctgggt

SEQ ID NO: 139 - DNA, 54 bp, Artificial sequence
  VC062 primer
ttaaacaagtttgtacaaaaaagcaggctgcaattaaccctcactaaagggaac

SEQ ID NO: 140 - DNA, 53 bp, Artificial sequence
  VC063 primer
ttaaaccactttgtacaagaaagctgggtgcgtaatacgactcactatagggc

SEQ ID NO: 141 - DNA, 3845 bp, Arabidopsis thaliana
gtagcataaataggcaattgcctaaatttgacattctaaggtcaaactttatgatttggtcatgattttattactataaatattttatttatgtaaatgatgaatgtatacaactttccacattttaagtttcatattcataaagaaaaaattggacactaaattttattgttacacattgctacttcttgtattccaaaaagctaaatacaaagtaagtccaagatatctttaattgtatgaaatgcattaaatctaatactactatgatatctccattttcatgacctttttaatcttacacctttttttttcttttttgaaatattgacacaattacaaaaatcatcatcttaccgtgaatacatgatctctctctcaattgaaatttgttttataaaaattattgcgatacgatgtcgttattggattttaaaaaatatgaacgaaaaatgaagagttgcgacttttatgaagagttgtgacttttatgaaaagttgtgagttttccaaaagtttgtgacctttccgataagaaataataagaaccttttcacactacccttcgttttgaatatattgagggatatcctctcattttaggataacggattttcttgacttcttcatctactataaatttaatattctaagtgtactttaatattgttgagtggttcgtttacaccgtggtgttaggtaccaatacatcagtaagtaagatcgttctatcctgggaggatatatttcattaacctcggatacttgagggaaataatatgaattccttaaggcgatactgcacattcagtgggctcgattttcttcgttaagaaaaatattttgtatattatttataatctgtttctggttctattttctatactagcttaattctctagttttgaaaatactctttaaactctgttgttcttatcaagttcttcaaaattttatttttaagtatcttaataatacaaaatgaacaacagacatgacctttatatccgtagattaaaaattagaggggagcctatgctcaactctctttctagttttcatctaatgactgctcgcagaagaaagtataaacaaaaactatacatagttttgcacaaatatacaagtttgagaattcgacataataggcaaaaaatagcttattatatgaactaaatcatttttaattaagaatataagtatagaggattattagttaaacaatatttataatatatatcatcaagctattcaaagttagcttaacatttataacattcaactaactgtctaactaatgttttgtgtcaagaaaaaacaaccaatagggcattggcatctataattagtgtttagaacagacttcttccgtttcatattagttgttcatcttattataaatagttatctcaaattacttattaatttttaaaaatcaagctcgaattcatttatttattttttcattatacccttcttttttatgatttattaggaacttttttcgtgtaaagaaaaactggacaattaataagatagaactcaaagatacatatcgctactagcacaaccatttagccaaaaagatctttatgtgaattttactgtttaatactattcttttaattaaataatttaaaaatgtaattgataagctttatatatagttgaccattttctcttttacgttttattcactttattcaagagatattaattcgaattctgagaacaaaaaaaacttttgatatatatacacttcttcttacgaaattcctataaaatttagtcgtttttatccaaacagtataaaggcggaatcaaaagttaactaaatttttattctttaaattgtacatattctatagtcatttttactaaaaatacagagtttaaactaaagctattcaattcggccaattcgtagccaaaacgctagcttcgcccctagtactaaaaccttgtaatttgcttctttttttttaattatagaatttaaaaactcataaattcaaaatccttttatctttacgcgatgcgaattcgaattcataaacctaataatgaaaacttttttcaagtaaaaaatgtgacctgcagatttgatggtagatacctaacttttgccaaatgtaaagatcaaataataagttgaaaaaatgtgaatattttttttttcttttcatgcagagtctagtccaagaataaaaacaaaaaaagttaggttatttttgtcattttaatcaaggtattgtcaatcactgccacaaaaaaaaaaaaaaaaaagaaggggttcactatagttgaaggaaacaaaatcatattattctcaactttcaaaaaaaatttctttttataactctcaatagtattaagctttttcctgtctacatctatcatttccaatcttactgtcaacctcttttcatttccacaaaagccctaactaggtccctttacaattaccaaaataataccattactacccttaacccccacccccacccccacccccacccccaaaccccatttctaggaaaaaatcagccaatctatgtatacattttcagaaattaaacttcatacaaacatcaaagttttttttaaaattttttttaggaatgatcaagttattgactataaaaccaaagaaagcttatttctttcgtctcgtgacattacatcgagtatgaggaacaaattacctattttttaaaatatgaatctgaatatagagtcttttgagtattttgaaataaaattaaagatagggaaaaagttttgaaaaacactttaacttaatttggctgaaattattattacgatatcaaattttatggagggacttttatcctttcactatttaatagtgtattttaggagtatatatgtgatcacgtgcacacgttactatttataatgatgatcacatggacacacacacatatatacactgttaaataatgtagggggtaaaaggtccttttcagagttcagtattattacagcagtttcgatcaaagttcgaatatatttcaaacattttccctttaaacataactactcatttaataattgatggtacatcaaattcgagaagataaaaactccttttaaaacttgtgatatatttaagcgttagagatgagttttaaaaagaacgattctttccttttcagttaaccttttaattctaactttttacgtgacatatatatttagcaccataagataaataaattaagatttaaaattctgagtcaattaaaactagacaaacaagttactcctccattttaatttgtttgtcatgcattctgataacttttcaatttcagcttttcatatttaaagagtgttctactatacattctacgcatcttttaaaataacaagattcaaaaatattttttatttttttttactcaaaattaagtcaggacaacattaaaacagaaaaaatataatcgaaataattatcttttaaataacaaatcaaaaagaaaaacgaatcacataaattaatccgaacaagataagagaaactaaagttcagccccccctcctctttccccaatcttaacatatcaccccctgtctctctaaattcactctattttattccatcatgtagctaccagacctcacatggtcatcataagcaagcaacataaaaaccctaattcactataaatctattggttcttataacctactccctagctcctctattaccctaaaaaaaacttcttattcatcttatacctcacctctcttctctctctcccaaataaaagatcatcaaatcgaacaaacaaaaaaaaaacccactaagatcaaagata

SEQ ID NO: 142 - DNA, 1368 bp, Lycopersicon esculentum
atggaagcttttcatcatccccctattagctttcactttccctatgcttttcctatcccaacaccaacaaccaattttcttggaactccaaattcatcatcagttaatggaatgatcatcaacacttggatggatagtagaatttggagtagacttccacataggcttattgatagaatcattgcttttctaccaccacctgctttctttagagctagagttgtgtgtaagagattctatggacttatttactctacacattttcttgaattgtacttgcaagtttcacctaagaggaactggttcattttctttaaacaaaaagtaccaagaaacaacatttacaagaacgtgatgaatagtagtaactcaggagtttgttctgttgaaggttacttgtttgatcctgataatctttgttggtataggctttcttttgctttaatcccacaagggttttctcctgtttcatcttctggtggattaatttgctttgtttctgatgaatctggatcaaaaaacattcttttatgtaatccacttgtaggatccataattcccctgcctccaactttaaggcctaggctttttccttctattggtttaactataaccaacacatctattgatatagctgtagctggagatgacttgatatcaccttatgctgttaaaaacttaactacagagtcatttcatattgatggtaatggattttactcaatatggggtacaacttctacacttccaagattatgcagttttgaatcaggcaaaatggtgcatgtacaggggagattttattgcatgaattttagtccttttagtgtgctttcttatgatatagggactaataactggtgcaagattcaagccccgatgcgacgattcctacgttcaccgagccttgttgaagggaatggtaaggttgttttagttgcagcagttgaaaagagtaaactgaatgtgccaagaagtttgaggctttgggcattgcaagattgtggtacaatgtggttggaaatagaaagaatgccacaacaattgtatgtgcagtttgctgaagtggagaatggacaagggtttagttgtgttggacatggtgaatatgtggtgataatgattaagaataattcagataaggcattgttgtttgatttctgtaagaagagatggatttggatacctccttgtccatttttgggaaataatttagactatggtggtgttggtagtagtaataattattgtggagaatttggagttggagggggagagttgcatggatttggttatgaccctagacttgctgcacctattggtgcacttcttgatcagttgacattgccctttcagtcattcaactga

SEQ ID NO: 143 - DNA, 561 bp, Agrobacterium tumefaciens
ctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgg

SEQ ID NO: 144 - DNA, 1240 bp, Sesbania bispinosa
atccctcactcactgtcattttcaacctatttccatttccacaaaaaccctaaagtccaaaccagtgcttctttctcaattaagcccctcttggtctttggctctgggataggtcaaagcaacccagcaagcaacttccctttttccttaaaataaaaaacagacactgtccattcattttcattcattcctaaaaatggtttcaagaaaacagggttagcaaaaagcactcctagcaattcaagtattctaggagttatcaaaaaccgaagcaattgataggatcttccttttcttcactctccataactgttcatcatcatcaaaaaaaccaagcttgataatcagatcaaagatccaatctttctgattagtgagtgagtgatgagttcctttcaagaatttgactcatcatcaaacactacagatgacagcaaaggcatcatcaatttcacacctgcaatgaacagtaacaatttcacatcttcagcagcctcatcatcacaaccaccaactctgagtcggtatgagaatcaaaagcggagagattggaacacgtttgggcagtatcttagaaaccacagaccaccactgtccctgtctcgctgcagtggagctcatgtgcttgaattcttaaggtaccttgaccagtttgggaaaactaaggttcacacacaggtttgtcccttctttggacacccaaacccacctgcaccatgtccttgtcctctacgccaagcttggggaagccttgatgctctcataggtcgacttagagctgcttttgaagagaatggagggaaaccagaaacgaacccttttggtgctcgtgctgttaggctttaccttcgtgacgtgcgtgattcacaatccaaagctagaggagttagttatgagaaaaagaagcgaaagcgtccccaacaatctcaacctccaaatgcaacttagtaaggccacaaaactgcataagttgtgatttgacattggatcactgcaatatggggacaccctttgggattttattacaaaatgaatgtgtaacctaagaagtactgctcttattaatggatatatcaagtaatttaagcattaattagtatgtagtgtagtagcagtaaaaagactgtgaagtgagaggctgcagtgcagtgcaacacatatcgctcaatttgtaataatcaaataaattatatatatttatgtatgtattttggttttaaaaaaaaaaaaaaaaaaaaaa

SEQ ID NO: 145 - protein, 187 aa, Sesbania bispinosa
MSSFQEFDSSSNTTDDSKGIINFTPAMNSNNFTSSAASSSQPPTLSRYENQKRRDWNTFGQYLRNHRPPLSLSRCSGAHVLEFLRYLDQFGKTKVHTQVCPFFGHPNPPAPCPCPLRQAWGSLDALIGRLRAAFEENGGKPETNPFGARAVRLYLRDVRDSQSKARGVSYEKKKRKRPQQSQPPNAT

SEQ ID NO: 146 - DNA, 1433 bp, Sesbania bispinosa
aaaaaatggtttcaagaaaacagggttagcaaaaagcacacctagccattctacgctttcaagtattctaggagttatcaaaaaccgaagcaattgatagggtactctctccaactgttcttttaccctcctctccctttaatttcctgttatgttctctctcttgaatctctgctcaaatctgatcaacaaacccatttattttccttagattagatcttctttttcttcactctccataactgttcatcatatcataaaaaaaaaaaaccaagcttgatatataatcagatcgaagatccaatctttctgattagtgagtgagtgagtgagtgatgagttcctttcaagaatttgagacctcatcaaacactacagatgacaacaaaggcatcatcaatttcacacctgcaatgaacagtaacaatttcacatcttcatcatcctcttcaccacaaccaccaactctgagtcggtatgagaatcaaaagcggagggattggaacacgtttgggcagtacctaagaaaccacagaccaccactgtctctgtctcgctgcagtggagctcatgtgcttgaattcctaaagtaccttgaccagtttgggaaaactaaggttcacatacagctttgtcccttctttggacacccaaacccacctgcaccatgtccttgtcctctacgccaagcttggggaagccttgatgctctcataggtcgacttagagctgcttttgaagagaatggagggaaaccagaagctaacccttttggtgctcgtgctgttaggctttaccttcgtgaagtgcgtgattcacaagccaaagctagaggagttagttacgacaaaaagaagcgaaagcgcccccaacaatcacaacaacctcaacctccaaatgcaacttaattagtaaggccacaaaactgcataagttgtgatttgacattggatcactgcaatatggggacaccctttgggattttattagaaaatgaatgtgtatcctaaacagaagtactgctcatcttatatattaatggatatatcaagtaatttaagcattaattagtatgtagtgtagtagcagtaaaaagactgtgaagtgagaggcctgcagtgcaacagaccaatacataccgctcaatttgtaataatcaaataaattttatatatatttatgtattttggtttatggtatcctctgttactagctccaggtttagccataattaagactctttatctttatcaagagtgtgttaagtgggatgaaaccagagagttttttcataattccagtttgtgttgtcttccttatgtatatacttaaagatgttatatgacaaaataatggatttcaaggtcaacatgtttccatatcctccttggatatatggggcagtttcttttaaaaaaaaaaaaaaaaaaaaa

SEQ ID NO: 147 - protein, 190 aa, Sesbania bispinosa
MSSFQEFETSSNTTDDNKGIINFTPAMNSNNFTSSSSSSPQPPTLSRYENQKRRDWNTFGQYLRNHRPPLSLSRCSGAHVLEFLKYLDQFGKTKVHIQLCPFFGHPNPPAPCPCPLRQAWGSLDALIGRLRAAFEENGGKPEANPFGARAVRLYLREVRDSQAKARGVSYDKKKRKRPQQSQQPQPPNAT

SEQ ID NO: 148 - DNA, 1359 bp, Sesbania bispinosa
atcttccttcaaacttgctttcaagtttcaaaccatctctctctttctctctctctctcttgaagcacagtttaatcctttgctcaaatctgatccaagtgtttttccttttggttctattcattcccatcctcctcctcagatttaatttcttgaagattgaagatccaatctagatcagaaacaacaaccatgaattctcttcaagatcaatttgaaccctcctcatcaaacaaaggcataaaccccatcacaaacatagcagggattaacagctccaatttaacaacaacaaccatgatgaccatgtcctcctcatcatcaacatcatccacatcaccaccatcaacttctccaagtaggtatgagaatcaaaagaggagggactggaacacttttgggcagtacctaaggaaccacagacctccactttccctctctcgctgcagtggagctcatgtccttgagttcctaaggtatcccgatcagtttgggaaaacaaaggttcacacacagctttgtcctttctttggacacccaaacccacctgcaccatgtccatgtcccctgcgtcaagcatggggaagccttgatgcactcataggtagactcagagctgcttttgaagagaatggagggaaaccagaggctaacccttttggtgctcgtgctgttaggctctatcttcgtgaagtgcgtgattcacaagctaaagctagaggtatcagctatgagaagaagaagcgcaagcgtccccctcatcctctcccaccaccacctgcattgccaccaacaactcatgaatagtgtgtaggttgtgatttggcatggggaaactgcaatgtaagatatgcactgtggagggaactgcagtattactggtagaattgtagtagcagtgataaatgaaatgagatgagagaaagatgaagtagctagtggtggtgatagtatgagaagtaaaaaggggatgtgaggagaagagggttgtgtgtgactgtgacctataattcaccgtccaaatttgtaataaatataagtgtgtacttgaaggtgggtcatttatgagaagagtaatggatttgcagatgatgttccatgtcttcattggatgcggtaaaatgcaattactttacttgtgtttattttttaccccctttgttgtgtttagcatcatattatattatatatatttttgtgtttgggttttcactcagatagcaggggggccattctggtagctttatttgccctgtcgaaagttgaattcagggatttcatttcatgtagttttcattcatcataaatatataaactgtagatactgagactggaaaaaaaaaaaaaaaaaaaaaaaaa

SEQ ID NO: 149 - protein, 204 aa, Sesbania bispinosa
MNSLQDQFEPSSSNKGINPITNIAGINSSNLTTTTMMTMSSSSSTSSTSPPSTSPSRYENQKRRDWNTFGQYLRNHRPPLSLSRCSGAHVLEFLRYPDQFGKTKVHTQLCPFFGHPNPPAPCPCPLRQAWGSLDALIGRLRAAFEENGGKPEANPFGARAVRLYLREVRDSQAKARGISYEKKKRKRPPHPLPPPPALPPTTHE

SEQ ID NO: 150 - DNA, 1512 bp, Sesbania bispinosa
taaacagcacaacacagagaagaggggaagaatattaacatgaccattaacaagtgatgatgttaagtttgagtttgcttcacccttttaagaaaaaaaacaatcccctcaccaatctcagaaatcaaagctactaaaaaaacaccaaacactgccacacagctataactagccccttgggagagagcttttctagtattgctcaataaatacttacaacaaacagcaaagtggaacagaccacacaaactcccatcaatctcatcacattctcaacatcaaataatcttcttttgcttcacaccatttgcaggaaccctagaaattaaagagacacaaagtcaaaaccaaccaaccttggatctgcaaaaagcataagggattattaatttccatggattcaattcaagaatttatagacacatgtaactctgacaacaacaagaccatcatcaccaccaccaccacaaccaccttagctactgctacagccacctcttccaccaccaccagcagccggtatgagaaccagaaacgccgtgactggaatacctttggtcaatacctcaagaatcacagaccccctctttctctctctagatgcagtggtgcacatgtccttgaatttctcaggtacttggatcaatttgggaagacgaaagtgcacacaccgatttgtcctttttatggacaccctaaccctccagctccatgtccatgtcctctaagacaagcatggggaagccttgatgctctgataggtcgtcttcgggcagcttttgaggaaaatggagggaaaccagaagctaatccatttggagctagagctgtgaggctttaccttcgtgaggttcgtgatcttcaatcaaaagcaagaggaattagttatgagaaaaagaagaggaagcgtccaccacagcaacaatctatccctctaccaccttcaggtgcaactcattaggaagagaaaacaacaacaaaaatcaaaatgaaaaatgaaaaatgaaaaattctcactctctcttcttagttatctcaatgtttaacttttagtatgatataatttagggtttttttttttttaattttgtgtgttaatttatgtgactgaaagctgctatgtggtacatgtagactgttttgtctttggatcctgttagctgtggcaggagcaaaagtttttaaatatataatatataaaaaaaagaaagaaagatatctgggatatcatgcatctgcttttaaagcagaagacagtgctgtactggtagtagactgatagtgggctgaaaagtgctactactgagagtgaatatacatggactccatcaccaaatcaaaagaaagctacctctaccatggattcttacttctctttctctatatatatgtgtacttccatatatatatgtcaagctataagcttagacttgcagattatgtttgtatatgtatgtctttaatcatactgagctctcacttttggaatataaaaaaaaaaaaaaaaaaaaa

SEQ ID NO: 151 - protein, 188 aa, Sesbania bispinosa
MDSIQEFIDTCNSDNNKTIITTTTTTTLATATATSSTTTSSRYENQKRRDWNTFGQYLKNHRPPLSLSRCSGAHVLEFLRYLDQFGKTKVHTPICPFYGHPNPPAPCPCPLRQAWGSLDALIGRLRAAFEENGGKPEANPFGARAVRLYLREVRDLQSKARGISYEKKKRKRPPQQQSIPLPPSGATH

SEQ ID NO: 152 - DNA, 1016 bp, Amaranthus hypochondriacus
ataacttttttttctctccctctcttccttccaaacacaaccctaattttaatattaaacacccaaaaaaaaatcataaaaaattctctcaaaaattttttatagagaaacatggataatcatcatcatcatcatcatcaaattccttcatcaatgatcctattttcttcatcaactaacactagtaataataataatataacaacttcatcatcatcttcttctccttcaccttcttccccttcgggttcgggttccgggtcgggtccaactactcctacaacttctagtaggtacgagaatcaaaaaagaagggattggaacaccttcggacaatacctaaaaaatcaccgcccaccacttgctcttaaccgttgtagtggagcccatgtccttgaattcctccgttatttagaccagtttggaaaaactaaggtccatacccatctttgtcccttttacgggcacccgaacccgcctgcaccatgcccatgcccgcttcgtcaagcgtggggatctcttgatgctttgatcggacggttaagagctgctttcgaggaaaatgggggtaaacccgaaatgaacccgtttggggcccgtgctgttaggctttaccttagggaagttagagattctcagtctaaagctagagggattagctatgagaagaagaaacgtaaacggatcccaccaaataataataatccgggttcaaatccgggtacgaataatcagatcgggtcgatctcaatgttaccaccatcaggtgctagctgatgatgatgatgatgatgatgatcagtaagatatttaagctatgtgtgtgaatagatcatgtttcttttattatgatgatcatgtgatggttttcttaggtttatattctagggttaagtttattagcaagtagaagtattatttaagttaaggtgttatttatgctattgtcactttctaattatgttaaaagaaaatgaaatgaaattttttttaacaaaaaaaaaaaaaaaaaaaaa

SEQ ID NO: 153 - protein, 221 aa, Amaranthus hypochondriacus
MDNHHHHHHQIPSSMILFSSSTNTSNNNNITTSSSSSSPSPSSPSGSGSGSGPTTPTTSSRYENQKRRDWNTFGQYLKNHRPPLALNRCSGAHVLEFLRYLDQFGKTKVHTHLCPFYGHPNPPAPCPCPLRQAWGSLDALIGRLRAAFEENGGKPEMNPFGARAVRLYLREVRDSQSKARGISYEKKKRKRIPPNNNNPGSNPGTNNQIGSISMLPPSGAS

SEQ ID NO: 154 - DNA, 1458 bp, Artemisia tridentata
gaaacatattttgtgaagtccacttcttttttcaaccatcatacatcatctcaaagctcactgtcaacctcttttcacttccacaaaagccctaatttcaaaccttttttttggacaatttcacatcacttatcacacaccatcctacttctctctctatagtactttgtaggtgtatttgtatacccttttctaggttacaaaatataaagtaacctctcatttttgccaatttccttctattttatagttagatcttccattcttgtgtagcttctttcaagctcagaacttcactcatcacaaatcttgatctacacccacatgtcatcttcctcatccttatattttaaccatttcaagaatcttgatgaaaccctagaaaacccatagttagggtttcttgaatatcttcttctttttagtgaaaccctagatttgattcaaccagatctcactttttagcactgaattatacaccaaaatggaaaaataccactcttttaccatggatgttgttccacaagtagcacaaggagttgactcaatcatcacaacaaaccttacctcaaatatgtcaagtccaacatcatcttcaatatcttcaccctcaactctcagccgctatgagaatcaaaagcgccgtgattggaacacttttgggcagtatctccgtaaccatcgaccacccttaaacctagctagatgtagtggtgctcatgtgcttgagtttttaaggtatctagatcagtttggaaaaacaaaagtccacactcaactatgtccattttttggtctcccgaaccctccagcgccctgcccctgcccgctaaagcaggcttggggcagccttgatgcgcttattggcaggcttcgggcagcttatgaagaaaatggagggaagcctgaaaataacccttttggagctagggctgttaggctttatttacgtgaagttagggattctcaagctaaagctagaggaattagttacgagaaaaagaagcgaaagaggccgccacagtcgcaaacaccgcaatcgccacagccgcctcctctttcttgagcaagctagaggtgtttgtgtggagaatgcttattgaagctgcttatctttagcttattcaagctagttattggtctttattgttaactgagataacaagtaagtagcgttgagagtcgatattatatgataaactgattatctaattatttgtgaacggaatgaacagtttcgagtaaatagcttattcgacactgttgattgagtttacccacagttgatctgataatcagtttggataaacaatatcaaacatttactctcaaaattacttaatttgttgtatcaataacaaagtcacaaaaagtaaagtattatttctaccctagtgataatgggtattattttaactatatatcttagtttaaaatgtctaaaaaaaaaaaaaaaaa

SEQ ID NO: 155 - protein, 193 aa, Artemisia tridentata
MEKYHSFTMDVVPQVAQGVDSIITTNLTSNMSSPTSSSISSPSTLSRYENQKRRDWNTFGQYLRNHRPPLNLARCSGAHVLEFLRYLDQFGKTKVHTQLCPFFGLPNPPAPCPCPLKQAWGSLDALIGRLRAAYEENGGKPENNPFGARAVRLYLREVRDSQAKARGISYEKKKRKRPPQSQTPQSPQPPPLS

SEQ ID NO: 156 - DNA, 1103 bp, Artemisia tridentata
gatcaaagaaaccctagaagtttcatagaagaggaacacaaagaaaccttaaaaacgacaaccatatcttatcatagatcttcttgtaagaacccaaattgggaaaacccttattccttcttataaaatccaagaaaacccatatatattttgttacaaaaaatggatctctaccctgaaatggagtgtaacaattcagaaacctgtcatcgtaacatcgggagtactttagtcactagtggacctgtttcttcatctagttcaccttcaactacttccacacctagccgctacgagaaccaaaagcgtcgagactggagcaccttcggtcagtacttgaagaaccaccgtccaccactctcactttctcggtgcagcggagctcatgttcttgagtttctccgctacctcgaccaatttggaaagactaaggttcatgcgcccatatgtccgttttacgggcatccaaatccgcctgccccttgtccttgtcctctaaggcaagcttggggcagtcttgatgccctgataggccgactcagagcggcctatgaagaaaacggatggcctacagaaacgaacccttttggggctcgtgctgtgaggctctaccttcgcgaggttcgcgatctacaatccaaagcaagaggaatcagctacgagaaaaagaaacgaaagagaggtccaacacaagaacatcaacacttcacaccaaacttttcatctttccaagctttcactcttccaccaggtgatcatcatcaacaacatgtctaaatttcaaaagaaaaaaaggtaatacaagaatcaaagttggatcttgttgttaaccttaatctccttttttatgttctcccatgtgggttagttatcaagattattagtttcatgttgttgctctaagtctagttttagttttttttttttttttttaaaactttttttatgttttttctttttgtagcccagatgttaaccgatctatgaccactcatttagagacatggacactgcgcatgtacttcgcgtatgtgaagttgaattgtagaaataaacccaagcttaatgttaatgtgtggttttattaaaaaaaaaaaaaaaaa

SEQ ID NO: 157 - protein, 204 aa, Artemisia tridentata
MDLYPEMECNNSETCHRNIGSTLVTSGPVSSSSSPSTTSTPSRYENQKRRDWSTFGQYLKNHRPPLSLSRCSGAHVLEFLRYLDQFGKTKVHAPICPFYGHPNPPAPCPCPLRQAWGSLDALIGRLRAAYEENGWPTETNPFGARAVRLYLREVRDLQSKARGISYEKKKRKRGPTQEHQHFTPNFSSFQAFTLPPGDHHQQHV

SEQ ID NO: 158 - DNA, 805 bp, Artemisia tridentata
gaacctctcatttttgccaatttccttctattttatagttagatcttccattcttctgtagcttctttcaagctcagaacttcactcatcacaaatcttgatctacacccacatgtcatcttcctcatccttatattttaaccatttcaagaatcttgatgaaaccctagaaaacccatagttagggtttcttgaatatcttctttttagtgaaaccctagatttgattcaaccagatctcacttttttagcactgaattatacaccaaaatggaaaaataccactcttttaccatggatgttgttccacaagtagcacaagaagttaactcaatcatcacaacaaaccttacctcaaatatgtcaagtccaacatcatcttcaatatcttcaccctcaactctcagccgctatgagaatcaaaagcgccgtgattggaacacttttgggcagtatctccgtaaccatcgaccacccttaaacctagctagatgtagtggtgctcgtgtgcttgagtttttaaggtatctagatcagtttggaaaaacaaaagcccacactcaactatgtccattttttggtctcccgaaccctccagcgccctgcccccgcccgctaaagcaggcttggggcagccttgatgcgcttattggcaggcttcgggcagcttatgaagaaaatggagggaagcctgaaaataacccttttggagctagggctgttgggctttatttacgtgaagttagggattctcaagctaaagctagaggaattagttactaaaaaaaaaaaaaaaaaaaaaaaa

SEQ ID NO: 159 - protein, 170 aa, Artemisia tridentata
MEKYHSFTMDVVPQVAQEVNSIITTNLTSNMSSPTSSSISSPSTLSRYENQKRRDWNTFGQYLRNHRPPLNLARCSGARVLEFLRYLDQFGKTKAHTQLCPFFGLPNPPAPCPRPLKQAWGSLDALIGRLRAAYEENGGKPENNPFGARAVGLYLREVRDSQAKARGISY

SEQ ID NO: 160 - DNA, 921 bp, Artemisia tridentata
gacgccacaaccctctttccacaatcaatcaatcaaattcaatccaaaatccaaaagcacaaaacactcgatcttggttattatggatttagcatcaggatctggcgggccttctggtccacctggtccgccaaatcctgacgatcaggtggaactagctgcagggaaatcaagaactctagcagctgttccacctctaagtagatacgagtcacaaaaacgccgtgactggaatactttcttacaatatttaaagaatcacaagccaccgctgacgcttaccaggtgcagcggggctcacgtgattgagttcctaaagtacctagaccagttcgggaagaccaaagtacacgttacaggttgtccatactttggcttcccgaaccctccagcgccctgtgcttgctcacttaagcaagcatgggggagccttgacgcgctcatcgggcgcctacgggctgcctacgaagagaacggtgggcagcccgaatcgaacccttttggtgcaagagccgtgaggatatatttaagggaagttaaagatagtcaagcaaaagctagaggtattccctatgagaagaaaaaaagaaaaagaagtggtggtgatggtagtacgtccaccgctatcgtggtggcagcaaatacgacgtcggtggcggatgatggtggtggtagtggtggtggagagggtgggactagtggcattatttccaacagtgagccttctcctgttactcccccattatagtaatattattcataaataaaattttatattatatattgagaattatttgtaggcaattttcgaatcatttaattactgtagaacgttagtttattgaatatgacaagacataataattggtaaaaaaaaaaaaaaaaaaaaaaaaaaaaaaaaaaaaaaaaaaaaaaaaaaaa

SEQ ID NO: 161 - protein, 221 aa, Artemisia tridentata
MDLASGSGGPSGPPGPPNPDDQVELAAGKSRTLAAVPPLSRYESQKRRDWNTFLQYLKNHKPPLTLTRCSGAHVIEFLKYLDQFGKTKVHVTGCPYFGFPNPPAPCACSLKQAWGSLDALIGRLRAAYEENGGQPESNPFGARAVRIYLREVKDSQAKARGIPYEKKKRKRSGGDGSTSTAIVVAANTTSVADDGGGSGGGEGGTSGIISNSEPSPVTPPL

SEQ ID NO: 162 - DNA, 813 bp, Artemisia tridentata
ggaaaagacaaagagacagacatcatgtcctagatcttccagaaaggctcaaaaaatttacccaaattaagatcatttttcatataccaaaagtggaaagcagaagaaaaccctaaattgcaatggatccatactcagaagaaagctatgacaacaaccaccaagatttcaacaacctaaccatcatggcatctggctcctcctccacatctcctgcggtggcaaccacccctagccgctacgaaggccagaaacgtcgtgattggaacaccttttgtcagttcctaagaaaccattatccaccacttaatgtttctcaatgtactggaacaaatgtgcttgaattcttaagttacctagaccaatttggaaagactaaggttcataccttggtatgcccattttatgggcatccaaatccgcctgccacttgttcttgtccacttcgacaagcttggggcagtctggatgccctgataggccgtcttagggcggcctatgaagagaatggtggagctcctgagacgaacccttttggggttcatgctgtgaagctttaccttcgtgaggtccgtgatttgcaatcaaaagcaagagggataagctacgagaagaagaaacggaaaaaggctggcaccacaacaagttgataacacttgaattgaaggaacaaaaacgatgcaagttattatagtgatgctggatcttggtatgcattgatcttccggcctactgttattttctttgactttatgctctaatgtggaattatccagactctagggtactatttccgaaaaaaaaaaaaaaaaa

SEQ ID NO: 163 - protein, 175 aa, Artemisia tridentata
MDPYSEESYDNNHQDFNNLTIMASGSSSTSPAVATTPSRYEGQKRRDWNTFCQFLRNHYPPLNVSQCTGTNVLEFLSYLDQFGKTKVHTLVCPFYGHPNPPATCSCPLRQAWGSLDALIGRLRAAYEENGGAPETNPFGVHAVKLYLREVRDLQSKARGISYEKKKRKKAGTTTS

SEQ ID NO: 164 - DNA, 867 bp, Artemisia tridentata
gattcgtaaccctattatccaatttctaaacacacacttcatttgttcgcattatttttaatggatttagcatccggatccgggtcgacaactgacccggatcctaatcctaatcctaatcctgaactacccgtagttcctcctcccgaccctttatcggtttcagtgcctcatccacctagcagatacgaggcacaaaaaagacgtgactggaacacgttcttacaatacctaagcaaccataaacccccattaacattatctaggtgtagtggagcacacgttatcgagttcttaagatacctagaccgatttggaaaaactaaagtgcatgtgactgagtgtccatattttggacaccctaacccacctgcgccttgtgcttgtcagctcaagcaggcgtgggggagtttggacgcgctcatagggcgactcagagctgcctacgacgaaaacggtgggcagcctgagtcgaacccttttggagcgcgtgctgttaggatttatttaagggaagtgagggagagtcaagctaaagctagaggtgttccgtatgaaaagaagaggaaacggcctagtggtgctggtacgatggctatagcgaaagtatcggtggatgacggtcatggtggtggtggtgatgacatttctcgtactactgctccggctacttccacggtttaattaattgtttatatattgttcttgactattgctatttttttaattgtgatagaatattatacattcactagctattactcgtatgaattaattagctaatttatgttatgcattgttttatttaagagtttcttagacgtttaagtaaaagattgaatgttcaccaaaaaaaaaaaaaaaaaa

SEQ ID NO: 165 - protein, 207 aa, Artemisia tridentata
MDLASGSGSTTDPDPNPNPNPELPVVPPPDPLSVSVPHPPSRYEAQKRRDWNTFLQYLSNHKPPLTLSRCSGAHVIEFLRYLDRFGKTKVHVTECPYFGHPNPPAPCACQLKQAWGSLDALIGRLRAAYDENGGQPESNPFGARAVRIYLREVRESQAKARGVPYEKKRKRPSGAGTMAIAKVSVDDGHGGGGDDISRTTAPATSTV

SEQ ID NO: 166 - DNA, 999 bp, Lamium amplexicaule
tttttctcacaccttctctctctacccctcctttcttttccaattaccaaaaaaacccatgaatcccaccgataatcagggctccgccagcagcaacccagcgccgccgtctccggcgcctctgagccggtacgagtcccaaaagaggagggattggaacactttcggacagtacttaaaaaatcagaggcctccggtttcattatcaaactgcaattgcaaccacgtgcttgatttcttgcggtatttggaccagttcgggaaaactaaggttcacttgcatggttgtgttttcttcgggcagcctgaccctcctgccccgtgcacctgcccgctgcggcaggcgtggggtagcctcgacgcgctgatcgggcgattgagggcagcgtacgaagagcacggagggtcacaggagacgaatccttttgggaatggagctattcgtgtttaccttagggaagttaaggattgccaagctaaagcgagagggattccgtacaagaagaagaaaaagaagaggaaaattagtcatcaaatcaatggccatgatgagttgatcaagaatcccaaacagatcaattgaaaaaataaaaaaataataaggttaaactatgcaagttgtagtggatggagcaaaaaggcgtgaactaattaccagaatttatccatcacagcttgagagacccactttaattaggcatacaatagactgtaagatgaaagtctaggcatataattatatatgtaaaacagtaatagatgtagattatatatatacgattatatattatagcaattatatattattaattgcatctttgttgtagtaaatgaagacccctcctccattgagaactttgttgtagagattggagtttgatgaactcattactccaatatatctctcttaatgaaggaatggtcaatgaacatgacttaataattttgtagtttggattctcccataaaaaaaaaaaaaaaaaaaaaaaaaaaaaaaaa

SEQ ID NO: 167 - protein, 174 aa, Lamium amplexicaule
MNPTDNQGSASSNPAPPSPAPLSRYESQKRRDWNTFGQYLKNQRPPVSLSNCNCNHVLDFLRYLDQFGKTKVHLHGCVFFGQPDPPAPCTCPLRQAWGSLDALIGRLRAAYEEHGGSQETNPFGNGAIRVYLREVKDCQAKARGIPYKKKKKKRKISHQINGHDELIKNPKQIN

SEQ ID NO: 168 - DNA, 873 bp, Lamium amplexicaule
atttagatgcaaaaaaaatttaatttcatatggtgtgataaatttatattcctcttgatttagatggagagagggaaagatttagccgaagggtcgtcgtggggcggcgatgtggcggcggctgccaccactccgagccggtacgagtctcagaagcggcgggattggaacacgttcgggcagtttctccggaaccaacggccgccggtggggctgcccgagtgcaacagcaaccacgtgctggatttcatgaggtacttggaccaatttgggaagaccaaagtccacttgcaagggtgcatcttctacgggcagccggagccgcctgccccgtgcacgtgcccgttgaggcaggcgtggggcagccttgatgcgttgatagggcggctccgggcagcctacgaggagaacggtgggtcccctgagacgaacccgtttgctagcgggtcgataaggatttatttgagggaagtgaaggagtgtcaagctaaagctagagggattccgtacaagaagaagaagaaagtaggaggaggagggaagggtgatgatcaatccaattcttcttcttctatgccattttcttgatcaaattaatatgtggccgggattccgtgattagaggaagaaaactacatgtgaagcaggtaattaaatctggaattcttccttaaaactatatatagactcagataactcatcatgcttgggtctcttgctcctatttttcatgttttgcagcgtttgcaaatttcttaagcccttgtaagatcgtaaactttggttttactccaattaatatattcatgtaagtttatgtcatgttcgtttcttaaaaaaaaaaattatggcaaaaaaaaaaaaaaaaaaaaa

SEQ ID NO: 169 - protein, 174 aa, Lamium amplexicaule
MERGKDLAEGSSWGGDVAAAATTPSRYESQKRRDWNTFGQFLRNQRPPVGLPECNSNHVLDFMRYLDQFGKTKVHLQGCIFYGQPEPPAPCTCPLRQAWGSLDALIGRLRAAYEENGGSPETNPFASGSIRIYLREVKECQAKARGIPYKKKKKVGGGGKGDDQSNSSSSMPFS

SEQ ID NO: 170 - DNA, 1300 bp, Peperomia caperata
atccatggccagccatgagagagggaaggacccaattgcgggaacatcttcccggacgagctccggcgagacccatctcgctccggccggccaacttagcaggtacgaatcacaaaagaagagggattggaacacatttggtcagtacttgaggaatcagaggccaccagtgtctattccccagtgcaattgcaaccatgtgttggattttttaaggtacttggatcagtttggcaagaccaaggtgcacctcaacgggtgtgtcttttttgggcaaccagacccacctgccccttgcacttgccctctccggcaggcctggggaagccttgatgcgctcataggccgccttcgcgccgcctatgaggagaacggtggctcgccggagacaaacccttttggagccggaccgattagggtttatcttagagaggtgaaagagtgtcaatcaaaggcaaggggggttccttacaagaagaagaagaagaagagaagccaaactagtaaggaagatcaagatgaagagagctagagcttgatgaaatgtttgggttttggacaagatgaatttcagattcgcatgtcaaggtttgaaaagatgggagtcttaggtttgaaaagatgggagtcttccaaagagctacaaagatatgaatattgtaatgagacatgatattggtgtctaattaagctatgctattaaaatatttcatgattatatttaatttgttgagaatgttttagccctttgggtatatatatttaattcccatctttcttcaaaaaaaaaaaaaaaaaaaaaaaaaaaaaaaaaaaaaaaaaaaaaaaaaaaaaaaaaaaaaaaaaaaaaaaaggaaaggctgctctccctcacagcttgcattggcatgggttcaccagcaaggagcggatgtgtgccctatcccaggaacaacaaagattgagaacttgaaccagaacattggagctctatctgtgcatctcacaccggaggaaatggcggaactggaatcatgtgctaatgcagtccagggtggcagatatccgcccggcgtgactgcttcctggaggaaccccgacacaccacctctctgtcttcccggaaaggacagtgaaaactgagaaccatgctggaaagcaccgcatgtttgagttctatatatgttactgttgttgactttatattcctaagagaaaggtttgctttcggcaaaaacactgcatatgtactctggtgcctatgtacccaacatgagtaaagcaataaactgcgtctgcatcaaaattttacagttgttga

SEQ ID NO: 171 - protein, 175 aa, Peperomia caperata
MASHERGKDPIAGTSSRTSSGETHLAPAGQLSRYESQKKRDWNTFGQYLRNQRPPVSIPQCNCNHVLDFLRYLDQFGKTKVHLNGCVFFGQPDPPAPCTCPLRQAWGSLDALIGRLRAAYEENGGSPETNPFGAGPIRVYLREVKECQSKARGVPYKKKKKKRSQTSKEDQDEES

SEQ ID NO: 172 - DNA, 1502 bp, Eschscholzia californica
atatcaaagctttcaagctagcttgttgtctagtatgtttctcttatcatccaagaaaccctaagaagaataagactaccctcatcacatcttaatcctcaccaaacttcaaccctaaaatccctctctctctctctctcatctatagaagttagataccatgtgaacacataagatcaaagctttttgtgagatttaagaagtcttccaaacaagaaagaagtttgatctcttcaagtaaacagtgcatctaagaatcacctggtctctctctctctctctctatatctatatggtattgtctcttatggagatcatttaagaagcacctgcaagtagaagaaatctagcatctatatcttctttcttgaagctaatgaaatggaagaaacccaagaagattgatatgtcaagactcttcaacttcatcacactataaaagaggagattgcactgttacagattccaagaaggtaaaaagatacagatcactcacaaactagaggaactagctagggttaaactatagagacaagaaagatcatcatcatcatttcatcttcatcagcaccacaattccatggaaagaacaattgttgcatttggagctgaaagctcaagctcagagaataccaacatgagtactactaacaactcagctaattcatctgctgtagcaacttcatcgtcctctcctccaaccctgagtcgatacgaaagccagaaacgtcgagactggaacactttcggacaatacctaaaaaaccacagacctccactgtctctctccaggtgcagcagtgcccatgtcctcgagttccttcgctaccttgatcagtttggaaaaaccaaagtccacgcacaaatctgccctttcttcggtcacccgaacccaccagctccatgcccatgccctcttcgtcaagcttggggaagcctcgatgccctgattggacgtctccgtgctgcttttgaagagaacggtggaaagcccgaagctaacccatttggtgctcgagcaattaggctttacttacgcgaagttcgtgatttacaatcgaaagcaagagggattagctacgagaagaagaaacgaaagcgtcctcctaatcaattaattagtacttcatcaactcaattgccaccttcatcaagtactaatggtgcaactacttaatgtgtatgatcatctaatgaagatatgggtaatggagatctctattctgggttaattactcaaacttgttactacttgggtatgtctctgttttccattttcaaaaataaatgaagcatgtaagtttttaattatttagttaagcctgttgttgtagtagttattagtttgtttatgcagttttcatgtttgggtttttgatacacaaaccctaattaagttcaattagtggatgtatctgcaagttagctgtctaagttaacctcttgttctttttttaatgtgatgcagtagtaccatgataccaaaaaaagaaaaaaaaaaa

SEQ ID NO: 173 - protein, 198 aa, Eschscholzia californica
MERTIVAFGAESSSSENTNMSTTNNSANSSAVATSSSSPPTLSRYESQKRRDWNTFGQYLKNHRPPLSLSRCSSAHVLEFLRYLDQFGKTKVHAQICPFFGHPNPPAPCPCPLRQAWGSLDALIGRLRAAFEENGGKPEANPFGARAIRLYLREVRDLQSKARGISYEKKKRKRPPNQLISTSSTQLPPSSSTNGATT

SEQ ID NO: 174 - DNA, 1255 bp, Eschscholzia californica
atgttcaaagaaaccccaagaagaagaagaagagataagaaacttcttaagtttcttcatcaattttatccttcaccaaacctcaaccataaaatccccctcctcctcttgggttataaagcatctgtctgaaagatcaaaattgtgagagttaagagtttagtagtagtccacaaaagaagcaaaatccaaacaactaggagccaaaagaagaacaaggagatatacttgatgtttccaagtaaattacatgttctcattcttcttccttgaagagaaacaaaccaaaggggagagcttgatcattctcatattcctcatcaacaacaaagtgaaattacactttttagatcatccaaatcttatatataatgagcatggaaacatcaatacttgcttttggaacagaaagctctagctcagagaacaccaacatgaactcaacaatggcagcaatatcatcatcttctccaccaaccctcagccgatacgaaagccaaaaacgtcgagactggaacacctttggccaatacctaaagaaccatcgacctccactttctctctcgtgctgtagtagtgctcatgttcttgaattccttcgctacctcgatcagtttggcaaaaccaaagttcacaaccacatctgtcccttctttggccacccaaacccacccggtccgtgcccttgtcctctccgtcaagcttggggaagcctcgatgctctcattggacgtctccgagccgcctttgaagagaacggcggaatgccggagaccaatccatttggagctcgagctattagactttacttgcgtgaagttcgcgattcacaatcgaaagcaagagggattagctatgagaagaagaaacgaaagcgtcttcctaatcaactaccttcatcaacacaagcattgctaccctcatcaagtaattcatgtaaatgagagaaattaactttctccattaccatgattatcagctcgtcaattactaattaaagcacctaaaggagattaattggttgaactattaatcaattacccaaactaaattaattagtaatttagtcttctaaatgggtagatctctattttcactttattcaataaatgaagcacatatgtatgcatgttgctaattagttaagtacttaagtttgtagcaactgtttttactgtaagttagtctgtatgtagttctttggttgtaaaaaccctaatttagtttatgtttaagtcaaaaaaaaaaaaaaaaa

SEQ ID NO: 175 - protein, 190 aa, Eschscholzia californica
MSMETSILAFGTESSSSENTNMNSTMAAISSSSPPTLSRYESQKRRDWNTFGQYLKNHRPPLSLSCCSSAHVLEFLRYLDQFGKTKVHNHICPFFGHPNPPGPCPCPLRQAWGSLDALIGRLRAAFEENGGMPETNPFGARAIRLYLREVRDSQSKARGISYEKKKRKRLPNQLPSSTQALLPSSSNSCK

SEQ ID NO: 176 - DNA, 1061 bp, Linum perenne
ctctctctaatcacttctcttcctccttcaaacttaatcaccatgtcaagcaacgaaagaggcaaggaactaatgggtgaaggatcaagcaccgaccctcactaccaccagcaacatcaccagcaccagcaccagcaaccgcctcagactccgagccggtacgagtcccagaagaggagggactggaacacgttcgggcagtacttaaagaaccagaggccaccggtcccactctcccactgcaactgcaaccacgtcatcgacttcctccgatacctcgaccagttcgggaagactaaggtacatatccagggttgcatgttttatgggcagcctgagccaccggctccctgcacctgccctctcaggcaggcgtgggggagcctggatgccctcataggcagactcagagctgcctatgaggagaacggtgggtctcctgagactaacccttttgccagcggggcgatcagggtttatcttagggaggttagggattgtcaggctaaggctagagggattccttacaagaagaagaagaagaaggcgacgggttcgggtggtggtggtgggagaggtggggatcatgatgatcagtctagctctgctcctccaatgcactactcttgatctggggattcggtggccatccgtatatatatgatgggactagtttcttttctgagtacacaacatgcatgagaagagactgtcttcctcttcttcaaggtcacgatcgaggaagctctatacgatcgatgaacgaaaggccaggcatgacgtattgctttaaataaaaaaaagagagaagatatggtttatggtatgcctagctagctagctatagcaatgcaagtgtttgtagctgggtctgatgaatttatatgcatgagatgagaaagtcctcttttataaactatctttcttctttggtttatgggttgctagctatatatatttgggggaagaatctgtaactcaaatcaatatacagatatgtgagtagctcgattgatgatgcatgctctaactttatctgaaattaagtttaatttatgtct

SEQ ID NO: 177 - protein, 195 aa, Linum perenne
MSSNERGKELMGEGSSTDPHYHQQHHQHQHQQPPQTPSRYESQKRRDWNTFGQYLKNQRPPVPLSHCNCNHVIDFLRYLDQFGKTKVHIQGCMFYGQPEPPAPCTCPLRQAWGSLDALIGRLRAAYEENGGSPETNPFASGAIRVYLREVRDCQAKARGIPYKKKKKKATGSGGGGGRGGDHDDQSSSAPPMHYS

SEQ ID NO: 178 - DNA, 1056 bp, Linum perenne
atctcttccccaaaattcttacactaatcatctctctttctttcattccgcctcaccgtcatgtcatcggcgcaagatcccacggcggcgccggaaggattctcctcctcccgaaccaaccaccaccaccaccaccccccgccgcaggcgccgctgagccgttacgagtcgcagaagcggcgggactggaacacgttcggacagtacttgaagaaccagaggccgccggtttccgtgtcccactgtggctcgacccacgtgctcgacttcctccggtacttggaccagttcgggaagacgaaggtccaccttaacggctgcgcgttcttcgggcagccggacccacctgccccctgcacctgcccgctgaggcaggcgtggggcagcctcgacgcgctcatcggtaggctgcgtgcagcctacgaggagcacggcgggccgcccgagacgaacccgtttgggaacggggcgatccgggtttacctgagggaagttagggagtgtcgggctaaggctagagggataccgtacaagaagaagaagaagaagaaggttaatcatcatcaggtggttaataaggtggcggccgccgttgttagggatcataataaggcggcggtggcggcggcggcgagcatgccgcagtgatcgggtctgtgttcgtgagagcaagctggttcagcaaatatcagatgaatcatggttgaagaaggaaattaatctgcatgcatgggagagctcatggctgatcaatgaccatcaagactttgtataatatgtataatcaatcaatcaatgaatcacttttataatctcagaaagtagttccaaggaatccttctgctgctgctgctgctgctgctttctgccattctatattattatatagatattcatagagatatagatatataatgaccggtacgcatctcaagttgttttgatagaagaatatgatctgtaatggctaaatttctgggttcctagcttcaattaatgtcgcaaagaacgttaacttttgcgcggtcaaattattgagatataatggttcatgac

SEQ ID NO: 179 - protein, 195 aa, Linum perenne
MSSAQDPTAAPEGFSSSRTNHHHHHPPPQAPLSRYESQKRRDWNTFGQYLKNQRPPVSVSHCGSTHVLDFLRYLDQFGKTKVHLNGCAFFGQPDPPAPCTCPLRQAWGSLDALIGRLRAAYEEHGGPPETNPFGNGAIRVYLREVRECRAKARGIPYKKKKKKKVNHHQVVNKVAAAVVRDHNKAAVAAAASMPQ